(19) United States
(12) Patent Application Publication
(10) Pub. No.: US 2024/0335588 A1
(43) Pub. Date: Oct. 10, 2024

(54) Optimized Biomaterials of Various Cells and Tissues from Sustainable Sources

(71) Applicant: New York University in Abu Dhabi Corporation, Abu Dhabi (AE)

(72) Inventors: Mano Govindharaj, Abu Dhabi (AE); Noura Sayed AlHashimi, Abu Dhabi (AE); Soja Saghar Soman, Abu Dhabi (AE); Vijayavenkataraman Sanjairaj, Abu Dhabi (AE)

(21) Appl. No.: 18/749,241

(22) Filed: Jun. 20, 2024

Related U.S. Application Data
(63) Continuation of application No. PCT/IB22/00783, filed on Dec. 23, 2022.
(60) Provisional application No. 63/293,393, filed on Dec. 23, 2021.

(51) Int. Cl. A61L 27/34; A61L 27/26; A61L 27/52; A61L 27/54; C12N 5/074; C12N 5/077; C12N 5/0775; C12N 5/0797

(52) U.S. Cl. CPC A61L 27/34; A61L 27/26; A61L 27/52; A61L 27/54; C12N 5/0623; C12N 5/0656; C12N 5/0662; C12N 5/0696; A61L 2300/62; A61L 2420/08; C12N 2513/00; C12N 2533/54; C12N 2533/74; C12N 2533/78; C12N 2533/90

(57) ABSTRACT

The present disclosure relates to biomaterials derived from sustainable sources. Described herein are biomaterials and tissue engineering constructs comprising components derived from sustainable sources such as from tunicates, fish skin and bananas.

### CROSS-REFERENCE TO RELATED APPLICATIONS

This application claims priority to U.S. Provisional Application No. 63/293,393, filed on Dec. 23, 2021, which is incorporated herein by reference in its entirety.

### BACKGROUND OF THE INVENTION

Bioprinting is an emerging technology with various applications in making functional tissue constructs to replace injured or diseased tissues. It is a relatively new approach that provides high reproducibility and precise control over the fabricated constructs in an automated manner, potentially enabling high-throughput production.

During the bioprinting process, a solution of one or more biocomponentsor a mixture of several biocomponents in the hydrogel form, usually encapsulating the desired cell types, termed the bioink, is used for creating tissue constructs. This bioink can be cross-linked or stabilized during or immediately after bioprinting to generate the final shape, structure, and architecture of the designed construct. Bioinks may be made from natural or synthetic biocomponents alone, or a combination of the two as hybrid materials. In certain cases, cell aggregates without any additional biocomponents can also be adopted for use as a bioink for bioprinting processes.

Thus, there is a need in the art for novel biomaterials derived from sustainable sources for tissue engineering applications, such as forming bioinks.

### SUMMARY OF THE INVENTION

In some aspects, the present invention relates to a biomaterial comprising at least one component derived from at least one sustainable source. In some embodiments, the at least one sustainable source is selected from the group consisting of: tunicate, fish skin, algae, banana skin or stem, and watermelon. In some embodiments, the at least one component is selected from the group consisting of: extracellular matrix (ECM), ECM proteins, decellularized extracellular matrix (dECM), lyophilized dECM, collagen, cellulose, cellulose microfibers (CMFs) and alginate.

In some embodiments, the biomaterial further comprises at least one additive. In some embodiments, the at least one additive is selected from the group consisting of: biopolymers, synthetic polymers, cross-linking agents, surfactants, and drugs/therapeutics. In some embodiments, the biomaterial is selected from the group consisting of: bioink, hydrogels, microparticles, wound dressing, tissue engineering constructs, scaffolds, substrates and tunneling wound fillers (TWFs).

In some embodiments, the biomaterial further comprises one or more cells. In some embodiments, the one or more cells is selected from the group consisting of: fibroblasts, neural stem cells, or mesenchymal stem cells. In some embodiments, the one or more cells are derived from induced pluripotent stem cells.

In some embodiments, the at least one sustainable source is a tunicate. In some embodiments, the at least one component comprises dECM. In some embodiments, the tunicate is the species Polyclinum constellatum or species Pallusia nigra.

In some embodiments, the biomaterial further comprises mesenchymal cells. In some embodiments, the biomaterial comprises a bioink. In some embodiments, the biomaterial comprises a hydrogel. In some embodiments, the biomaterial further comprises alginate. In some embodiments, the biomaterial further comprises Matrigel. In some embodiments, the biomaterial comprises (a) collagen from fish skin, and (b) a CMFs from banana. In some embodiments, the collagen is derived from grouper. In some embodiments, the CMFs are derived from banana stem. In some embodiments, the biomaterial comprises a bioink. In some embodiments, the biomaterial comprises alginate. In some embodiments, the biomaterials further comprises Matrigel.

In some embodiments, the biomaterial is a tunneling wound filler (TWF). In some embodiments, the TWF comprises a tri-layer coating comprising a first layer comprising CMF, a second layer comprising collagen, and a third layer comprising collagen. In some embodiments, the biomaterial comprises mesenchymal stem cells. In some embodiments, the biomaterial further comprises Baneocin.

In some aspects, the present invention relates to a method having the steps of generating tunicate-derived decellularized extracellular matrix (dECM) by decellularizing tunicate tissue, lyophilizing the decellularized tissue, and forming a scaffold from the tunicate-derived dECM. In some embodiments, the method further comprises powderizing the dECM and forming a bioink from the powderized dECM. In some embodiments, the method comprises 3D printing the bioink.

In some aspects, the present invention relates to a kit comprising a 3D printer and a bioink comprising tunicate-derived decellularized extracellular matrix (dECM).

In some aspects, the present invention relates to a method for generating a sustainable biomaterial, having the steps of isolating CMFs from banana, harvesting collagen from fish skin, coating the banana-derived CMFs with the fish-derived collagen, coating the collagen coated CMFs with an additional layer of fish-derived collagen, and combining with Sodium Alginate. In some embodiments, the method further comprises powderizing the biomaterial and forming a bioink from the powderized biomaterial. In some embodiments, the method comprises 3D printing the bioink.

In some aspects, the present invention relates to a kit comprising a 3D printer and a bioink comprising fish-derived collagen and banana-derived Cellulose Microfibers (CMFs).

In some aspects, the present invention relates to a kit comprising a 3D printer and a bioink for printing tunneling wound fillers (TWFs) comprising fish-derived collagen and banana-derived Cellulose Microfibers (CMFs).

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A and FIG. 1B shows colonial tunicate species Polyclinum constellatum growing on the ship hull and port structures. FIG. 1C and FIG. 1D shows a small colony of tunicates removed from its habitat, showing various color morphs such as brown, red, green, and honey-colored. FIG. 1E shows the external morphology of a single tunicate. FIG. 1F shows a cross-sectional view of the tunicate showing jelly-like consistency. FIG. 1G and FIG. 1H shows a cross sectional view of the tunicates after removal of the top tunic layers, showing the cellulose fibrillar networks. FIG. 1I and FIG. 1J shows the flexibility and easy handleability of the tunic.

FIG. 2A shows the external morphology of as-harvested tunicate showing a spiny-eyed structure of a Pineapple skin. FIG. 2B shows magnified image of the spiny-eye revealing the pores through which water intake and exudation happens.

FIG. 2C shows the presence of residues of phytoplanktons and zooplanktons near the pores on the tunicate surface. FIG. 2D, FIG. 2E, FIG. 2F, FIG. 2G, FIG. 2H, and FIG. 2I show the morphology of the (decellularized) lyophilized tunic, showing the presence of cellulose crystals and nanofibers (indicated by red arrows). FIG. 2J and FIG. 2K show the AFM topological images of the lyophilized tunicate surface.

FIG. 3A shows the FT-IR spectra of as-harvested, decellularized, and lyophilized tunic of Polyclinum constellatum. FIG. 3B shows the Raman spectra of the (decellularized) lyophilized tunic. FIG. 3C shows the X-ray diffractogram of the (decellularized) lyophilized tunic, showing sharp peaks, indicating the presence of cellulose crystals. FIG. 3D shows the TGA thermogram of the (as-harvested) lyophilized and (decellularized) lyophilized tunic. FIG. 3E shows the stress-strain curve (tensile) of as-harvested, decellularized, and lyophilized tunic of Polyclinum constellatum.

FIG. 4A shows the external appearance of as-harvested, decellularized, lyophilized and rewetted (with PBS) tunic of Polyclinum constellatum (*** represents p≤0.0005). FIG. 4B shows the swelling behavior of the lyophilized tunic. FIG. 4C, FIG. 4D, FIG. 4E and FIG. 4F shows a circle of 1 cm diameter from changes in the weight and dimensions of as-harvested, decellularized, lyophilized and rewetted tunics in different external morphologies. FIG. 4G, FIG. 4H and FIG. 4I, and FIG. 4J shows a rectangle of dimensions 0.5×2 cm. FIG. 4K, FIG. 4L, FIG. 4M, and FIG. 4N shows a square of sides 1 cm.

FIG. 5A shows metabolic activity of MEFs seeded on lyophilized tunics assessed using Alamar Blue assay. FIG. 5B shows DNA quantification of MEFs seeded on lyophilized tunics assessed using Pico green-dsDNA quantification assay. FIG. 5C shows the protein quantification of MEFs seeded on lyophilized tunics assessed using BCA protein quantification assay (n=9, *, #, and \hat{ } indicate significant differences compared to day 1, 3, and 5 respectively and *, #, \hat{ } represents p≤0.05, **, ##, \hat{ }\hat{ } represents p≤0.005, and ***, ## #, \hat{ }\hat{ }\hat{ } represents p≤0.0005). FIG. 5D, FIG. 5E, and FIG. 5F show SEM image of MEFs seeded on Day 1, 5 and 7 respectively. FIG. 5G, FIG. 5H, and FIG. 51 show live/dead image of MEFs seeded on Day 1, 2 and 3 respectively.

FIG. 6A and FIG. 6B depict exemplary bioprinted tissue constructs-MEFs suspended in tunic dECM-alginate bioink. FIG. 6C shows the metabolic activity of MEFs post-bioprinting assessed using Alamar Blue assay. The metabolic activity was statistically significant on day 7 when compared to day 1, 3 and 5. (n=3, ns non-significant *, #, and \hat{ } indicate significant differences compared to day 1, 3, and 5 respectively and *, #, \hat{ } represents p<0.05, *, #, \hat{ }\hat{ } represents p≤0.005, and ***, ###, \hat{ }\hat{ }\hat{ } represents p≤0.0005). FIG. 6D, FIG. 6E, and FIG. 6F show a light microscopic image, Live/Dead staining and DAPI staining of bioprinted tissue constructs on Day 1 respectively. (Scale: 100 μm). FIG. 6G, FIG. 6H, and FIG. 6I show a light microscopic image, Live/Dead staining and DAPI staining of bioprinted tissue constructs on Day 7 respectively (Scale: 100 μm).

FIG. 7A depicts an exemplary wound-dressing materials comprising the biomaterial of the disclosed invention, two commercial wound-dressing materials (Euromed® and Gazin®), and a cotton cloth. FIG. 7B shows a macerating artificial wound model for studying the exudate absorption of various wound-dressing materials. FIG. 7C shows results for exudate absorption studies of Cotton cloth, Euromed® and Gazin®, and the lyophilized tunic of the present invention. Green line represents the breaking point beyond which there was fluid leakage out of the dressing material. FIG. 7D shows the fluid absorption capacity of various wound dressing materials, measured at the breaking point (mL/g) using camel blood plasma. (n=3, *, #, and \hat{ } indicate significant differences compared to cotton cloth, Gazin®, and Euromed® respectively and *, #, \hat{ } represents p≤0.05, **, ##, \hat{ }\hat{ } represents p<0.005, and **, ###, \hat{ }\hat{ }\hat{ } represents p≤0.0005). FIG. 7E shows the wound exudate absorption capacity of the lyophilized tunic of mass 0.0197 g after a 48-hr incubation in camel blood plasma. (n=3, *** represents p≤0.0001). FIG. 7F shows the external appearance of the tunic as a dressing material before incubation and after 48-hr incubation in camel blood plasma.

FIG. 7G shows the flexibility of the tunic after exudate absorption aiding in ease of removal from the wound site.

FIG. 8 shows the changes in the weight and dimensions of as-harvested, decellularized, lyophilized and rewetted tunics in different external morphologies of circles of 2 and 3.5 cm diameter

FIG. 9 shows the changes in the weight and dimensions of as-harvested, decellularized, lyophilized and rewetted tunics in different external morphologies of rectangles of dimensions 1×2 cm and 1.5×2 cm.

FIG. 10 shows the changes in the weight and dimensions of as-harvested, decellularized, lyophilized and rewetted tunics in different external morphologies of squares of sides 1.5 and 2 cm.

FIG. 11 is a schematic illustrating an exemplary method of using tunicates to produce dECM scaffolds and bioink for 3D bioprinted scaffolds according to aspects of the present invention.

FIG. 12A and FIG. 12B shows an exemplary method for the preparation of tunicate dECM scaffolds and bioinks for differentiating NSCs to peripheral nerve tissues according to aspects of the present invention. Fresh tunicates were harvested from the coastal regions of UAE and processed in the lab. FIG. 12A shows the preparation of sterile dECM scaffolds, seeding of NSCs, in vitro culture, induction of PN differentiation and formation of PN on the dECM scaffolds. FIG. 12B shows the dECM was processed into a sterile powder and the bioink was formulated and optimized by incorporating the NSCs. The neural tissue constructs were printed and cultured in vitro. The differentiated PN was characterized after the PN induction. The results proved bioprinting as a promising method for translational medicine applications that support tissue viability, tissue differentiation and tissue storage.

FIG. 13A through FIG. 13N shows the NSC to PN differentiation in dECM tunicate scaffolds. FIG. 13A, FIG. 13B, and FIG. 13C show the NSC-loaded dECM tunicate scaffolds under the light microscope: FIG. 13A shows a dECM tunicate scaffold without cells. FIG. 13B shows a scaffold just after cell loading, the cells appear rounded and floated over the scaffold. FIG. 13C shows cells attached to the scaffolds on day 3 of seeding just before PN induction. The scaffolds appeared thick and impermeable to light by day 3 of seeding (scale bars=125 μm). FIG. 13D, FIG. 13E, and FIG. 13F are SEM images of the NSC-loaded tunicate dECM scaffolds: FIG. 13D shows day 3 of NSC culture on the scaffolds showing fibroblast-like morphology.

FIG. 13E shows the NSCs changed to more rounded cell appearance by day 7. FIG. 13F shows formation of extended peripheral neuron fibers by day 12 of PN induction (scale bars=50 μm). FIG. 13G shows the no-cell scaffold control with DAPI and NEFH staining. FIG. 13H shows DAPI and NEFH staining on day 3 of PN induction. FIG. 13I shows the expression of NEFH protein on day 12 in the induced NSCs-loaded scaffolds indicate the differentiation of the NSCs to PN (scale bars=100 μm). FIG. 13J, FIG. 13K, and FIG. 13L show live-dead staining on cells grown on the tunicate dECM scaffolds showed an increase in number of green florescent cells over time and proliferation of thread-like structures by day 12 (scale bars=100 μm). FIG. 13M shows gene expression studies using qPCR experiments; the relative mRNA expression of NEFH and PRPH expression was increased to four-to five-fold on day 12 of the PN induction, compared to the day 3 samples, while the stemness marker HNKI expression was remarkably reduced, indicating the differentiation of NSCs to PN. Day 12 neurons also showed increased levels of the pan neuronal marker TUBB3. The gene expression was normalized to house-keeping gene GAPDH. Data reported as mean+/−SD (n=3; *p<0.05, ** p<0.01, *** p<0.001, by two-tailed students t test between test and control samples. FIG. 13N show the Alamar blue cell proliferation assay showed significant cell proliferation on day 12 compared to day 3, p<0.05.

FIG. 14A through FIG. 14M shows a standardization of exemplary bioink, crosslinking and bioprinting of neural tissue constructs according to aspects of the present invention. FIG. 14A shows 10% tunicate hydrogel in NSC media extruded into PBS, the filament is not dense enough to free flow into the solution, as shown by its upward push when it extrudes into the solution. The filaments broke and fell into the solution when extruded continuously. FIG. 14B shows 10% tunicate gel+26% Matrigel in NSC media in PBS, the filament is smoothly flowing into the solution. The filaments broke and fell into the solution when extruded continuously for a longer time.

FIG. 14C shows 10% tunicate gel in NSC media extruded into the crosslinking solution (250 mM CaCl2) gets crosslinked but lack smooth flow. FIG. 14D shows 10% tunicate gel+26% Matrigel in NSC media extruded into the crosslinking solution (250 mM CaCl2) shows a seamless extrusion. FIG. 14E shows filament formation of 10% tunicate hydrogel in NSC media without Matrigel. FIG. 14F shows filament formation of 10% tunicate gel+26% Matrigel in NSC media. FIG. 14G shows droplet formation of 10% tunicate hydrogel in NSC media without Matrigel at the tip of the needle. FIG. 14H shows droplet formation of 10% tunicate gel+26% Matrigel in NSC media at the tip of the needle. FIG. 14I depicts an exemplary BioCAD design of the tissue construct. FIG. 14J shows a tool path generated using the BioCAD software showing the direction of printhead movement. The numbers represent the steps in printhead movement. FIG. 14K shows a bioprinted neural tissue construct. FIG. 14L shows a lattice coordinate profile showing structural uniformity. The upward wave shows the mean struct length and the downward wave shows the total strut thickness. FIG. 14M shows a bioprinted tissue constructs in a 24 well plate printed using the well editor software plugin. The dimensions of the bioprinted tissue constructs were 8 mm×8 mm×1 mm. Alcian blue dye was used to enhance the visibility of cell-free hydrogel filaments. Neural tissue constructs were printed without the dye.

FIG. 15A through FIG. 15K shows the NSC to PN differentiation of the bioprinted tissues. FIG. 15A, FIG. 15B, and FIG. 15C show live-dead staining on bioprinted NSCs in optimized bioink containing the dECM powder, with 26% Matrigel. FIG. 15D, FIG. 15E, and FIG. 15F show live-dead staining on bioprinted NSCs in optimized bioink containing the dECM powder, without Matrigel. FIG. 15A, FIG. 15B and FIG. 15C show an increase in the number of green florescent cells over time and proliferation of thread-like structures by day 12. FIG. 15D, FIG. 15E, and FIG. 15F show a Tunicate dECM bioink without Matrigel did not favor cell proliferation, evident from the lesser number of green fluorescing cells throughout the culture. FIG. 15G, FIG. 15H, and FIG. 15I shows live-dead staining of cell-free (control) hydrogel constructs. Scale bars=100 μm. FIG. 15J shows a part of the whole tissue construct, one-hour post-bioprinting, showing viable green fluorescing cells in the entire tissue construct. FIG. 15K shows Alamar blue cell proliferation assay showed significant cell proliferation on day 12 (p<0.0001) and day 7 (p<0.001) compared to day 1 in optimized bioink containing the dECM powder and Matrigel.

FIG. 16A through FIG. 16M show NSC to PN differentiation in bioprinted neural tissue constructs. FIG. 16A shows DAPI staining on day 1 post-printing. FIG. 16B shows DAPI staining and NEFH immunofluorescence staining on day 3 of the neural induction showed no NEFH expression. FIG. 16C shows DAPI staining and NEFH immunofluorescence staining on day 12 of the neural induction.

FIG. 16D, FIG. 16E, FIG. 16F showed high NEFH expression, indicating PN differentiation. Scale bars=100 μm. FIG. 16G, FIG. 16H, and FIG. 16I show SEM images of the bioprinted tissue constructs on day 3. FIG. 16J, FIG. 16K, and FIG. 16L show SEM images of the bioprinted tissue constructs on day 7. SEM images of the differentiated neural tissue constructs on day 12 showed remarkable neural cell morphology and neural filament formation (yellow arrows), compared to day 3 and day 7 post-induction. FIG. 16M shows the direction of neural filament formed were perpendicular to the printing direction (blue arrows), which requires further investigation. mRNA expression of PN markers; PRPH and NEFH were upregulated on day 12 of PN induction compared to day 3. The stemness marker HNKI was significantly downregulated and the change in the pan neural marker TUBB3 was non-significant (NS). Data reported as mean+/−SD (n=3; * p<0.05, *** p<0.001, by two-tailed students t test between test and control samples).

FIG. 17A through FIG. 17D show the freeze-thaw study of the dECM-grown and bioprinted neurons. FIG. 17A shows the cell proliferation of PN on dECM scaffold evaluated using Alamar Blue assay showed less growth on day 3, then recovered proliferation on day 7, but still showed less cells compared to day 1. FIG. 17B shows the live-dead staining of day 7 dECM scaffold with PN; very less live cells noticed compared to bioprinted tissues. FIG. 17C shows the cell proliferation of PN on bioprinted tissues evaluated using Alamar Blue assay also showed less cells on day 3 compared to day 1. FIG. 17D shows the cells recovered faster and showed two-fold growth by day 7 compared to day 1. Live-dead staining also showed more live cells in the bioprinted tissue constructs. For Alamar Blue assay results (FIG. 17A and FIG. 17C), viable cells recovered from the frozen tissues on day 1 was considered as 100% and relative cell proliferation on day 3 and day 7 were calculated in Alamar blue assay (*** p<0.001, **** p<0.0001). Scale bars=500 μm.

FIG. 18 is a schematic illustrating an exemplary method of using tunicates to produce dECM scaffolds and bioink for 3D bioprinted scaffolds according to aspects of the present invention.

FIG. 19A shows a morphological analysis of solitary marine tunicate (P. nigra) attached and growing on marine structures. FIG. 19B shows Tunicate growing in the marine environment showing separate water entrance and exit tubes (siphons). FIG. 19C shows a velvety black and dark brown colored sac-shaped body. FIG. 19D and FIG. 19E show SEM micrographs of external morphology of as-harvested tunicate showing honeycomb-shaped structure.

FIG. 20A shows that decellularized tunicates provides translucent materials. FIG. 20B and FIG. 20C shows the morphology of the P. nigra thick translucent leathery and flexible materials. FIG. 20D and FIG. 20E show SEM micrographs of tunic materials preserve the naturally predesigned honeycomb-shaped structure after decellularization (indicated by red arrows). FIG. 20F shows the DNA content of the as-harvested and the dECM materials.

FIG. 21A through FIG. 21H depicts an exemplary method for the preparation of a decellularized ECM hydrogel from tunicate (P. nigra) according to aspects of the present invention. FIG. 21A shows the tunicate after sample collection. FIG. 21B shows the decellularized tunicate. FIG. 21C shows the freeze-dried tunicate pieces. FIG. 21D shows the cryogenic homogenized dECM powder. FIG. 21E shows the Pepsin/HCl digested create a solubilized dECM tunicate matrix which is liquid at 4° C. FIG. 21F shows Lyophilized dECM powder. FIG. 21G depicts an exemplary dECM bio-ink preparation. FIG. 21H depicts an exemplary 3D printed dECM bio-ink.

FIG. 22A shows the FT-IR spectra of as-harvested, decellularized, and lyophilized tunic of P. nigra. FIG. 22B shows the X-ray diffractogram of the (decellularized) lyophilized tunic, showing sharp peaks, indicating the presence of cellulose crystals. FIG. 22C shows the TGA thermogram of the (as-harvested) lyophilized and (decellularized) lyophilized tunic. FIG. 22D shows the Raman spectra of the (decellularized) lyophilized tunic. FIG. 22E shows the swelling behavior of the lyophilized tunic material P. nigra (*** represents p≤0.0005). FIG. 22F shows the stress-strain curve (tensile) of as-harvested, decellularized, and lyophilized tunic of P. nigra.

FIG. 23A through FIG. 23G show the viability of hMSCs cultured within tunic dECM scaffold. FIG. 23A shows day 1, FIG. 23B shows day 5, and FIG. 23C shows day 7 in culture, respectively (green: viable cells, red: dead cells). Demonstrating the viability of cell in all time point of infestation indicating absence of cytotoxic effect of dECM scaffold obtained after decellularization. FIG. 23D and FIG. 23E, and FIG. 23F show representative SEM images indicate the hMSCs morphology and attachment (FIG. 23D), spread (FIG. 23E) and align along the dECM scaffold. FIG. 23G shows metabolic activity of hMSCs in dECM scaffolds were assessed by Alamar Blue assay. Metabolic activity of hMSCs on dECM scaffolds was statistically significant on day 7 when compared to day 1. (n=3, ns non-significant, * represents p≤0.05, ** represents p≤0.005, and *** represents p≤0.0005

FIG. 24A through FIG. 24M show results of staining of 3D printed bioprinted tissue constructs. FIG. 24A, FIG. 24B and FIG. 24C show Live/Dead staining of 3D bioprinted tissue constructs on Day 1, 5 and 7 (Scale: 100 μm). FIG. 24D, FIG. 24E, and FIG. 24F show SEM image of hMSCs on 3D bio printed hydrogel scaffold at Day 1. FIG. 24G, FIG. 24H, and FIG. 24I show SEM image of hMSCs seeded on Day 5. FIG. 24K and FIG. 24L show SEM image of hMSCs on dECM scaffold at day 7. FIG. 24M shows metabolic activity of hMSCs in both dECM scaffold and post-bioprinting scaffolds were assessed by Alamar Blue assay. The metabolic activity was statistically significant on day 7 compared to day 3. (n=3, ns non-significant, * represents p≤0.05, ** represents p≤0.005, and *** represents p≤0.0005.

FIG. 25A through FIG. 25D shows a histological analysis of chondrogenic differentiation in vitro. FIG. 25A and FIG. 25B shows a visualization of glycosaminoglycans deposition through alcian blue staining of hMSCs on 3D bioprinted hydrogel construct and dECM scaffold and day 14 and 21 (Scale: 100 μm). FIG. 25C and FIG. 25D show the detection of GAG deposition through safranin O staining on 3D bio printed hydrogel construct and dECM scaffold during chondrogenesis on Day 14 and 21 (Scale: 100 μm).

FIG. 26A through FIG. 26D shows the PCR analysis of collagen I and collagen IIa gene expression in chondrogenically stimulated and non-stimulated control samples. Collagen I and Collagen IIa gene expression in bioprinted hMSCs indicating chondrocyte differentiation. FIG. 26A shows the Collagen I showed significant expression on days 14 and 21 compared to day 1. FIG. 26B shows the Collagen IIa started to express on day 14 and showed ˜7-fold expression by day 21. FIG. 26C shows the gene expression of hMSCs cultured on the dECM scaffold showed significant expression on days 14 and 21 compared to day 1. FIG. 26D shows the Collagen IIa peaked expression on day 14, in contrast, on day 21 the expression was declined. The gene expression of the housekeeping gene GAPDH was determined to verify the usage of equal amounts of RNA for RT-PCR. The data reported as mean+/−SD (n=3 in triplicate; *** p<0.001, **** p<0.00001 by two-tailed students t-test between test and control samples. (FIG. 26A and FIG. 26B).

FIG. 27 shows a scheme describing an exemplary method, including chemical and mechanical procedures used to isolate Cellulose Microfibers (CMFs) from banana stem, according to aspects of the present invention.

FIG. 28 shows a scheme describing an exemplary method of preparing a coated biomaterial according to aspects of the present invention. Shown is a primary coating on CMFs and secondary coating after 3D printing of collagen coated CMF.

FIG. 29A and FIG. 29B shows a scheme describing an exemplary method for fabrication of 3D printed tunnelling wound fillers (TWFs) (FIG. 29A), and a 3D bioprinting process of 50 CMFs/COL-based TWFs with human Mesenchymal Stem Cells (hMSCs) (FIG. 29B).

FIG. 30A through FIG. 30F shows a formulation of CMFs/COL and CMFs/COL/ALG for fabrication of tunneling wound fillers (TWFs). FIG. 30A shows a process of collagen coating on CMFs. FIG. 30B and FIG. 30C shows the air drying of collagen-coated CMFs. FIG. 30D, FIG. 30E, and FIG. 30F show three different concentrations of CMFs/COL/ALG hydrogel.

FIG. 31A through FIG. 31F shows SEM micrographs of CMFs. FIG. 31A, FIG. 31B and FIG. 31C show CMFs without primary collagen coating. FIG. 31D shows smooth surface of non-collagen-coated cellulose fibers. FIG. 31E, FIG. 31F shows CMFS with primary collagen coating and the rough surface of the collagen-coated cellulose fibers.

FIG. 32A shows FT-IR spectra of CMFs, Collagen (COL), and CMFs/COL hydrogel. FIG. 32B shows Raman spectra of CMFs, Collagen (COL), and CMFs/COL hydrogel. FIG. 32C shows a TGA thermogram of the CMFs, Collagen (COL), and CMFs/COL hydrogel.

FIG. 33A through FIG. 33G shows results for 3D printing of exemplary biomaterials according to aspects of the present invention. FIG. 33A is the 3D printability of 25 CMF/COL/ALG. FIG. 33B is the 3D printability of 50 CMF/COL/ALG. FIG. 33C is the 3D printability of 5 CMF/COL/ALG hydrogel. FIG. 33D shows the rheological properties of the exemplary biomaterials. FIG. 33E shows the swelling ratio of the exemplary biomaterials. FIG. 33F shows the degradation rate of the exemplary biomaterials. FIG. 33G shows 3D printed Tunneling wound filler (TWF) with 50 CMFs/COL/ALG bioink.

FIG. 34A through FIG. 34F shows SEM micrographs of secondary collagen coating and non-coating on cellulose-based 3D printed TWFs. FIG. 34A, FIG. 34B, and FIG. 34C show smooth surfaces of non-collagen-coated TWFs. FIG. 34D, FIG. 34E and FIG. 34F show rough surfaces of the TWFs indicates the post-printing collagen coating.

FIG. 35 shows a collagen release profile, and Drug (Baneocin) release profile from an exemplary biomaterial of the present invention.

FIG. 36A through FIG. 36K is Live/Dead staining of hMSCs in 3D bioprinted TWF tissue constructs on days 3, 5, and 7 (green: viable cells). FIG. 36A, FIG. 36D, and FIG. 36G show control groups without cells. FIG. 36B, FIG. 36E, and FIG. 36H show fluorescent staining of cells counterstained by Calcein AM and Ethidium homodimer1. FIG. 36C, FIG. 36F, and FIG. 36I show fluorescent staining of cell nuclei in bioprinted TWF tissue constructs counterstained by DAPI. FIG. 36J shows 3D bioprinted TWF constructs. FIG. 36K shows Alamar blue assay bar graph on days 1, 3, and 7 for cell proliferation. Data reported as mean±SD (n=3, *p≤0.05).

FIG. 37A through FIG. 37L shows a scratch test to evaluate in vitro wound healing. FIG. 37A, FIG. 37E, and FIG. 371 show the control group. FIG. 37B, FIG. 37F, and FIG. 37J show the group treated with alginate extract. FIG. 37C, FIG. 37G, and FIG. 37K show the group treated with ALG./COL extract. FIG. 37D, FIG. 37H, and FIG. 37L show the group treated with ALG/CMF/COL extract showing fastest migration of fibroblasts.

FIG. 38A though FIG. 38D shows a chicken tissue model to test the suitability of TWFs in tunnelling wound management. FIG. 38A and FIG. 38B show 3D Printed 50 CMFs/COL/ALG-based TWFs. FIG. 38C shows a wound model of diameter 5 mm and length 1.5 cm (before packing with TWF). FIG. 38D shows packing of the tunnel wounds with 3D printed TWFs (after packing with TWF).

### DETAILED DESCRIPTION

Sustainable sources for components used to formulate biomaterials may be found throughout civilization and nature. For example, processing of vegetables and fruits for human consumption provides a large source of cellulose and cellulose fibers in the discarded stems, skins or peels. Many sustainable sources are provided by nature, particularly in collagen from discarded fish skins, and extracellular matrix harvested from invasive species like tunicates. The use of these biomaterials derived from sustainable sources can reduce the cost associated with producing mammalian-derived bioinks, as they are more readily available and cheaper to produce.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, ±10%, ±5%, ±1%, or ±0.1% from the specified value, as such variations are appropriate to perform the disclosed methods.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

As used herein, “extracellular matrix composition” includes both soluble and non-soluble fractions or any portion thereof. The non-soluble fraction includes those secreted ECM proteins and biological components that are deposited on the support or scaffold. The soluble fraction includes refers to culture media in which cells have been cultured and into which the cells have secreted active agent(s) and includes those proteins and biological components not deposited on the scaffold. Both fractions may be collected, and optionally further processed, and used individually or in combination in a variety of applications as described herein.

As used herein, a “graft” refers to a cell, tissue, organ, or biomaterial that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft,” “allogeneic transplant,” “allogeneic implant,” and “allogeneic graft.” A graft from an individual to his identical twin is referred to herein as an “isograft,” a “syngeneic transplant,” a “syngeneic implant” or a “syngeneic graft.” A “xenograft,” “xenogeneic transplant,” or “xenogeneic implant” refers to a graft from one individual to another of a different species. The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, “polymer” includes copolymers. “Copolymers” are polymers formed of more than one polymer precursor. Polymers as used herein include those that are soluble in a solvent that are insoluble in an antisolvent.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, “tissue engineering” refers to the process of generating a tissue ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein, the terms “tissue grafting” and “tissue reconstructing” both refer to implanting a graft into an individual to treat or alleviate a tissue defect, such as a lung defect or a soft tissue defect.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, tissue derived adult stromal cell or other such progenitor cell, that is not fully differentiated when incubated in the medium, develops into a cell with some or all of the characteristics of a differentiated cell.

The term “derived from” is used herein to mean to originate from a specified source.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the term “multipotential” or “multipotentiality” is meant to refer to the capability of a stem cell to differentiate into more than one type of cell.

As used herein, a “pluripotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of 3H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to biomaterials with components derived from sustainable sources. In some examples, the biomaterials comprise bioinks to be used in bioprinting. In other examples, the biomaterials are hydrogels or microparticles for tissue engineering applications. In some embodiments, the biomaterials are Tunneling Wound Fillers (TWFs) for surgical implantation.

Aspects of the invention relate to biomaterials comprising components derived from at least one sustainable source. Example sustainable sources are provided herein, including, but not limited to, sources such as, marine-life, fruits, vegetables, plants, kitchen waste, and the like.

In some embodiments, the biomaterial comprises components derived from environmentally harmful marine organisms. In some embodiments, the biomaterial comprises components derived from marine organisms such as, but not limited to, barnacles, tunicates and fish skins. In some embodiments, the biomaterial comprises components derived from tunicates. In some embodiments, the biomaterial comprises components derived from algae. In some embodiments, the biomaterial comprises components from fish skin, such as discarded fish skin. In some embodiments, the biomaterial comprises components derived from marine organism shells. In some embodiments, the biomaterial comprises components derived from barnacles. In some embodiments, the biomaterial comprises components derived from mollusks.

In some embodiments, the biomaterial comprises components derived from plants, fruits, and vegetables, such as waste products of plants, fruits, and vegetables. In some embodiments, the biomaterial comprises components derived from banana stem or peels. For example, in some embodiments, the biomaterial comprises banana stem from a banana tree or plant. In some embodiments, the biomaterial comprises components derived from watermelon or watermelon rinds. Although some examples are provided, any sustainable sources for deriving components for biomaterials may be used as would be known by one of ordinary skill in the art.

In some embodiments, the components derived from the sustainable source may include, but are not limited to, extra-cellular matrix (ECM), ECM proteins, collagen, cellulose, cellulose microfibers (CMFs), and alginate. In some embodiments, the components derived from sustainable sources may include Chitosan, and other cross-linking agents such as photoinitiators like Igracure, Riboflavin, and Lithium phenyl-2,4,6-trimethylbenzoylphosphinate (LAP), for photocrosslinking. In some embodiments, the components derived from sustainable sources may include sodium alginate for calcium chloride ionic crosslinking or other chemical reagents for chemical crosslinking.

In some embodiments, the biomaterial comprises at least one component derived from a sustainable source and other additives, including, but not limited to biopolymers, synthetic polymers, cross-linking agents, surfactants, and drugs/therapeutics. In some embodiments, the biomaterial further comprises Matrigel. In some embodiments, the biomaterial comprises between 5-50% Matrigel. In some embodiments, the biomaterial comprises between 10-40% Matrigel. In some embodiments, the biomaterial comprises between 20-30% Matrigel. For example, in some embodiments, the biomaterial comprises 26% Matrigel.

Aspects of the invention relate to biomaterials comprising one or more components derived from tunicates. For example, in certain embodiments, the present invention provides biomaterials comprising tunicate-derived components such as extracellular matrix (ECM). In some embodiments, the biomaterial comprises decellularized tunicate-derived ECM. In some embodiments, the biomaterial comprises lyophilized tunicate-derived ECM. In some embodiments, the biomaterial comprising decellularized and lyophilized tunicate-derived ECM. In some embodiments the tunicate-derived ECM is derived from Polyclinum constellatum. In some embodiments, the tunicate-derived ECM is derived from Pallusia nigra.

In some embodiments, the biomaterial comprises tunicate-derived ECM and one or more additional ingredients or additives, such as additional natural or synthetic polymers, extracellular matrix proteins, extracellular matrix, natural or synthetic drugs, vitamins, proteins, growth factors, hormones, or the like. In some embodiments, the biomaterial comprises cells, which can be autologous, allogenic or xenogenic to an eventual recipient. In some embodiments, the biomaterial comprises neural stem cells, including neural stem cells derived from stem cells such as induced pluripotent stem cells. In some embodiments, the biomaterial comprises mesenchymal stem cells. In some embodiments, the biomaterial comprises fibroblasts.

In some aspects, the present invention provides biomaterials with components, such as collagen, derived from sustainable sources. In some embodiments, the biomaterial comprises collagen derived from sustainable sources, such as, but not limited to, barnacles, tunicates, fish, ECM components of marine organisms, and skin and shells from marine organisms. For example, in certain embodiments, the present invention provides biomaterials comprising fish-derived components such as collagen. In some embodiments, the biomaterial comprises collagen derived from the skin of a fish, such as from, but not limited to, grouper, carp, trout, or salmon. In some embodiments, the biomaterial comprises fish-derived collagen. In some embodiments, the biomaterial comprises decellularized fish-derived collagen. In some embodiments, the biomaterial comprises lyophilized fish-derived collagen. In some embodiments, the biomaterial comprises decellularized and lyophilized fish-derived collagen.

In some aspects, the present invention provides biomaterials with components derived from sustainable sources, such as, but not limited to, vegetables, fruits, plants and trees. In some embodiments, the biomaterial comprises components derived from fruits and/or vegetables. In some embodiments, the biomaterial comprises components derived from sustainable sources such as, but not limited to, kitchen wastes, vegetable wastes, and plant wastes. For example, in certain embodiments, the present invention provides biomaterials comprising banana-derived components such as cellulose. In some embodiments, the biomaterial comprises banana-derived components such as Cellulose Microfibers (CMFs). In some embodiments the biomaterial comprises cellulose derived from banana peels and/or stems. In some embodiments, the biomaterial comprises Cellulose Microfibers (CMFs) derived from banana peels and/or banana stems. For example, the biomaterial may comprise CMFs derived from banana stem of a banana tree or plant. In some embodiments the biomaterial comprises cellulose derived from watermelon and/or watermelon rinds. In some embodiments, the biomaterial comprises Cellulose Microfibers (CMFs) derived from watermelon and/or watermelon rinds. In some embodiments, the CMFs are coated in collagen derived from discarded fish skin.

The one or more components of sustainable sources, as described herein, can be used to produce various type of biomaterials, including, but not limited to, bioink, hydrogels, wound dressings, tunneling wound fillers, tissue engineered substrates, scaffolds, and the like.

In certain embodiments, the biomaterial comprises decellularized ECM. The ECM can be decellularized through one or more osmotic shock cycles. Osmotic shock cycles generally involve alternating exposure of ECM to a hypertonic solution and a hypotonic solution. An exemplary osmotic shock cycle comprises alternating between a hypertonic salt solution containing sodium chloride, mannitol, magnesium chloride, and potassium chloride, and a hypotonic solution containing 0.005% Triton X-100 in double distilled water for an hour incubation in each. In some embodiments, the hypotonic solution incubation can be performed under centrifugation. Further processing steps can include detergent washes, enzymatic digests, and organic solvent extraction, followed by the removal of all residual material using ion exchange beads.

The ECM can be prepared and decellularized to form decellularized ECM in any suitable manner (see U.S. Patent Application Publication No. 2011/0165676 and U.S. Pat. No. 9,814,802, which are each incorporated herein by reference in their entirety). For example, in various embodiments the ECM comprises tunicate-derived ECM that is decellularized into decellularized ECM (dECM).

The ECMs can be immersed in any suitable media, such as distilled water. The liquid media immersion permits the ECM to be homogenized uniformly. Each homogenization cycle comprises a homogenizing period with a resting period to permit the ECM solution to cool. The homogenizing step can be performed on ice to improve the rate of cooling. The homogenizing period can be between about 10 seconds and 1 minute, and the resting period can be between about 30 seconds and 5 minutes. In some embodiments, the homogenizing period is about 30 seconds and the resting period is about 120 seconds. In various embodiments, between about 10 to 100 homogenization cycles can be performed.

The biomaterials of the present invention can be shaped in any suitable manner. For example, in some embodiments, the biomaterial can be 3D printed into any desired size and shape. The biomaterial can be 3D printed with any suitable support structure, such as a casing or framework that is removable using commonly known post-processing steps. In other embodiments, the biomaterial can be shaped by being loaded into any sized mold. In some embodiments, the mold is selected to have a larger, nonspecific shape, such that the final molded biomaterial can be trimmed and resized to any desired shape. For example, in some embodiments, the biomaterial is formed by trimming and resizing native tunic-derived dECM. The shaped biomaterial is frozen at a temperature of −80° C. or below for at least 8 hours. The frozen biomaterial is then lyophilized at a temperature of about −20° C. and −60° C. at a vacuum of between about 0.01 mBar and 0.1 mBar for at least 8 hours.

In various embodiments, the biomaterial can be treated with a sterilization step. The sterilization step can apply any suitable sterilization method. For example, at any stage in the process of fabricating the biomaterial, the biomaterial components can be treated with radiation (e.g., gamma radiation, x-ray radiation, ultraviolet sterilization, and electron beam processing), gaseous formaldehyde, carbon dioxide, ozone, ethylene oxide, peracetic acid, ethanol, hydrogen peroxide, and the like.

In some embodiments, the biomaterials of the present invention can be enhanced with one or more additives. The additives can be mixed into a sample of homogenized biomaterial and can facilitate the adherence and growth of cells. For example, the one or more additives can include one or more additional extracellular matrix material and/or blends of naturally occurring extracellular matrix material, including but not limited to collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. In some embodiments, the biomaterial further comprises one or more surface treatments. In various embodiments, the one or more surface treatments can include one or more carbohydrates such as chitin, chitosan, alginic acids, and alginates such as calcium alginate and sodium alginate. In some embodiments, the surface treatments can include sucrose, fructose, cellulose, or mannitol. These materials may be isolated from plant products, humans or other organisms or cells, or synthetically manufactured.

In various embodiments, the additives can include natural peptides, such as glycyl-arginyl-glycyl-aspartyl-serine (GRGDS), arginylglycylaspartic acid (RGD), and amelogenin. In some embodiments, the additives can include nutrients, such as bovine serum albumin. In some embodiments, the additives can include vitamins, such as vitamin B2, vitamin Ad, Vitamin D, Vitamin E, and Vitamin K. In some embodiments, the additives can include nucleic acids, such as mRNA and DNA. In some embodiments, the additives can include natural or synthetic steroids and hormones, such as dexamethasone, hydrocortisone, estrogens, and its derivatives. In some embodiments, the additives can include growth factors, such as fibroblast growth factor (FGF), transforming growth factor beta (TGF-β), and epidermal growth factor (EGF). In some embodiments, the additives can include a delivery vehicle, such as nanoparticles, microparticles, liposomes, viral and non-viral transfection systems.

In various embodiments, the additives can include one or more therapeutics. The therapeutics can be natural or synthetic drugs, including but not limited to: analgesics, anesthetics, antifungals, antibiotics, anti-inflammatoirensteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

Aspects of the present invention relate to further additives, such as drugs, therapeutics, and the like, loaded into a biomaterial. Contemplated drugs or therapeutics include but are not limited to growth factors, neurotrophic factors, cell adhesion molecules, proteins, peptides, small molecules, nucleic acid molecules, cytokines, stem cells, Schwann cells, upregulators of regeneration-associated genes, conductive biocompatible materials, including but are not limited to polypyrrole (PPy), poly(3,4-ethylenedioxythiophene) polystyrene sulfonate (PEDOT: PSS), graphene, carbon nanotubes, metal nanoparticles, ionic liquids, and the like.

Exemplary growth factors or neurotrophic factors that can be embedded and released from the biomaterial include but are not limited to, glial cell derived neurotrophic factor (GDNF), nerve growth factor (NGF), epidermal growth factor (EGF), vascular endothelial growth factor (VEGF), fibroblast growth factor (FGF), ciliary neurotrophic factor (CNTF), platelet derived growth factor (PDGF), brain derived neurotrophic factor (BDNF), basic fibroblast growth factor (bFGF), neurotrophin 3 (NT-3), and neurotrophin 4 (NT-4), insulin-like growth factor 2 (IGF-2), and the like.

Aspects of the present invention relate to biomaterials comprising one or more cells. In some embodiments, the cells that can be cultured using the biomaterials of the present invention can be any suitable cell. Non-limiting examples of suitable cells include pluripotent stem cells, embryonic stems cells, hematopoietic stem cells, adipose derived stem cells, bone marrow derived stem cells, neural stem cells, mesenchymal stem cells, fibroblasts, osteocytes, epithelial cells, cardiomyocytes, endothelial cells, neurocytes, and the like.

In some embodiments, the biomaterial of the present invention is formed into at least one microparticle. Microparticles are generally understood by persons having skill in the art to refers to small particles which behave as a whole unit in terms of their transport and properties, and which typically exhibit an average particle size diameter (determined, for example, by a microscopy, electrozone sensing, or laser diffraction technique) in the range of about 0.1 to 10 μm or greater. Terms that may be used synonymously with microparticle include but are not limited to: nanoparticle, micro- and nanobubble, micelle, micro- and nanosphere, micro- and nanocapsule, micro- and nanobead, micro- and nanosome, and the like. Microparticles may comprise any structure suitable for the delivery of a desired therapeutic. For example, a microparticle may comprise a vesicle-like structure composed of a fluid core encased in a membrane comprising a lipid bilayer. Alternatively, a microparticle may comprise a hydrophilic shell and a hydrophobic core. A microparticle may also comprise one or more solid cores, or a distribution of solid or fluid deposits within a matrix.

The microparticles may be uncoated or coated to impart a charge or to alter lipophilicity. Microparticles may have a uniform shape, such as a sphere (e.g. a microsphere). Microparticles may also be irregular, crystalline, semi-crystalline, or amorphous. A single type of microparticle may be used, or mixtures of different types of microparticles may be used. If a mixture of microparticles is used they may be homogeneously or non-homogeneously distributed. In various aspects, the microparticle is biodegradable or non-biodegradable, or in a plurality of microparticles, combinations of biodegradable and non-biodegradable cores are contemplated.

In some embodiments, the microparticles comprise a polymer. Non-limiting examples of suitable polymers include but are not limited to PLGA, PLA, PGA, PCL, PLL, cellulose, poly(ethylene-co-vinyl acetate), polystyrene, polypropylene, dendrimer-based polymers, polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polysebacates, poly(glycerolsebacates), poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(l-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly(β-amino acids) (either homopolymers or random copolymers), poly(n-vinyl pyrrolidone) polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. For example, in some embodiments, the PLGA comprises any PLGA known in the art, including, but not limited to, 99:1 PLGA, 95:5 PLGA, 90:10 PLGA, 85:15 PLGA, 80:20 PLGA, 75:25 PLGA, 70:30 PLGA, 65:35 PLGA, 60:40 PLGA, 55:45 PLGA, 50:50 PLGA, 45:55 PLGA, 40:60 PLGA, 35:65 PLGA, 30:70 PLGA, 25:75 PLGA, 20:80 PLGA, 15:85 PLGA, 10:90 PLGA, 5:95 PLGA, and/or 1:99 PLGA.

In some embodiments, the microparticles are microspheres with at least one diameter of about 25 μm, 50 μm, 75 μm, 100 μm, 125 μm, 150 μm, 175 μm, 200 μm, 225 μm, 250 μm, 275 μm, or about 300 μm. For example, in some embodiments, the microspheres have a diameter of 125 μm. In some embodiments, the microspheres have different diameters.

In some embodiments, the biomaterial is or comprises a hydrogel. Hydrogels can generally absorb a great deal of fluid and, at equilibrium, typically are composed of 60-90% fluid and only 10-30% polymer. In a preferred embodiment, the water content of hydrogel is about 70-80%. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). The hydrogels may be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. (see.: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of hydrogels based on chemical or physical crosslinking synthetic polymers include but are not limited to (meth) acrylate-oligolactide-PEO-oligolactide-(meth) acrylate, poly(ethylene glycol) diacrylate (PEGDA), poly(ethylene glycol) (PEO), poly(propylene glycol) (PPO), PEO—PPO-PEO copolymers (Pluronics), poly(phosphazene), poly(methacrylates), poly(N-vinylpyrrolidone), PL (G) A-PEO-PL (G) A copolymers, poly(ethylene imine), etc. (see A. S Hoffman, 2002, Adv. Drug Del. Rev, 43, 3-12).

In one embodiment, the hydrogel comprises at least one biopolymer. In other embodiments, the hydrogel scaffold further comprises at least two biopolymers. In yet other embodiments, the hydrogel scaffold comprises at least one biopolymer and at least one synthetic polymer.

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels may also be made degradable in vivo by incorporating PLA, PLGA or PGA polymers. Moreover, hydrogels may be modified with fibronectin, laminin, vitronectin, or, for example, RGD for surface modification, which may promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes may influence strength, elasticity, and degradation properties of the instant hydrogels (Nguyen and West, 2002, Biomaterials 23 (22): 4307-14; Ifkovits and Burdick, 2007, Tissue Eng. 13 (10): 2369-85).

Hydrogels may also be modified with functional groups for covalently attaching a variety of proteins or compounds such as therapeutic agents. It is contemplated that linkage of the therapeutic agent to the matrix may be via a protease sensitive linker or other biodegradable linkage.

In certain embodiments, one or more multifunctional cross-linking agents may be utilized as reactive moieties that covalently link biopolymers or synthetic polymers. Such multifunctional cross-linking agents may include glutaraldehyde, genipin, epoxides (e.g., bis-oxiranes), oxidized dextran, p-azidobenzoyl hydrazide, N-[α.-maleimidoacetoxy] succinimide ester, p-azidophenyl glyoxal monohydrate, bis-[β-(4-azidosalicylamido)ethyl] disulfide, bis [sulfosuccinimidyl] suberate, dithiobis [succinimidyl proprionate, disuccinimidyl suberate, 1-ethyl-3-[3-dimethylaminopropyl] carbodiimide hydrochloride (EDC), N-hydroxysuccinimide (NHS) and other multifunctional cross-linking reagents known to those skilled in the art. It should be appreciated by those in skilled in the art that the mechanical properties of the hydrogel are greatly influenced by the cross-linking time and the amount of cross-linking agents. In some embodiments, the biomaterial comprises chitosan, and/or other cross-linking agents such as photoinitiators like Igracure, Riboflavin, and Lithium phenyl-2,4,6-trimethylbenzoylphosphinate (LAP), for photocrosslinking. In some embodiments, the biomaterial may include sodium alginate for calcium chloride ionic crosslinking or other chemical reagents for chemical crosslinking.

In another embodiment utilizing a cross-linking agent, polyacrylated materials, such as ethoxylated (20) trimethylpropane triacrylate, may be used as a non-specific photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as eosin Y, catalytic agents, such as 1-vinyl-2-pyrrolidinone, and triethanolamine. Continuous exposure of this reactive mixture to long-wavelength light (>498 nm) would produce a cross-linked hydrogel network.

In one embodiment, the hydrogel comprises a UV sensitive curing agent which initiates hydrogel polymerization. For example, in one embodiment, a hydrogel comprises the photoinitiator 4-(2-hydroxyethoxy)phenyl-(2-hydroxy-2-propyl) ketone. In one embodiment, polymerization is induced by 4-(2-hydroxyethoxy)phenyl-(2-hydroxy-2-propyl) ketone upon application of UV light. Other examples of UV sensitive curing agents include 2-hydroxy-2-methyl-1-phenylpropan-2-one, 4-(2-hydroxyethoxy)phenyl(2-hydroxy-2-phenyl-2-hydroxy-2-propyl) ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

The stabilized cross-linked hydrogel matrix of the present invention may be further stabilized and enhanced through the addition of one or more enhancing agents. By “enhancing agent” or “stabilizing agent” is intended any compound added to the hydrogel matrix, in addition to the high molecular weight components, that enhances the hydrogel matrix by providing further stability or functional advantages. Suitable enhancing agents, which are admixed with the high molecular weight components and dispersed within the hydrogel matrix, include many of the additives described earlier in connection with the thermo-reversible matrix discussed above. The enhancing agent may include any compound, especially polar compounds, that, when incorporated into the cross-linked hydrogel matrix, enhance the hydrogel matrix by providing further stability or functional advantages.

Exemplary enhancing agents for use with the stabilized cross-linked hydrogel matrix include polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine, and histidine. The preferred polar amino acids are L-cysteine, L-glutamic acid, L-lysine, and L-arginine. Suitable concentrations of each particular preferred enhancing agent are the same as noted above in connection with the thermo-reversible hydrogel matrix. Polar amino acids, EDTA, and mixtures thereof, are preferred enhancing agents. The enhancing agents may be added to the matrix composition before or during the crosslinking of the high molecular weight components.

### Methods of Preparation

In one embodiment, the present invention provides a method of producing a tunicate-derived ECM based biomaterial. In one embodiment, the method comprises obtaining a tunicate, decellularizing tunicate tissue, and lyophilizing tunicate tissue. In one embodiment, the method comprises powderizing the decellularized and lyophilized tunicate tissue and forming a solution comprising the powderized tissue to form a bioink. In one embodiment, the method comprises using a 3D printer and bioink to bioprint a hydrogel scaffold comprising the tunicate-derived ECM biomaterial. In one embodiment, the resultant biomaterial is then seeded with cells. In one embodiment, cells are added to the biomaterial prior to bioprinting. The tunicate-derived ECM based scaffolds described herein may be cultured in vitro or ex vivo to promote cell growth, proliferation, differentiation, and/or migration. In certain embodiments, the bioprinted hydrogel scaffold is crosslinked.

In some embodiments, the present invention provides a method of producing a tunicate-derived ECM based biomaterial. In some embodiments, the method comprises the steps of: dissolving tunic-derived dECM powder and Sodium Alginate (SA) in DMEM culture medium, and adding NaOH.

In some embodiments, the present invention provides a method of producing a sustainable biomaterial. In some embodiments, the method comprises the steps of: isolating an amount of Cellulose Microfibers (CMFs) from banana stem, powderizing the CMFs, stirring the CMFs with NaOH, filtering the CMFs, washing the CMFs, lyophilizing the CMFs.

In some embodiments, the present invention provides a method of producing a sustainable biomaterial. In some embodiments, the method comprises the steps of sing the steps of: isolating CMFs from banana stem, harvesting collagen from fish skin, coating the CMFs with the Collagen, and combining with Sodium Alginate.

### Applications/Methods of Use

Exemplary applications for the biomaterials described herein are wound-dressing materials, decellularized extra-cellular matrix (dECM) scaffolds for tissue engineering applications and bioinks for bioprinting of tissue constructs for regenerative medicine are potential applications. The versatility of the biomaterials allows their utility for different applications which includes but not limited to supporting different types of cells, co-culture of cells, engineered tissues, regenerative medicine, precision medicine, disease models, drug testing, in vitro tissue models for drug testing.

In one aspect, as described herein, the biomaterial is used for bioprinting of human Neural Stem Cells (hNSCs) and differentiating them into peripheral neurons post-bioprinting. In some embodiments, the biomaterial is optimized by adding Matrigel at a certain concentration to the biomaterial along with a certain concentration of sodium alginate and post-printing cross-linking with calcium chloride

In one aspect, as described herein, the biomaterial is used for bioprinting of human Mesenchymal Stem Cells (hMSCs) and differentiating them into chondrogenic and osteogenic lineages (cartilage and bone respectively) post-bioprinting. In some embodiments, the biomaterial was optimized by adding sodium alginate (commercially available) at a certain concentration to the biomaterial and post-printing cross-linking with calcium chloride

In some embodiments, the biomaterial can be used in vivo to promote the recruitment, infiltration, and differentiation of cells. The influx and maturation of cells into the biomaterial can be used to regenerate tissue to treat defects and wounds. Wounds for which the present inventive method is useful in promoting closure include, but are not limited to, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, or tangential wounds. In some embodiments, the biomaterial promotes ectodermal differentiation to regenerate the various substructures of the skin, including the sweat glands, sebaceous glands, hair follicles, and the like. The biomaterial may be secured to a wound area using sutures, adhesives, or overlaying bandages. The biomaterial may be cut to match the size of the wound, or may overlap the wound edges. In some instances the biomaterial may be shaped to penetrate into cavities formed by deep wounds. The biomaterial can also be used in mucosal injury healing, such as in surgery-related trauma and accidents.

In some embodiments, the biomaterial is applied cell-free, such that upon implantation, the biomaterial supports cell migration and proliferation from native tissue. The cell-free biomaterial can be supplemented with ECM and other cellular secretions to promote healing. In other embodiments, the biomaterial is seeded with one or more populations of cells to form an artificial tissue construct. The artificial tissue construct may be autologous, where the cell populations are derived from a patient's own tissue, or allogenic, where the cell populations are derived from another subject within the same species as the patient. The artificial organ construct may also be xenogenic, where the different cell populations are derived form a mammalian species that is different from the subject. For example the cells may be derived from organs of mammals such as humans, monkeys, dogs, cats, mice, rats, cows, horses, pigs, goats and sheep.

In some embodiments, the biomaterial is suitable for regenerating bone tissue and repairing bone defects. The biomaterial can be used as a scaffold for growth factor delivery to promote endogenous cell homing. Briefly, biomaterial can be sized to fit within a bone fracture or defect and loaded with osteogenic and angiogenic growth factors, including but not limited to bone morphogenetic protein (BMP-2) and vascular endothelial growth factor (VEGF). The site of a bone defect can be washed with saline prior to the transplantation of an appropriately sized biomaterial. The biomaterial is able to reduce or close a bone defect without inducing inflammation or an immunologic response. In some embodiments, the biomaterial is able to form new bone having typical bone morphology with noticeable marrow spaces similar to native bone.

### Cell Culture

In one aspect, the present invention encompasses methods for culturing cells. In various embodiments, the methods relate to the use of the biomaterial to support and expand one or more cell populations. The cells can be cultured in any suitable environment, including under in vivo and in vitro conditions. The cells that can be cultured using the biomaterial of the present invention can be any suitable cell. Non-limiting examples of suitable cells include pluripotent stem cells, embryonic stems cells, hematopoietic stem cells, adipose derived stem cells, bone marrow derived stem cells, neural stem cells, mesenchymal stem cells, fibroblasts, osteocytes, epithelial cells, cardiomyocytes, endothelial cells, neurocytes, and the like. Suitable cells can also include cancer cells, including but not limited to: leukemia, lymphoma, myeloma, breast cancer, prostate cancer, endometrial cancer, bladder cancer, brain cancer, cervical cancer, lung cancer, melanoma, cervical cancer, ovarian cancer, colorectal cancer, pancreatic cancer, esophageal cancer, kidney cancer, thyroid cancer, liver cancer, uterine cancer, soft tissue sarcoma, bone cancer, stomach cancer, and the like. In some embodiments, the biomaterial of the present invention maintain the plasticity of the cells that are seeded therein.

Cells may be isolated from a number of sources, including, for example, biopsies from living subjects and whole-organ recover from cadavers. The isolated cells can be autologous cells, obtained by biopsy from the subject intended to be the recipient. The biopsy may be obtained using a biopsy needle, a rapid action needle which makes the procedure quick and simple.

Cells may be isolated using techniques known to those skilled in the art. For example, the tissue may be disaggregated mechanically and/or treated with digestive enzymes and/or chelating agents that weaken the connections between neighboring cells making it possible to disperse the tissue into a suspension of individual cells without appreciable cell breakage. Enzymatic dissociation may be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption may also be accomplished by a number of methods including, but not limited to, scraping the surface of the tissue, the use of grinders, blenders, sieves, homogenizers, pressure cells, or sonicators.

Once the tissue has been reduced to a suspension of individual cells, the suspension may be fractionated into subpopulations from which the cells elements may be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for tissue reconstruction.

Isolated cells may be cultured in vitro to increase the number of cells available for seeding the biomaterial. The use of autologous cells can reduce or prevent tissue rejection typically seen with allogeneic cells. However, if an immunological response does occur in the subject after implantation of the artificial organ, the subject may be treated with immunosuppressive agents such as cyclosporin or FK506 to reduce the likelihood of rejection. In certain embodiments, chimeric cells, or cells from a transgenic animal, may be seeded onto the biomaterial.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chances of rejection by the host. In addition, transfection could also be used for gene delivery.

Seeded cells may be normal or genetically engineered to provide additional or normal function. Methods for genetically engineering cells with retroviral vectors, polyethylene glycol, or other methods known to those skilled in the art may be used. These include using expression vectors which transport and express nucleic acid molecules in the cells. (See Goeddel; Gene Expression Technology: Methods in Enzymology 185, Academic Press, San Diego, Calif. (1990). Vector DNA may be introduced into prokaryotic or cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

Seeding of cells onto the biomaterial may be performed according to standard methods. For example, the seeding of cells onto polymeric biomaterials for use in tissue repair has been reported (see, e.g., Atala, A. et al., J. Urol. 148 (2 Pt 2): 658-62 (1992); Atala, A., et al. J. Urol. 150 (2 Pt 2): 608-12 (1993)). Cells grown in culture may be trypsinized to separate the cells, and the separated cells may be seeded on biomaterial. Alternatively, cells obtained from cell culture may be lifted from a culture plate as a cell layer, and the cell layer may be directly seeded onto the biomaterial without prior separation of the cells.

In one embodiment, a range of 1 million to 50 million cells are suspended in medium and applied to each square centimeter of a surface of a biomaterial. The biomaterial is incubated under standard culturing conditions, such as, for example, 37° C. 5% CO2, for a period of time until the cells become attached. However, it will be appreciated that the density of cells seeded onto the biomaterial may be varied. For example, greater cell densities promote greater tissue regeneration by the seeded cells, while lesser densities may permit relatively greater regeneration of tissue by cells infiltrating the graft from the host. Other seeding techniques may also be used depending on the biomaterial and the cells. For example, the cells may be applied to the biomaterial by vacuum filtration. Selection of cell types, and seeding of cells onto a biomaterial, will be routine to one of ordinary skill in the art in light of the teachings herein.

In various embodiments, the biomaterial can be used in combination with different types of cells, tissues, and matrix materials to form complex tissues and organs for transplantation or in vitro drug testing. The biomaterial can be adapted for three dimensional printing to form the complex tissues and organs.

### High Throughput Screening

In one aspect, the present invention encompasses methods for high-throughput screening of any number of drugs and therapeutics using a biomaterial of the present invention. The biomaterial is highly reproducible and can be sized and shaped to fit any suitable high-throughput testing system. In some embodiments, the biomaterial can be used to support a high-throughput cell-based assay to screen the effectiveness of a drug or therapy.

The screening methods of the present invention are not limited to the specific type of the compound. Potential test compounds include chemical agents (such as toxins), pharmaceuticals, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the assays of the invention may screen a physical agent such as radiation (e.g. ionizing radiation, UV-light or heat); these can be tested alone or in combination with chemical and other agents. In one embodiment, entire compound libraries are screened. Compound libraries are a large collection of stored compounds utilized for high throughput screening. Compounds in a compound library can have no relation to one another, or alternatively have a common characteristic. For example, a hypothetical compound library may contain all known compounds known to bind to a specific binding region.

The assays of the invention may also be used to test delivery vehicles. These may be of any form, from conventional pharmaceutical formulations, to gene delivery vehicles. For example, the assays may be used to compare the effects of the same compound administered by two or more different delivery systems (e.g., a depot formulation and a controlled release formulation). They may also be used to investigate whether a particular vehicle could have effects by itself. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test compound may be a delivery vehicle of any appropriate type with or without any associated therapeutic agent. Non-limiting examples of delivery vehicles include polymersomes, vesicles, micelles, plasmid vectors, viral vectors, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Bioprinting of Bioactive Tissue Scaffolds from Ecologically-Destructive Fouling Tunicates

Urochordates are the closest invertebrate relative to humans and commonly referred to as tunicates, a name ascribed to their leathery outer “tunic”. The tunic is the outer covering of the organism which functions as the exoskeleton and is rich in carbohydrates and proteins. Invasive or fouling tunicates pose a great threat to the indigenous marine ecosystem and governments spend several hundred thousand dollars for tunicate management, considering the huge adverse economic impact it has on the shipping and fishing industries. In this work, the environmentally destructive colonizing tunicate species of Polyclinum constellatum was successfully identified in the coast of Abu Dhabi and methods of sustainably using it as wound-dressing materials, decellularized extra-cellular matrix (dECM) scaffolds for tissue engineering applications and bioinks for bioprinting of tissue constructs for regenerative medicine are proposed. The intricate three-dimensional nanofibrous cellulosic networks in the tunic remain intact even after the multi-step process of decellularization and lyophilization. The lyophilized dECM tunics possess excellent biocompatibility and remarkable tensile modulus of 3.85=0.93 MPa compared to ˜0.1-1 MPa of other hydrogel systems. This work demonstrates the use of lyophilized tunics as wound-dressing materials, having outperformed the commercial dressing materials with a capacity of absorbing 20 times its weight in the dry state. This work also demonstrates the biocompatibility of dECM scaffold and dECM-derived bioink (3D bioprinting with Mouse Embryonic Fibroblasts (MEFs)). Both dECM scaffolds and bioprinted dECM-based tissue constructs show enhanced metabolic activity and cell proliferation over time. Sustainable utilization of dECM-based biomaterials from ecologically-destructive fouling tunicates proposed in this work helps preserve the marine ecosystem, shipping and fishing industries worldwide, and mitigate the huge cost spent for tunicate management.

Tunicates or urochordates, commonly known as sea quirts are common marine invertebrates, with around 3000 different species (Bone, Q., Carre, C., & Chang, P. (2003). Journal of the Marine Biological Association of the United Kingdom, 83 (5), 907), including several tunicate classes such as Ascidiacea, Thaliacea, Larvacea and Appendicularia. Ascidians are the most studied class of tunicates comprising approximately 2300 species (Shenkar, N., & Swalla, B. J. (2011). PLOS One, 6 (6), e20657). Despite their lack of a spinal cord, tunicates are one of the few invertebrates in the phylum Chordata which makes them of interest to evolutionary scientists as the closest invertebrate relative to humans (Delsuc, F., Brinkmann, H., Chourrout, D., & Philippe, H. (2006). Nature, 439 (7079), 965-968c, Brinkmann, Chourrout, & Philippe, 2006). In the larval stage, ascidian tunicates are pelagic tadpole like swimming creatures, however, once adulthood commences, they attach to a solid surface such as rocks or ship hulls and metamorphize into a benthic sac-like body as their tail resorbs providing food reserves for the animal (Goodbody, I. (1975). Advances in marine biology, 12, 1-149). Adult tunicates mainly operate as filter feeders (Lambert, G. (2001). The biology of ascidians, 249-257 2001); filtering as small as 10 μm of particles Deibel, D., & Powell, C. (1987). Marine Ecology Progress Series, 243-250). This function is optimized by their structure which externally constitutes a thick protective tunic, with inhalant and exhalant siphons for water flow, while the interior is largely comprised of a branchial basket covered by a mucosal mesh filter constructed in the endostyle (Di Bella et al. (1998). Tissue and Cell, 30 (3), 352-359).

Several ecological, economical, and public health hazards are associated with tunicates. Tunicates are ‘invaders’ that travel from one region or port to other by attaching to the ship bottoms (Therriault et al. (2008). Canadian Science Advisory Secretariat). This is referred to as ‘vessel fouling’, which has a huge economic impact on the shipping and fishing industries. The tunicate removal process involves injecting high-pressure water or lime solutions, desiccation and asphyxiation, which is a costly procedure and requires expensive equipment (Locke et al. (2009). Aquatic Invasions, 4 (1), 249-258). The state of Washington in the United States spent $750,000 in 2006 and 2007 for tunicate management at Puget Sound (Pleus et al. (2008). Department of Fish and Wildlife). Besides this huge economic impact, tunicates also pose a huge threat to marine ecology. As a consequence of their filtration dominant survival techniques, tunicates compete for food with other filter feeders such as clams, mussels and scallops. However, due to their high reproduction rate and temperature and salinity tolerance they can quickly replace such native species by overgrowing and taking over an area, making them a major threat to biodiversity (Dunlop, M. J., Acharya, B., & Bissessur, R. (2018). Journal of Environmental Chemical Engineering, 6 (4), 4408-4412).

Harvesting the invasive tunicates for extraction of useful biomaterials offers a potential solution. The tunic, which is the thick external skin from which the organism derives its name, is mainly composed of tunicin, a cellulose polysaccharide, in addition to some collagen and elastin which act as a skeletal support structure, as well as the tunicate's first line of defense (Franchi, N., & Ballarin, L. (2017). Frontiers in immunology, 8, 674). Most of the published literature on utilization of tunics deal with the extraction of cellulose and cellulose nanofibres. Zhao et al. (Zhao, Y., & Li, J. (2014). Cellulose, 21 (5), 3427-3441) extracted the tunic cellulose (TC) from four different species, with variations in chemical and morphological structures between the species. The same group (Zhao et al. (2015). Carbohydrate polymers, 117, 286-296) reported the extraction and structure-property relationships of tunicate cellulose nanofibers (T-CNFs). Similar works on structure-property relationships of T-CNFs (Moon et al. (2021). Carbohydrate polymers, 254, 117470) and enhanced mechanical strength of structures with addition of tunicate cellulose nanocrystals in rubber (Wang et al. (2021). Journal of Materials Chemistry C, 9 (19), 6344-6350) and polymeric networks (Hu et al. (2020). Composites Part B: Engineering, 197, 108118) were reported. While extraction of cellulose and nano-cellulose from tunicates are good, the greatest drawback is that the yield is only 5% (Dunlop et al. (2020). Scientific reports, 10 (1), 1-13), leaving the rest of the 95% as waste. The intricate 3D structure of the cellulosic nanofibers is not utilized and the presence of other proteins and polysaccharides in the dECM that might be useful for cell growth and differentiation is lost in the process.

In this work, the native tunicate extra-cellular matrix, after decellularization and lyophilization, were used in three different ways: (i) as bioactive tissue engineering scaffold (ii) wound-dressing material and (iii) formulation of bioink for bioprinting. There are multi-fold benefits of this novel approach such as the utilization of the nanofibrous cellulose network for cell attachment, growth, and proliferation (unlike the cellulose extraction process which damages the network), retaining the high-water-absorbing capacity of the native tunic, and minimizing the waste and use of toxic chemicals in the cellulose extraction process. The lyophilized tunicate ECM has several unique properties. It has a high water-absorption capability and resumes its original shape and weight when resuspended in water or cell culture media. This not only ensures continuous supply of the nutrients and other growth factors to the cells suspended in its three-dimensional polymeric network but also offers the advantage of ease of handling/shipping a dry 3D scaffold compared to 3D hydrogel systems. Sustainable utilization of marine tunicates into highly valuable marine biomaterials using the approaches proposed in this work will help overcome major issues posed by these invasive organisms, thus preserving the marine ecosystem from the ecologically-destructive tunicates in the coastal areas. This approach could be considered as an excellent and efficient alternative for marine tunicates management in a sustainable way.

First, the identification and detailed morphological characterization of the species is presented, followed by material and mechanical characterization of the as-harvested, decellularized and lyophilized tunics. Next, three different applications are described, namely dECM scaffold, dECM-based bioink for bioprinting and wound-dressing material.

### Materials and Methods

### Materials

Tunicates were collected from the Zayed Port, Abu Dhabi, UAE. The samples were thoroughly washed with DI water in continuous stirring and stored in 90% ethanol for species identification. The species was identified as Polyclinum Constellatum and submitted in NCBI (Accession #MW990087). Camel blood samples were collected from the Al Wathba animal market, Abu Dhabi.

Decellularized Extra Cellular Matrix Scaffold (dECM) Preparation

The outer rough layer was removed using surgical knife and the whole hydrogel-like tunic tissue was separated from the freshly harvested Polyclinum constellatum. DI water was extensively used to clean the soft tissue several times at room temperature, before being cut into required dimensions. The tunic tissue pieces are stirred well in decellularization buffer (10 mM Tris, 1 mM of ethylenediamine tetra acetic acid (EDTA), 0.2% V/V of Triton X-100, and 1.5% of sodium dodecyl sulfate (SDS); pH 7.5 all from Sigma-Aldrich, USA) for 48 hours. The buffer was changed every 2 hours. DI water was used to remove any cellular debris. The decellularized tissue pieces were frozen in −80° C. overnight and lyophilized (Christ Alpha 1-2 LD Lyophilizer) for 48 hrs. The lyophilized scaffolds were sterilized with ethanol and UV radiation for further characterization and analysis.

### Material Characterization

The microstructure of the tunics was imaged using Quanta™ 450 FEG SEM. The morphology of the tunics was imaged using tapping-mode Agilent 5500 AFM equipped with a multipurpose scanner, 90 μm, 670 nm low coherence Triple lock-in AC mode controller. The AFM images were recorded under ambient conditions with 50% relative humidity at 23° C. RTESP silica cantilevers having a tip with a radius of 8 nm and a spring constant of 40 N/m oscillated at their fundamental resonance frequencies between 200 and 400 kHz. Agilent 670-FTIR was used for the FT-IT analysis, with the absorption and transmission spectra measured for each position in the 400-4000 cm-1 region with 0.5 cm-1 resolution and precision of wavenumber definition not less than 0.01 cm-1. Thermogravimetric analysis (TGA) was carried out using SDT Q600 Instrument, in air atmosphere. Malvern Panalytical Empyrean 3 advanced instrument was used for powder XRD, conducted at room temperature from 2-400 (20). Raman spectroscopy was performed using WITec alpha 300 equipment, with spectra recorded from 0-3500 using 600 mW laser. Uniaxial tensile test was carried out using Instron UTS-5965 with a load cell of 500 N and a crosshead speed of 40 mm/min.

### Cell Culture

Mouse Embryonic Fibroblasts (MEFs) cells were cultured in complete media (DMEM supplemented with 10% fetal bovine serum, 1% penicillin-streptomycin and 2 mM L-glutamine) for proliferation studies and subsequently cultured in flasks and maintained in an incubator (5% CO₂, 37° C., and humidified atmosphere). The cells were trypsinized at 70% confluency and sub-cultured further before seeding them on lyophilized tunics. The lyophilized tunics were sterilized using 70% ethanol overnight in a laminar airflow chamber followed by UV sterilization for an hour in 24 well plates. Each scaffold was seeded with 200,000 cells for all the experiments.

### Bioinks Preparation

100 mg of dECM powder was mixed with the 0.2% sodium alginate (SA) (Spectrum® Chemical MGF.CORP. Gardena, CA) dissolved in 1.5 ml of DMEM culture medium for the preparation of bioink. The hydrogel was mixed and gently stirred for one hour at 500 rpm at 37° C., without air bubbles, before adding 1.5 M NaOH (Sigma, USA) to adjust the pH to 7. All the processes were performed under sterile conditions. The developed bioinks were stored at 4-8° C. and the temperature was raised to 37° C. before bioprinting.

### 3D Bioprinting

A regenHU 3D-Discovery™ Bioprinter (regenHU Ltd, Switzerland) was used for bioprinting of tissue constructs. MEFs were cultured per the standard protocol and re-suspended in dECM/SA solution (100 mg of dECM/0.2% SA in 1.5 ml of DMEM cell culture media) to maintain a final concentration 200,000 cells/construct. Bioink was loaded into an extrusion cartridge; the bioprinting was done with a nozzle of 0.1 mm diameter and pressure 0.5-0.6 MPa. After successful printing, the bioprinted constructs were crosslinked with 200 mM CaCl₂) for five minutes and further incubated with 0.2% FBS for 15 minutes. The bioprinted constructs were then washed gently with PBS and incubated in the standard culture media (DMEM with 10% FBS and 1% penicillin-streptomycin) for further characterization.

### Cell Viability and Proliferation

To quantitatively analyse the metabolic activity of MEFs grown on P. constellatum derived dECM scaffold and bioprinted tissue constructs, Alamar Blue (AB) assay (Bio Source International, Camarillo, CA, USA) was performed per manufacturer's protocol. Briefly, both cell-seeded dECM scaffolds and bioprinted constructs were incubated for 4 h in 0.5 ml media in 24 well plates with 10% AB solution prepared in cell culture medium. After incubation, the reduced solution was transferred to a 96-well plate, absorbance was recorded at 570 nm and 600 nm by a microplate reader (Biotek Synergy H1, USA) and the percentage of dye reduction was calculated. The negative control was cell-free scaffolds. The viability of MEFs within dECM scaffold and bioprinted constructs was also evaluated by LIVE/DEAD staining with Calcein AM and Ethidium homodimer1 (LIVE/DEAD™ Viability/Cytotoxicity Kit, Thermo Fisher Scientific, USA). Briefly, the dECM and 3D-bioprinted constructs were gently washed with PBS before being stained with 500 μL of 2M Calcein AM and 4M Ethidium homodimer1 working solution for one hour at 37° C. incubator. After incubation, the scaffolds were mounted on a clean slide for imaging using Leica SP8 confocal laser-scanning microscope. The MEFs cell proliferation was evaluated by Quanti-iT™ PicoGreen™ ds DNA Assay Kit (Invitrogen, ThermoFisher Scientific, USA) by measuring the quantity of dsDNA on days 1,3,5, and 7 respectively as per the manufacturer's protocol. The scaffolds were treated with lysis buffer (10 mM Tris-HCl, 1 mM EDTA, pH 7.5). Subsequently, 100 μl of pico-green (Molecular Probes, Invitrogen GmbH, Karlsruhe, Germany) at 200× dilution in TE buffer was added to 50 μl of the sample and incubated for 5 minutes at room temperature in dark. Fluorescence was measured at an excitation and emission wavelength of 485 nm and 520 nm using a microplate reader (Biotek Synergy H1, USA). Metabolic activity of the samples was investigated on days 1 and 7, respectively. Cell-free scaffolds served as negative control. The total protein content of the dECM scaffold was quantified on days 1, 3, 5 and 7 using the BCA assay. The scaffolds were treated with lysis buffer (10 mM Tris-HCl, 1 mM EDTA, pH 7.5; all from Pierce™ BCA Protein Assay Kit, Thermo Scientific, USA); 25 μl of the lysate was added into 96-well cell culture plate followed by the addition of 200 μl of BCA working solution to it and incubated at 37° C. for 1 hour. The absorbance was measured at 562 nm using a microplate reader (Biotek Synergy H1, USA).

### Exudate Absorption Studies

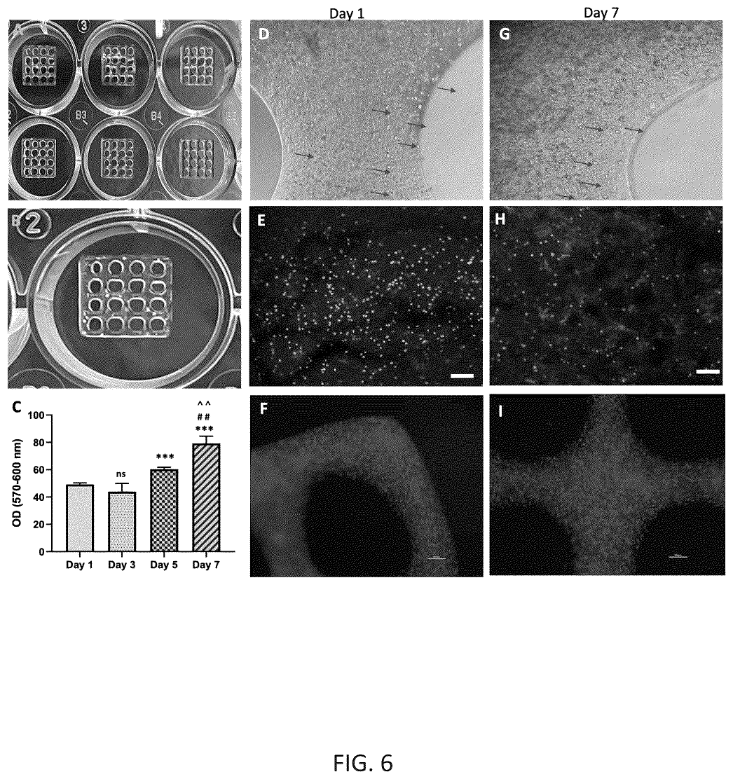

40 mL of whole camel blood in an appropriate collection tube was centrifuged at 3000 rpm at 8° C. for 15 minutes. The supernatant plasma was pipetted into a clean plastic screw-cap vial for further experiments. For the exudate absorption study, the lyophilized tunics were immersed in camel blood plasma (with diluted alizarin red solution), at predetermined time intervals (0M, 30S, 1M, 3M, 5M, 10M, 15M, 30M, 1 Hr, 6 Hr, 12 Hr, 24 Hr and 48), the tunics were removed from the plasma solution, weighed, and kept back in the plasma solution. To study the potential of the tunic as a wound-dressing material, alginate-based artificial wound model (FIG. 6B) was used, with camel blood plasma (with diluted alizarin red solution) as the exudate. The fluid uptake and distribution in the materials were photographed. Commercially available dressing materials such as Cotton Cloth, Gazin®, and Euromed® were used for comparison.

### Results and Discussion

### Identification of Fouling Tunicates—a Marine Hazard

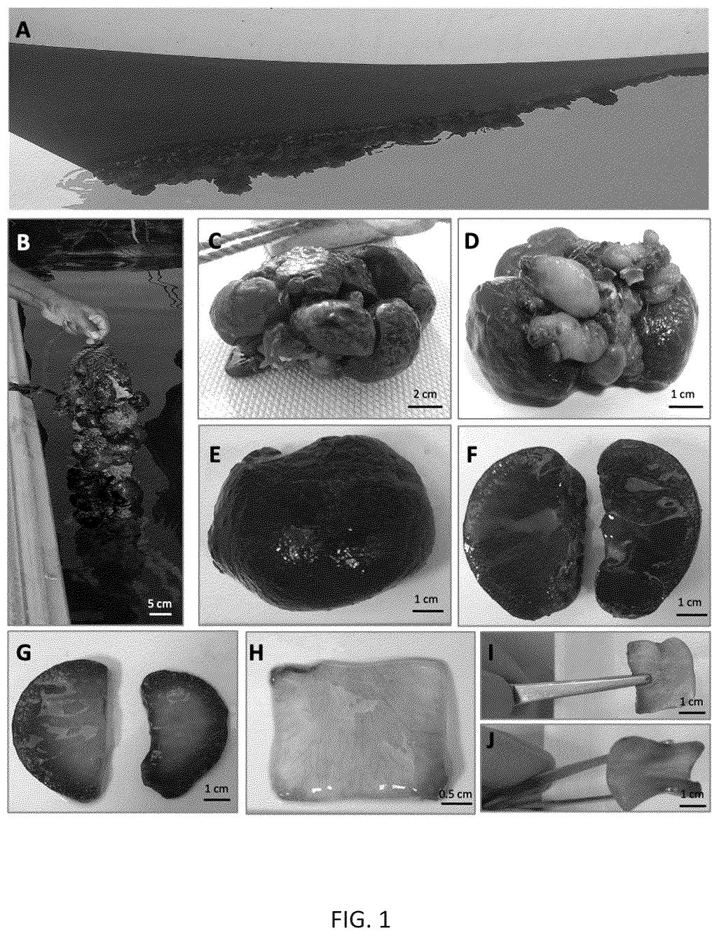

The tunicate species was identified to be Polyclinum constellatum (NCBI Accession #MW990087). The species belong to the non-indigenous colonial fouling tunicates (ÖNEN, 2018), which are ecologically destructive to the marine environment (Streit et al. Marine Pollution Bulletin, 167, 112262). The species colonizes the harbor areas, growing on the critical parts of the ship, including the hull, bulbous bow, propeller, and various places and structures in the port such as the wharf, dock, quay, and pier (FIG. 1A & 1B). Removal of the tunicate colonies from the ships, boats and the port structures take time and money, also damaging the structures in the removal process. The species identified in the coast of Abu Dhabi showed similar morphological features as other Polyclinum constellatum species reported elsewhere (Streit et al. Marine Pollution Bulletin, 167, 112262). Tunicate species with various color morphs including brown, red, green, and honey-colored were identified-all belonging to the same species (FIGS. 1C-E). This species has a jelly-like tunic consistency externally (FIG. 1E) and more so internally (FIGS. 1F-G). Predominantly made of cellulose, the cellulose fibrils are visible on slicing the top layers of the tunic (FIG. 1H). The tunic, as such, also was very flexible and easy to handle (FIG. 1I-J).

Tunic Possesses a Rough Multi-Layered Networked Structure with Visible Nano-Fibrillar Cellulose Networks

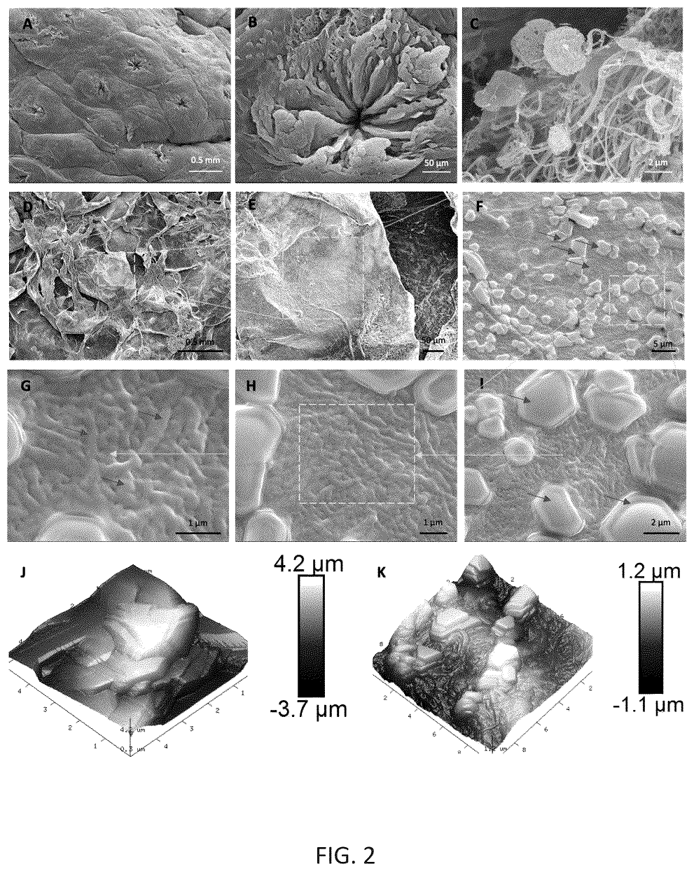

The external morphology of the tunic both in as-harvested condition and after decellularization and lyophilization was investigated. FIGS. 2A-C show the external morphology of as-harvested tunicate surface. The external surface of the tunicate (FIG. 2A) resembles the spiny eyes on the surface of the pineapple; hence the organisms are also being referred to as sea pineapples [Song, G., et al., Structure and composition of the tunic in the sea pineapple Halocynthia roretzi: A complex cellulosic composite biomaterial. Acta Biomater, 2020. 111: p. 290-301]. A magnified image of the ‘eye’ (FIG. 2B) reveals the pores through which the organism intakes and exudes water, with the phytoplanktons and zooplanktons seen closer to the pores (FIG. 2C). Growing in colonies and in large numbers, this species poses a threat to the indigenous marine organisms depriving of their food (i.e.) phytoplanktons and zooplanktons. The tunics were decellularized and lyophilized before further processing. The external morphology of a lyophilized tunic is shown in FIGS. 2D-I. The SEM images reveal that the lyophilized tunic has a rough multi-layered networked structure, with the presence of micro/nano-fibrils and crystals (FIGS. 2F-I). It is interesting to note that the nano-fibrillar cellulose networks remain intact even after the process of decellularization and lyophilization. The genetic material is removed as a result of decellularization and the cellulose networks are densified after lyophilization after the removal of water. While this work is the first to report on the detailed morphological characterization of Polyclinum constellatum, the results are consistent with the previous reports on the morphology of a different tunicate species Halocynthia roretzi (Hirose, E., Ohtake, S. I., & Azumi, K. (2009). Journal of fish diseases, 32 (5), 433-445). Representative topological images obtained by AFM (FIGS. 2J-K) reveals the tunic surface to be uneven and oblique. The negative sign represents the areas that lie below the baseline surface. Since tunics are natural tissues unlike engineered tissues, their surfaces are naturally uneven. The presence of high-aspect ratio crystal-like cellulose and dense nano-fibrillar cellulose can be clearly seen, with varying roughness depending on the nature of the tunic and composition of the crystal-like and nanofibrillar structures. The nanofibrous cellulose networks provide the necessary mechanical properties such as the tensile modulus and structural integrity (Wagner, R., Moon, R. J., & Raman, A. (2016). Cellulose, 23 (2), 1031-1041).

### Material Characterization of the Tunic Confirms the Presence of Cellulose

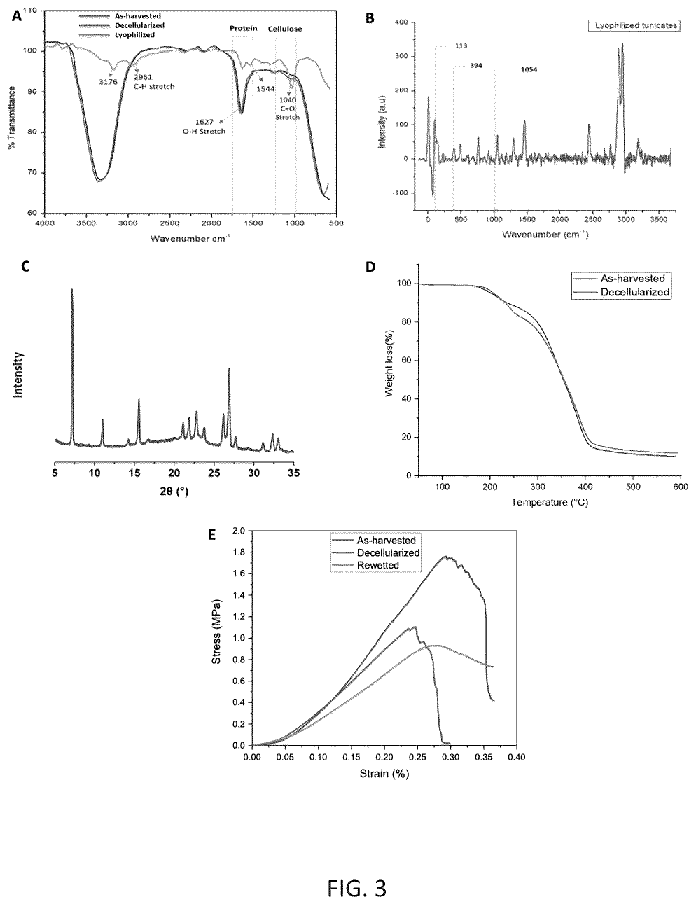

The FT-IR spectra of as-harvested, decellularized, and lyophilized tunic (FIG. 3A) indicates the presence of cellulose and proteins, the main constituents of the tunicate. The overlapped bands of as-harvested and decellularized tunics at 3336 cm⁻¹ and 1627 cm⁻¹ represents the O—H stretching, which is absent in lyophilized tunic (absence of O—H groups due to water removal) (Zhao, Y., & Li, J. (2014). Cellulose, 21 (5), 3427-3441). The strong bands at 1627, 1544, and 1296 cm-1 represent amide I, II, and III bands respectively, indicating the presence of secondary structures of the protein backbone (Movasaghi et al. (2008). Applied Spectroscopy Reviews, 43 (2), 134-179). The sharp peak at 1040 cm-1 representing the bending frequencies of the C—OH groups of carbohydrates is the main characteristic peak indicating the presence of cellulose (Song et al. (2020). Acta Biomaterialia, 111, 290-301). The FT-IR spectra of Polyclinum constellatum are remarkably similar to another tunicate species Ciona intestinalis reported previously (Nakashima, K., Sugiyama, J., & Satoh, N. (2008). Marine genomics, 1 (1), 9-14), proving the presence of cellulose in different tunicate species. The presence of cellulose is also confirmed by Raman spectra (FIG. 3B) by the peaks at 1054, 394, and 113 cm-1 (Agarwal, U. P., Reiner, R. S., & Ralph, S. A. (2010). Cellulose, 17 (4), 721-733). X-ray diffractogram of the powdered (decellularized) lyophilized tunic (FIG. 3C) further confirms that the tunic cellulose is highly crystalline. The characteristic peaks of tunicate cellulose at 2θ=22.9°, 16.6°, and 14.8°, with the peak at 22.9º attributed to diffraction (200) and the other two peaks at 16.6° and 14.8º attributed to the reflections, are in good agreement with the previous studies on tunicate cellulose (Kale et al. (2018). Journal of Polymers and the Environment, 26 (1), 355-364).

The TGA thermograms of as-harvested and decellularized tunic, both after lyophilization, are shown in FIG. 3D. The onset temperature of both as-harvested and decellularized tunic is around 200° C., which agrees with the TGA thermogram range of tunicate-derived cellulose/cellulose nanocrystals reported in the literature (Jun et al. (2020). Biotechnology & Bioprocess Engineering, 25 (2)). The ash content also does not vary significantly (less than 2%). More than 70% weight loss occurs in the temperature range of 250-400° C., which is well within the temperature range of 280 to 400° C. associated with the thermal decomposition of cellulose (Moon et al. (2011). Chemical Society Reviews, 40 (7), 3941-3994). The decomposition is complete at around 400° C. for both the decellularized and the as-harvested tunic. There was a slight difference (decomposition temperature extended by 20-30° C. for as-harvested samples) in different samples analyzed due to the presence of inorganic salts and heavy metal ions in the as-harvested tunicates that they pick up from the marine environment as they are suspension feeders (Zhao, Y., & Li, J. (2014). Cellulose, 21 (5), 3427-3441).

Lyophilized dECM Shows Remarkable Tensile Modulus of 3.85±0.93 MPa Compared to ˜0.1-1 MPa of Other Hydrogel Systems

Mechanical properties and structural stability are of critical importance in tissue engineering. The main challenges associated with soft tissue engineering (predominantly using hydrogels) are poor structural stability and weak mechanical properties. The stress-strain curves obtained from the uniaxial tensile test of as-harvested, decellularized, and rewetted tunics are shown in FIG. 3E. The stress-strain curves have a non-linear profile and are J-shaped, typical of the biological soft tissues (Xu et al. (2013). Journal of the mechanical behavior of biomedical materials, 28, 354-365). The peak stress was highest in the as-harvested tunic (1.80±0.07 MPa), compared to the decellularized (1.04±0.08 MPa) and rewetted (0.833±0.13 MPa) tunics, which might be due to the presence of cells and other genetic material in the as-harvested tunic. The elastic modulus of the tunics was in the range of 7.49±0.28 MPa for as-harvested, 5.1±1.11 MPa for decellularized, and 3.85±0.93 MPa for the rewetted tunics. The tunicate has the same behavior as a hydrogel but with much higher mechanical strength. The tunic derived in this work overcomes the most critical weakness of the hydrogel which is its extreme softness. The modulus of most hydrogels is in the range of 0.1 MPa (Vijayavenkataraman et al. (2019). International Journal of Bioprinting, 5 (2.1)) and researchers were enhancing the modulus of the hydrogel by various additives to bring the modulus to above 1 MPa (Zhu, L., Qiu, J., & Sakai, E. (2017). RSC advances, 7 (69), 43755-4376). Tunic, being a natural ECM material, has a modulus of 4 MPa, which is much higher than other hydrogels reported so far in the literature. The tunic, derived in this work, combines both excellent biocompatibility and mechanical strength, overcoming the limitations of most of the hydrogel-based constructs. It is important also to mention that the wavy end of the stress-strain curve is due to the slipping of the tunic samples at the end-grippers, while the tunic isn't damaged. The slippage is due to the jelly-like nature of the tunic, releasing the absorbed water as the specimen is pulled apart. A stronger grip led to stress-concentration in the specimen, damaging the specimen at the area where it is gripped.

### High Fluid-Retaining Capacity, Capillary-Like Behavior of the 3D Nanofibrous Cellulosic Networks and Shape-Retaining Capability—a Novel and Unique 3D Scaffold

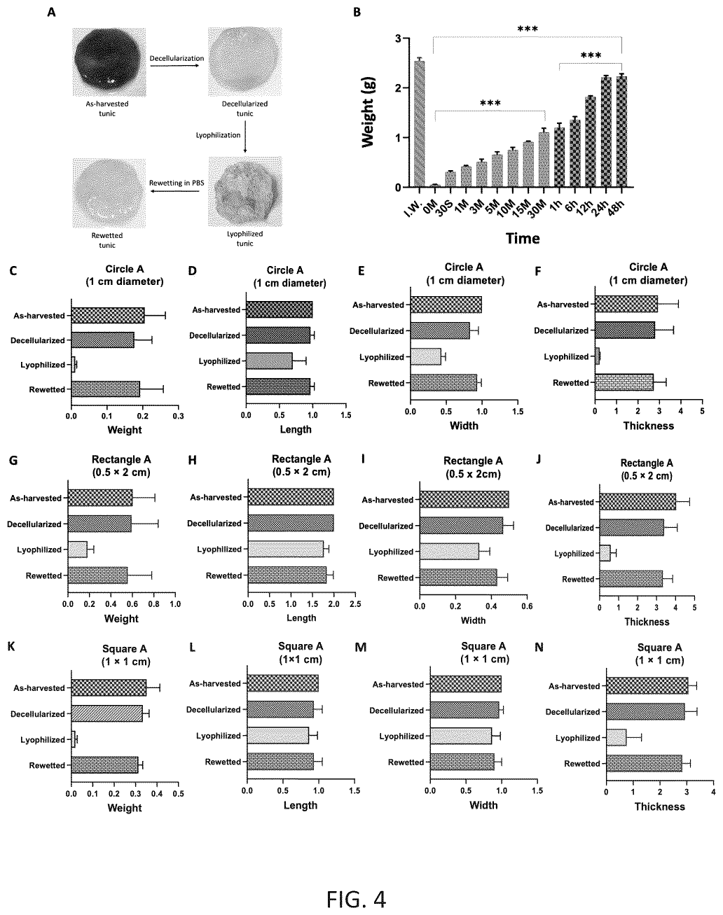

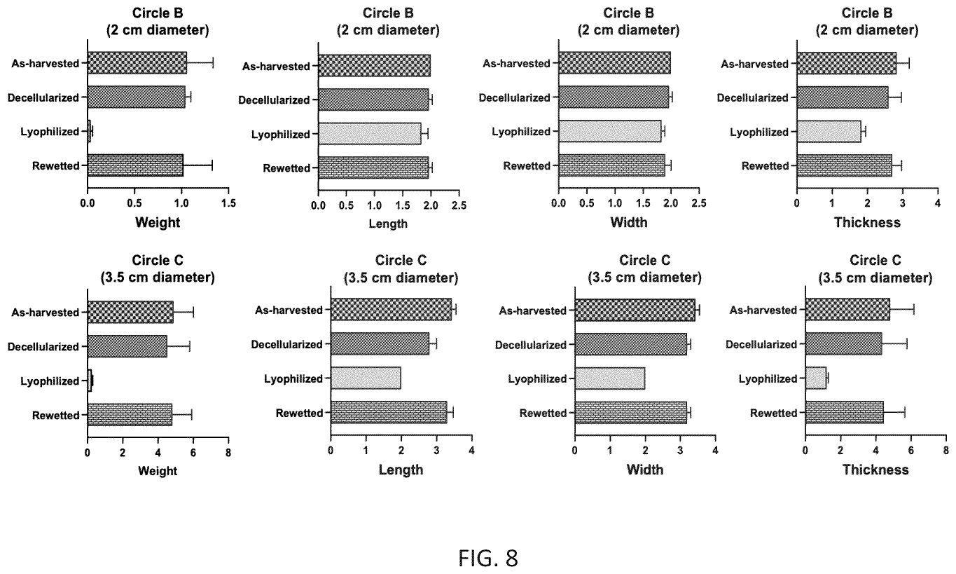

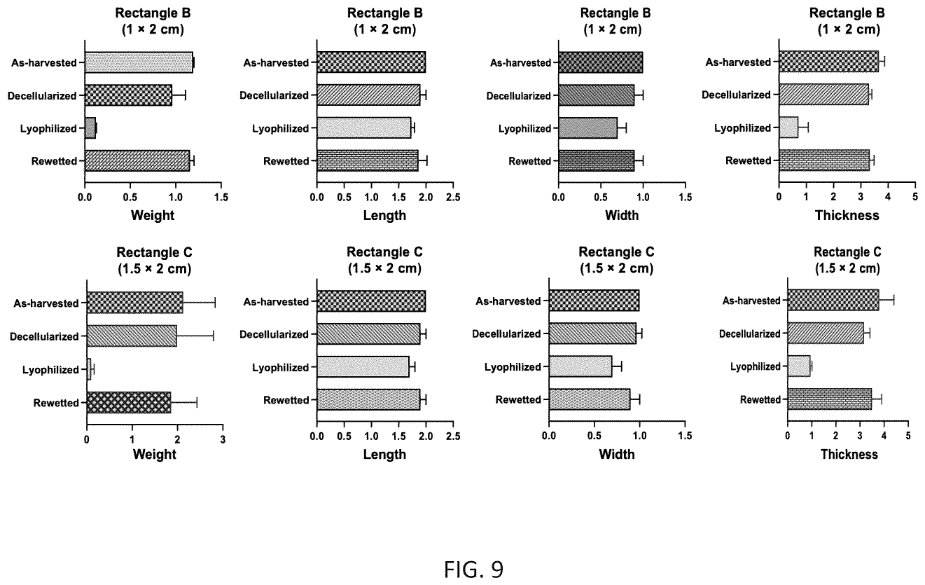

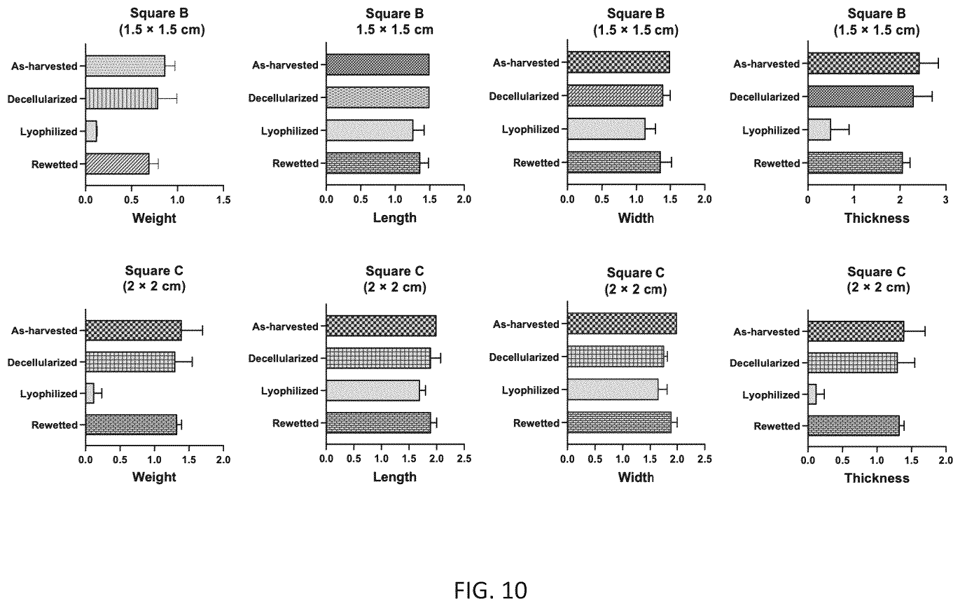

The most interesting observation from the present work is the ability of the tunic to retain its nanofibrous cellulose networks intact even after the multi-step process of decellularization and lyophilization. It is also important to note that the external morphology of the tunic was also retained throughout the process. FIG. 4A shows the external morphology of a tunic, cut in the shape of a circle of diameter 1 cm, indicating the full cycle of the process, starting from the as-harvested sample to rewetting of the sample with PBS. While there is a great reduction of thickness due to lyophilization, the diameter did not vary significantly. The lyophilized sample, on rewetting, came back to its former self, both in terms of its weight and dimensions. The swelling behavior of the lyophilized tunic (FIG. 4B) was extra-ordinary due to the inherent nature of the tunic and the three-dimensional cellulose networks that has a very high fluid-absorbing and retaining capabilities. A lyophilized tunic of weight 0.051 g significantly absorbed the fluid (PBS) and gained a weight of 1.102 g within 30 minutes. The weight continued to increase gradually until the weight is saturated at 2.23 g after 24 hours, compared to its pre-lyophilized weight of 2.56 g. The results are highly encouraging, proving the capability of the lyophilized tunics to be used as tissue engineering scaffolds and drug delivery substrates. The high fluid-retaining capacity and capillary-like behavior of the 3D nanofibrous cellulosic networks is expected to provide a favorable environment to the cells, in terms of cell infiltration, migration, growth, diffusion of cell nutrients and growth factors, and removal of waste (Heise et al. (2021). Advanced Materials, 33 (3), 2004349). The fact that the lyophilized tunics are dry and can be easily transported compared to other 3D culture systems such as hydrogels and on rewetting with PBS or culture media, the 3D tunic structure is regained, is a huge advantage for labs around the world trying to establish sustainable 3D culture systems. For example, the as-harvested tunics, after initial cleaning, can be cut into the desired shapes such as the size of different well-plates (24, 48, or 96-well plate diameters) and then decellularized and lyophilized as a batch. To enable this process flow, it is important to evaluate the shape-retaining capability of the tunics before and after lyophilization, with various possible shapes. Three basic shapes were considered as a proof-of-concept demonstration, namely circle, rectangle, and square, with three different dimensions in each. Circle A, B, and C with diameters of 1 cm, 2 cm, and 3.5 cm respectively, rectangular samples A, B, and C with dimensions of 0.5×2, 1×2, and 1.5×2 cm respectively and square sections A, B, and C of sides 1, 1.5, and 2 cm respectively were considered. The representative results of Circle A (FIGS. 4C-F), rectangle A (FIGS. 4G-J), and square A (FIGS. 4K-N), in terms of its weight and dimensions are given in FIG. 4. The results of the rest of the samples (circles B & C, rectangles B & C, and squares B & C) are given in FIG. 8, FIG. 9, and FIG. 10. It can be clearly seen from the results that irrespective of the external morphology, the overall shape and dimensions remain intact after decellularization and lyophilization, with only the weight and thickness reduced significantly due the removal of water during the lyophilization process. On rewetting, the lyophilized samples regain their weight and thickness, closer to its pre-lyophilized values.

The dECM-Based Tunic Scaffolds and dECM-Derived Tunic Bioink Show Excellent Biocompatibility—Bioprinting with Tunic Bioink

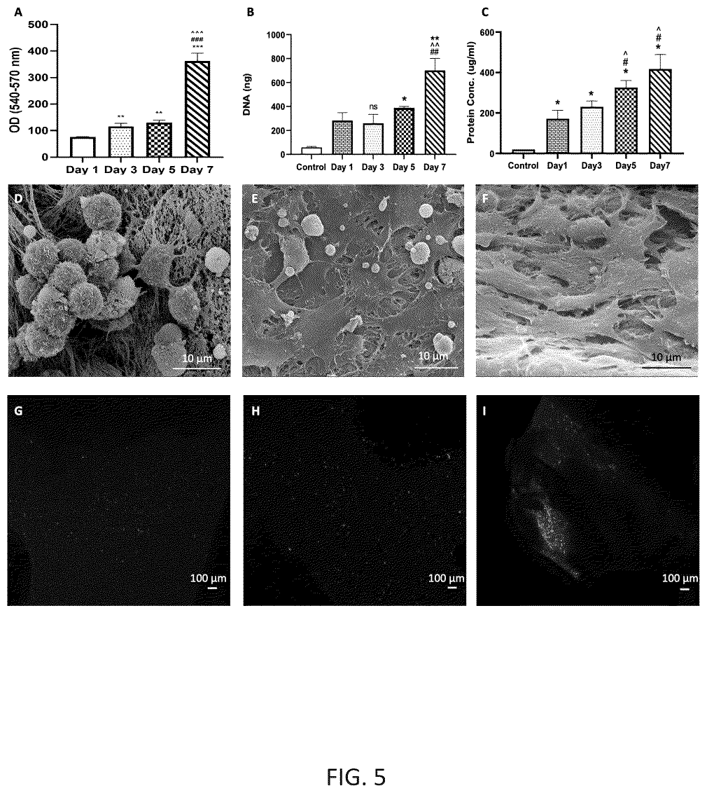

Cell culture studies are important to evaluate the cytotoxicity of the natural biomaterials and to prove that the preparation process has no adverse impact on the attachment, growth, and proliferation of cells. The rewetted tunics were seeded with Mouse Embryonic Fibroblasts (MEFs). Also, dECM-based bioinks were prepared for bioprinting and tissue constructs were bioprinted with MEFs suspended in the dECM-alginate bioink. The metabolic activity of the cells was assessed using the Alamar Blue assay. The results indicate that the metabolic activity of the cells gradually increases with time, with a significant increase on day 7, compared to days 1, 3, and 5. The metabolic activity was significantly increased on day 7 compared to day 1 in both dECM scaffolds and the bioprinted constructs (FIGS. 5A & 6C respectively). To further evaluate the biological activity and cell proliferation, the Pico green ds-DNA quantification assay was performed. The results (FIG. 5B) agree well with the Alamar Blue assay results, with a three-fold increase in DNA content on day 7 compared to day 1, indicating increased cell proliferation. While no significant differences were observed in the DNA levels between day 1 and day 3, the values significantly start increasing from day 5. The cells might initially take some time to attach and grow before proliferation. The total protein quantification (FIG. 5C) evaluated using the BCA assay also corroborate with the metabolic activity and DNA quantification results. The total protein content increased at all time points evaluated and there was a two-fold increase between day 1 and day 7.

SEM images of the cells seeded on tunics at different time points are shown in FIGS. 5D-F. FIG. 5D taken on day 1 shows cells being attached to the scaffolds. On day 5 (FIG. 5E), the cells can be clearly seen to be growing, migrating, and proliferating, forming layers of cell sheets, interconnected to each other. The morphology of the cells corresponds to the typical fibroblast cell morphology. The presence of filopodia seen on the leading edge of the cells are an indication that the cells are migrating within the tunic scaffolds. On day 7 (FIG. 5F), cells have completely covered the scaffold and three-dimensional network of cells through the ridges and grooves of the natural cellulosic substrate can be seen. Live/dead assay of the cells seeded on dECM scaffolds on day 1, 3, and 7, and bioprinted constructs on day 1 and 7, are shown in FIGS. 5G-I, and 6E-H respectively. DAPI-stained images of the bioprinted tissue constructs are shown in FIGS. 6F and 6I. A visibly large number of cells can be seen on the bioprinted constructs than on the dECM scaffold. This can be attributed to the more homogenous distribution of cells inside the bioink and better control over the cell density in the bioprinting process. Overwhelming majority of live cells with very few dead cells can be seen at all time points and increased cell numbers indicate the colony formation with the passage of time in both dECM and bioprinted constructs. Specifically, the live/dead image of the bioprinted construct on day 7 (FIG. 61) indicate a tissue-like formation.

Lyophilized dECM-Tunics as Wound-Dressing Materials Outperform the Commercial Dressing Materials with a Capacity of Absorbing 20 Times its Weight in the Dry State

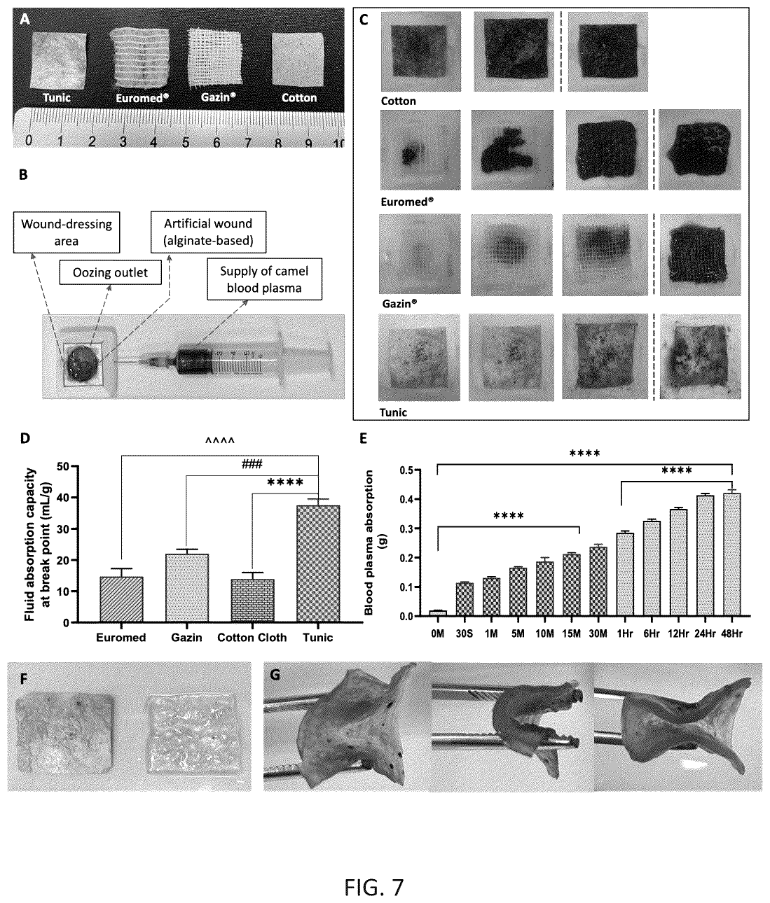

Given the high biocompatibility and fluid-absorption capacity of the tunics, the application of lyophilized tunics as a wound-dressing material was studied. The exudate absorption capacity of the lyophilized tunic is compared with the other commercially available wound-dressing materials such as cotton cloth, Euromed® and Gazin® (FIG. 7A). An artificial exuding wound is created using alginate-based hydrogel and camel blood plasma (FIG. 7B). The lyophilized tunic outperformed the other three commercially available wound-dressing materials in terms of the fluid absorption capacity at break point (FIG. 7D). While the cotton cloth and Euromed® absorbed around 11-16.5 mL/g before the exudate started leaking out of the dressing, Gazin® performed better with a value of 21-23.5 mL/g. The lyophilized tunic had the highest exudate absorption of 36-40 mL/g. FIG. 7C shows the different dressing materials before and after their break points. It can be clearly seen that the lyophilized tunic possesses high exudate absorption capacity with minimal leakage over a long period of time. There is significant absorption of blood plasma by the lyophilized tunics up until 24 hours (FIG. 7E), thus reducing the frequency of change of dressing. Images of the lyophilized tunic before incubation and after 48-hr incubation in camel blood plasma is shown in FIG. 7F. Another advantage of the lyophilized tunic over other wound-dressing materials is that the lyophilized tunic becomes a hydrogel-like construct and very flexible after exudate absorption (FIG. 7G), facilitating easy removal. The fibrous structures in other dressing materials might hurt the patient during dressing removal pulling part of the wound while the soft jelly-like nature of the tunic soothes the wound-area, giving a moderately moist environment, preventing the wound from drying. It has been proved by several clinical studies that a moist environment facilitates the wound-healing process by preventing dehydration, enhancing angiogenesis and collagen synthesis, increasing the breakdown of dead tissue and fibrin, and also decreasing the pain (Korting, H., Schöllmann, C., & White, R. (2011). Journal of the European Academy of Dermatology and Venereology, 25 (2), 130-137). Also, there are several other advantages of a moist wound-dressing including reduced re-epithelialization time, reduced inflammation and necrosis, minimized scar formation, and aid introduction of soluble agents such as growth factors, anti-microbial agents or other bioactive molecules (Junker et al. (2013). Advances in wound care, 2 (7), 348-356).

### Conclusion

In this work, environmentally destructive colonizing tunicate species of Polyclinum constellatum was identified in the coast of Abu Dhabi and methods to sustainably utilize the hazardous species as a valuable marine biomaterial were proposed. While tunicate-derived cellulose and nano-cellulose has been explored as a potential polymeric material before, the yield is only 5%, leaving the rest as waste. The native tunicate extra-cellular matrix, after decellularization and lyophilization, were used in three different ways: (i) as bioactive dECM tissue engineering scaffold (ii) wound-dressing material and (iii) formulation of bioink for regenerative medicine applications using 3D bioprinting. The intricate 3D nanofibrous cellulosic networks that remain intact even after the multi-step process of decellularization and lyophilization. The fact that the lyophilized tunics are dry and can be easily transported compared to other 3D culture systems such as hydrogels and on rewetting with PBS or culture media, the 3D tunic structure is regained, is a huge advantage for labs around the world trying to establish sustainable 3D culture systems. The tunic showed excellent biocompatibility, high mechanical properties (a modulus of 3.85±0.93 MPa compared to ˜0.1-1 MPa of hydrogels) and exhibited high fluid-absorption capability. Bioprinting MEFs suspended in tunic dECM-alginate bioinks proved the suitability of dECM tunics for bioink development with excellent post-printing cell viability and tissue-like formations. Experiments with camel blood plasma as wound exudate proved the superiority of the tunic over the other commercially available wound-dressing materials, with a capacity of absorbing 20 times its weight in the dry state. Since the lyophilized tunic becomes a gel after exudate absorption, it facilitates easy removal without causing pain and discomfort to the wound site. The jelly-like nature of the tunic helps maintaining a moist environment, conducive for wound-healing. Sustainable utilization of the ecologically-destructive tunicates species for biomaterials development can boost cleaner production and help significantly to control the ecologically destructive species.

### Example 2: A Designer Bioink to 3D Bioprint Human Neural Tissues for Translational Medicine Applications

Bioprinting of nervous tissue is a major challenge in tissue bioprinting field due to its soft consistency and complex architecture. The first step in efficient neural bioprinting is the design and optimization of printable bioinks which favor the growth and differentiation of neural tissues by providing the mechano-physiological properties of native tissue microenvironment. However, to date, limited studies have been conducted to make tissue specific bioinks. Here, disclosed is a novel bioink formulation specifically designed for bioprinting and differentiation of neural stem cells to peripheral neurons, using a marine tunicate-derived hydrogel. The formulation resulted in seamless bioprinting of neural stem cells with minimal processing time from bioink preparation to in vitro culture. The tissues exhibited excellent post-printing viability and cell proliferation along with a precise peripheral nerve morphology upon in vitro differentiation. The cultured tissues showed significant cell recovery after subjecting to a freeze-thaw cycle of −80° C. to 37° C. indicating the suitability of the method for developing tissues compatible for long term storage and transportation for clinical use. In conclusion, the study provides a robust method to use a sustainable bioink for 3D bioprinting of neural tissues for translational medicine applications.

Our understanding of nervous system disorders and its therapeutic developments majorly depends on the animal models and 2D cell culture systems. Most of these traditional models cannot address the questions that pertain to species variations, sensitivity, and complexity of the human nervous system. These limitations demand a more realistic in vitro human model to study the nervous system. Biomaterial engineering, 3D biofabrication, and stem cell technology can help design innovative tissue systems that can be used to model human nervous system physiology and patho-biology. Peripheral nervous system is a complex network of elongated nerves running throughout the body. Injury to peripheral nerves is a very common neurological disease that is generally caused by direct mechanical trauma or degeneration. The self-repairing ability of peripheral nerves is very limited and nerve injury can lead to life-long disability in affected persons. 3D Bioprinting of peripheral nerves is a promising technology to engineer peripheral nerve tissues for treatment as well as disease modelling. Stem cell technology combined with bioprinting offer important tools to make living peripheral nerve conduits and nerve tissues [Qiu, B., et al., Bioprinting Neural Systems to Model Central Nervous System Diseases. Adv Funct Mater, 2020. 30 (44): p. 1910250; Soman, S. S. and S. Vijayavenkataraman, Perspectives on 3D Bioprinting of Peripheral Nerve Conduits. Int J Mol Sci, 2020. 21 (16); Yu, X., T. Zhang, and Y. Li, 3D Printing and Bioprinting Nerve Conduits for Neural Tissue Engineering. Polymers (Basel), 2020. 12 (8)].

3D bioprinting requires the use of biocompatible bioinks, optimized for each cell types to favor the differentiation and growth of specific cell types for the formation of target tissues. The properties of the bioink determines the printability and how it integrates to form an extra cellular matrix around the cells to form the tissue-like structure. An ideal bioink should be biocompatible to the cell of choice, allow effortless printing, allow the miscibility of growth factors and media, help combat the shear stress of printing, should be compatible to crosslinking agents, and should promote the cell proliferation and differentiation [Ouyang, L., et al., Expanding and optimizing 3D bioprinting capabilities using complementary network bioinks. Sci Adv, 2020. 6 (38); Ouyang, L., et al., 3D Printing of Shear-Thinning Hyaluronic Acid Hydrogels with Secondary Cross-Linking. ACS Biomater Sci Eng, 2016. 2 (10): p. 1743-1751]. The viscoelastic properties of the bioink can be tuned for printing specific tissue types as well as to support specific cell populations [Gao, F., et al., Osteochondral Regeneration with 3D-Printed Biodegradable High-Strength Supramolecular Polymer Reinforced-Gelatin Hydrogel Scaffolds. Adv Sci (Weinh), 2019. 6 (15): p. 1900867]. Induced pluripotent stem cells (iPSCs) and iPSC derived stem cells are important materials for tissue bioprinting, as these cells can be differentiated to cells of choice when cultured in specific media. The printed tissue can be used for regenerative medicine application to make tissue transplants such as peripheral nerve conduits, brain patches and for neurodegenerative disease modelling [Soman, S. S. and S. Vijayavenkataraman, Applications of 3D Bioprinted-Induced Pluripotent Stem Cells in Healthcare. Int J Bioprint, 2020. 6 (4): p. 280]. Specific genetic line iPSCs, derived from patients, are a powerful tool to study diseases such as Parkinson's disease, Alzheimer's disease and cancer modelling. In the 3D bioprinting field, it has been presumed that the soft tissues such as brain and nerves require much optimization, as they are difficult to bioprint, compared to the hard tissues due to the finer variations in the viscoelastic properties of the hydrogels. Many recent research papers have reported the necessary conditions for 3D bioprinting neural tissues using soft hydrogel-based bioinks [Srubar, W. V., 3rd, Engineered Living Materials: Taxonomies and Emerging Trends. Trends Biotechnol, 2021. 39 (6): p. 574-583]. Researchers successfully bioprinted brain-mimicking tissues using primary cortical neurons mixed in a gellan gum-based bioink modified with the RGD peptide [Lozano, R., et al., 3D printing of layered brain-like structures using peptide modified gellan gum substrates. Biomaterials, 2015. 67: p. 264-73]. A recent work attempted to bioprint a model spinal cord using human iPSC-derived neural stem cells using an alginate-based bioink [Joung, D., et al., 3D Printed Stem-Cell Derived Neural Progenitors Generate Spinal Cord Scaffolds. Adv Funct Mater, 2018. 28 (39)]. However, most of these studies brought up the difficulty in proliferation of neural stem cells in traditionally used hydrogels [Madhusudanan, P., G. Raju, and S. Shankarappa, Hydrogel systems and their role in neural tissue engineering. J R Soc Interface, 2020. 17 (162): p. 20190505].

The advent of 3D bioprinting and tissue engineering has opened up a new discipline to precisely develop living human organ systems in vitro. Essentially, 3D bioprinting helps to biofabricate compatible biomaterials into desirable shapes designed with a software. Most of the bioprinted neural tissues have been generated using extrusion-based methods, laser-assisted printing, inkjet printing, drop-on-demand method, microfluidic printing technology and point-dispensing printing method [Bsoul, A., et al., Design, microfabrication, and characterization of a moulded PDMS/SU-8 inkjet dispenser for a Lab-on-a-Printer platform technology with disposable microfluidic chip. Lab Chip, 2016. 16 (17): p. 3351-61; Park, S., et al., Nanopatterned Scaffolds for Neural Tissue Engineering and Regenerative Medicine. Adv Exp Med Biol, 2018. 1078: p. 421-443; Shaqour, B., et al., Coupling Additive Manufacturing with Hot Melt Extrusion Technologies to Validate a Ventilator-Associated Pneumonia Mouse Model. Pharmaceutics, 2021. 13 (6)]. The most common method used for bioprinting neural tissue is extrusion bioprinting. In this type of bioprinting, one or more types of neural cells were mixed and suspended in a compatible hydrogel, and extruded in a layer-by-layer fashion according to a digital design, assisted by pressure, to form a tissue construct [Ouyang, L., et al., 3D Printing of Shear-Thinning Hyaluronic Acid Hydrogels with Secondary Cross-Linking. ACS Biomater Sci Eng, 2016. 2 (10): p. 1743-1751; Levato, R., et al., From Shape to Function: The Next Step in Bioprinting. Adv Mater, 2020. 32 (12): p. e1906423; Moroni, L., et al., Biofabrication strategies for 3D in vitro models and regenerative medicine. Nat Rev Mater, 2018. 3 (5): p. 21-37]. The choice of cells, the formulation of cell-specific bioinks and optimized printing parameters are the most important topics in bioprinting [Assuncao-Silva, R. C., et al., Hydrogels and Cell Based Therapies in Spinal Cord Injury Regeneration. Stem Cells Int, 2015. 2015: p. 948040]. It is considered difficult to optimize printing conditions for the soft tissues, because of their mechano-sensitive nature [Lozano, R., et al., 3D printing of layered brain-like structures using peptide modified gellan gum substrates. Biomaterials, 2015. 67: p. 264-73]. Compared to other types of cells, stem cells are more sensitive to sheer stress generated by the bioprinting process [Stolberg, S. and K. E. McCloskey, Can shear stress direct stem cell fate? Biotechnol Prog, 2009. 25 (1): p. 10-9]. So, it is essential to formulate bioinks and optimize printing methods that can protect the cells from the sheer stress and provide an ideal tissue microenvironment for the cell growth and cell differentiation to the intended cell lineage [Li, C., et al., Advances in the Fabrication of Biomaterials for Gradient Tissue Engineering. Trends Biotechnol, 2021. 39 (2): p. 150-164]. In case of peripheral neurons, the bioink should allow outgrowth of neurites and axons through within the printed construct [De Santis, M. M., et al., Extracellular-Matrix-Reinforced Bioinks for 3D Bioprinting Human Tissue. Adv Mater, 2021. 33 (3): p. e2005476; Echeverria Molina, M. I., K. G. Malollari, and K. Komvopoulos, Design Challenges in Polymeric Scaffolds for Tissue Engineering. Front Bioeng Biotechnol, 2021. 9: p. 617141]. An ideal bioink provides smooth flow through the nozzles without any clogging that will reduce the total printing time and cellular stress.

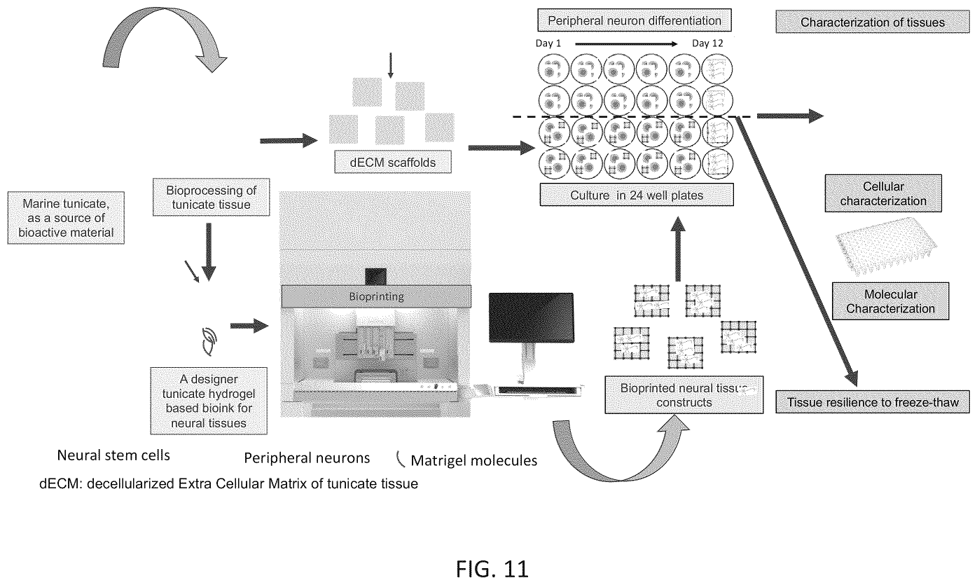

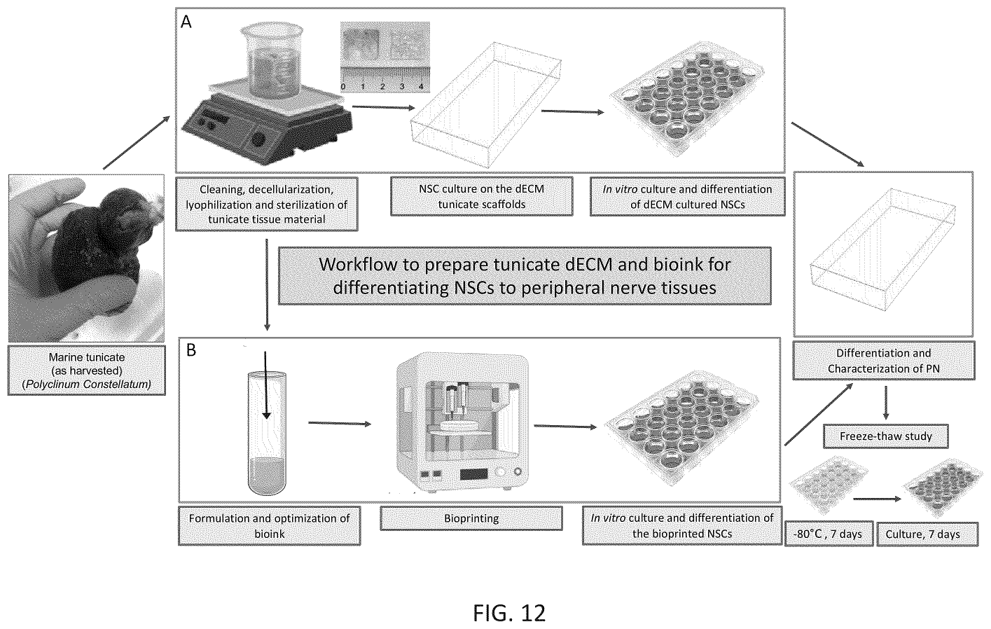

In this work, disclosed is a novel marine tunicate-based bioink to 3D bioprint neural stem cells and its differentiation into peripheral neurons (PN). The cytocompatibility of the marine tunicate dECM scaffolds was evaluated by culturing and differentiation of the human iPSC derived Neural Stem Cells (NSCs) into peripheral neurons (PN) (FIG. 11). Further, a bioink using the tunicate dECM powder and Matrigel was formulated and optimized for bioprinting of NSCs that differentiated in vitro into peripheral neurons (FIG. 11). The bioink formulation and bioprinting parameters were optimized for bioprinting NSCs that proved to be efficient in providing a conducive tissue microenvironment for the PN differentiation. Lattice-shaped neural tissue constructs were bioprinted in a dish and their cellular properties and cold resistance potential were characterized. The neural tissue cultures and constructs were analyzed for cell viability, cell proliferation and cell differentiation as peripheral neurons (FIG. 12). The current work, expands the scope of bioprinting by adopting a novel sustainable bioink for neural stem cells and its differentiation in lab for regenerative medicine applications and disease modelling.

### Materials and Methods

### Cell Culture

iPSC-derived normal human Neural Stem Cells (NSCs) were purchased from AddexBio, San Diego, USA (Catalogue number P0005048). The cell culture plates were coated with Matrigel and 1×10⁶ cells were seeded onto one well of a 6 well plate. The cells attached on the plates in 24-48 hours. NSCs were cultured in 5% CO₂ at 37° C. with alternate day media changes using Neural Stem Cell Growth Medium (Catalogue number C0013-09, AddexBio). The NSC cultures were scaled up for seeding onto the tunicate dECM scaffolds and for bioprinting.

Decellularization of Tunicate Extra Cellular Matrix (dECM) Scaffold

Fresh tunicates (Polyclinum constellatum, NCBI Accession number MW990087) were collected from the Zayed Port, Abu Dhabi, United Arab Emirates. The samples were thoroughly washed with deionized water. The outer rough layer of the tunicates was removed using a sterile surgical knife and the whole hydrogel-like tunic tissue was separated into a culture dish. The tunicate tissue was cleaned using deionized water at room temperature few times, before being cut into required dimensions. The tunic tissue pieces are stirred in decellularization buffer consist of 10 mM Tris, 1 mM of ethylenediamine tetra acetic acid (EDTA), 0.2% V/V of Triton X-100, and 1.5% of sodium dodecyl sulfate, at a pH of 7.5 for 48 hours. The buffer was changed every 2 hours until 10 hours. Cellular debris were removed by washing with deionized water after 10 hours. The decellularized tissue pieces were cut into dimensions of 1 cm×1 cm×˜0.1 cm for NSC seeding for cytocompatibility, proliferation and differentiation studies.

Culture and Differentiation of NSCs on Tunicate dECM Scaffolds

The scaffolds were sterilized for one hour in UV irradiation in a biosafety cabinet. The sterilized scaffolds were washed three times with prewarmed PBS and one time with the NSC medium. Confluent cultures of NSCs were harvested using accutase enzyme and washed in the NSC media. The cells were counted and concentrated to 3×10⁶ cells in 30 μL volume of NSC medium and seeded on to the dECM scaffolds placed in the wells of 24 well plates. The plates were incubated in a 5% CO₂ incubator at 37° C. After 4 hours of incubation, 0.5 mL of NSC media was added. The culture media was changed after 24 hours and then every alternate day. After three days of culture, the NSC medium was replaced with the PN induction medium. The PN media was composed of neurobasal media (Thermo fisher scientific) supplemented with 1× non-essential amino acids, 1× GlutaMAX™ (Sigma), 1×N2, 1×B27 (Thermo fisher scientific), 20 ng/ml EGF (Sigma), 20 ng/ml bFGF, 10 ng/ml nerve growth factor-β, and 25 μM Y27632 (Merck Millipore) for differentiation of NSCs to PN. Media changes were performed once every three days for two weeks [Zhu, Q., et al., Directed Differentiation of Human Embryonic Stem Cells to Neural Crest Stem Cells, Functional Peripheral Neurons, and Corneal Keratocytes. Biotechnol J, 2017. 12 (12)]. The cells were cultured in the PN induction medium for another 12 days and checked for the NSC to PN differentiation using specific markers at day 7 and day 12.

Cell Viability in the Tunicate dECM Scaffolds

The cell viability and proliferation in the dECM tunicate scaffolds were analyzed on day 3, 7 and 12. The dECM tunicate scaffolds with cells and without cells were stained with Calcein AM and Ethidium homodimer1 (Invitrogen LIVE/DEAD™ Viability/Cytotoxicity Kit, for Mammalian Cells, catalogue number L3224). Prior to staining, the cells were washed with prewarmed physiological saline. The cells in the dECM tunicate scaffolds were stained with 500 μL of 2M Calcein AM and 4M Ethidium homodimer1 working solution for 45 minutes at room temperature. After the incubation, the dECM tunicate scaffolds were lifted from the wells and mounted on a clean slide followed by confocal imaging using a Leica SP8 confocal laser scanning microscope. In this staining method, live cells are distinguished by the presence of ubiquitous intracellular esterase activity, determined by the enzymatic conversion of the virtually nonfluorescent cell-permeant Calcein AM to the intensely fluorescent Calcein. The polyanionic dye Calcein is well retained within live cells, producing an intense uniform fluorescence in live cells. Ethidium homodimer1 dye enters cells with damaged membranes and undergoes a 40-fold enhancement of fluorescence upon binding to nucleic acids, thereby producing a bright red fluorescence in dead cells.

Cell Proliferation on Tunicate dECM Scaffolds

Alamar blue assay (AlamarBlue HS Cell Viability Reagent, Invitrogen, Catalogue number A50101) was used as a measurement for the determination of cell viability and proliferation. Cell growth was analyzed at different time points: 3, 7 and 12 days. Scaffolds were incubated with 10 μl of Alamar blue solution per each 100 μL (1:10 ratio) of media and incubated for 4 hours. The Alamar blue reaction mix was collected in a 96 well plate and the absorbance was measured at a wavelength of 570 nm with a reference wavelength of 600 nm using a microplate reader (Epoch, BioTek). The percentage reduction of the Alamar blue reagent, which is linear measurement of the viable cells in the culture was calculated using the online AlamarBlue colorimetric calculator (Biorad).

mRNA Expression of PN Markers

The RNA from cultured cells were isolated using Qiaquick RNA extraction kit (Qiagen) according to the manufacturer's instructions. The extracted RNA was quantified using Nanodrop ND-1000 spectrophotometer (Nanodrop technologies, Wilmington, DE). 1 μg of mRNA was reverse transcribed into cDNA using Superscript Vilo IV cDNA Synthesis Kit (Thermo Fisher Scientific). Real-time quantitative PCR reactions were carried out in triplicates with 500 ng cDNA template per reaction using SYBR master mix (Thermo Fisher Scientific) in a Step oneplex Real-Time PCR System (Applied Biosystems). mRNA of Neural markers; TUBB3 (β3 tubulin), a pan-neuronal marker, Peripheral neuron specific markers Peripherin (PRPH), Neurofilament heavy polypeptide (NEFH) and a stemness marker HNKI were analyzed in the day 7 and day 12 induced samples. The sequences of the forward and reverse primers of genes; analyzed were adapted from Vijayavenkataraman et. al., 2019 [Vijayavenkataraman, S., et al., 3D-Printed PCL/PPy Conductive Scaffolds as Three-Dimensional Porous Nerve Guide Conduits (NGCs) for Peripheral Nerve Injury Repair. Front Bioeng Biotechnol, 2019. 7: p. 266]. The target gene expression was normalized to the house keeping gene GAPDH. The results were expressed as relative mRNA expression compared to the day 3 samples.

### Immunocytochemistry of PN Protein Marker

The NSC seeded scaffolds or neural tissue constructs were fixed with 4% paraformaldehyde (Sigma) for 10 min at room temperature. Fixed cells were permeabilized using 0.1% TritonX-100 in PBS (Sigma) for 15 min, washed thrice with 0.05% Tween-20/PBS (Sigma), and blocked with 1% bovine serum albumin for 1 hour to avoid non-specific binding. Subsequently, the cells were incubated with Rabbit Anti-Neurofilament heavy polypeptide (NEFH) antibody (1:50 in 1% BSA in PBS, ab8135, Abcam) at room temperature for 1 hour. The scaffolds or constructs were washed with PBS for three times and incubated with fluorescent labelled secondary antibody Goat Anti-Rabbit IgG H&L (Alexa Fluor® 488, ab150077, Abcam) for 1 hour at room temperature, then counterstained with 1 μg/mL of nuclear stain, DAPI (4′,6-diamidino-2-phenylindole, Sigma). The images were taken with a Leica SP8 confocal laser scanning microscope and analyzed.

### Scanning Electron Microscopy (SEM)

After day 3 of culturing of NSCs on the tunicate dECM scaffolds, the cell-loaded scaffolds appeared impermeable to light, hence SEM was carried out to get a clear picture of the cell growth and differentiation. For SEM, the cultured scaffolds were fixed in 4% paraformaldehyde for 1 hour at room temperature and dehydrated with serial concentration of ethanol ranging from 50%, 70% and 100%, then freezed in critical point dryer. The scaffolds were then coated with gold and imaged in a Scanning Electron Microscope (Qanta, Thermo Fisher Scientific).

### Design of Tissue Constructs

Scaffolds were designed and fabricated using RegenHU 3D Discovery printer BioCAD software (RegenHU, Switzerland). They were designed in a square 8 mm×8 mm grid pattern with a line spacing of 2 mm and a total thickness of 0.5 mm, with each layer being 0.5 mm thick comprising vertical and horizontal struts. Using a built-in software well plate editor the toolpath was calibrated to print the constructs in a 24 well plate format. The tool path was generated and saved as an iso file in the BioCAD software.

### Bioink Preparation

For making the bioink, the decellularized tissue pieces were frozen at −80° C. overnight and lyophilized using a Christ Alpha 1-2 LD Lyophilizer for 48 hours. The lyophilized scaffolds were sterilized with ethanol and UV radiation before making the tunicate powder. For making the powder, the dECM tissues were sliced into smaller sections and immersed in liquid nitrogen (˜5 mL) in a mortar. The frozen tissue sections were pulverized into fine powder using a mortar and a pestle. After lyophilization, the ECM powder was mixed with pepsin enzyme in a ratio of 10:1 w/w per 100 mL 0.01N HCl. The solution was digested for 48 hours at room temperature under constant stirring using a magnetic stir bar and plate until the solution becomes viscous with no visibly undigested granules. Then, 10 mg/mL of digested ECM solution was aliquoted and frozen at −80° C. to terminate pepsin digestion. Further, the digested ECM solution was mixed well and dialyzed against water at 4° C. for 72 hours. Finally, the obtained ECM powder was freeze-dried and lyophilized for further use. All the buffer components and chemicals used for ECM powder preparation were from Sigma-Aldrich, USA. The tunicate powder was sterilized using UV irradiation for 2 hours before preparing the base hydrogel. The base hydrogel for bioprinting was prepared by slowly adding NSC media to 100 mg of tunicate powder to make a final volume of 1 mL. The hydrogel concentration was optimized for NSC bioprinting by adding different concentrations of Matrigel (Matrigel hESC-qualified Matrix, Catalogue number 354277, Corning) starting from 50%, 35%, 31% and 26%. The higher concentrations of Matrigel made the hydrogel more viscous and did not allow printing. 26% of the Matrigel in the base hydrogel was found to be optimal and facilitated smooth printing of the cell-free scaffolds. Therefore, this formulation was used to make the bioink for cell printing. A bioink containing 26% Matrigel, 10% tunicate powder and 0.1 mL of the NSC suspension containing 7.58×10⁶ cells was formulated (a total volume of 1 mL), which could print 20 tissue constructs in a 24 well plate. Each of the constructs consumed ˜50 μL of the bioink, with ˜4×105 NSCs. Matrigel was kept at 4° C. before being added to the media. The preparation of bioinks was carried out in a biosafety cabinet at room temperature within 15 minutes before printing.

### 3D Bioprinting

RegenHU 3D discovery bioprinter inside a biosafety cabinet at room temperature was used for bioprinting. The printer and the biosafety cabinets were sterilized under UV light for one hour prior to printing. Bioink containing NSCs were loaded in a 3 mL sterile syringe and connected to the air pressure supply. A needle with 0.51 mm inner diameter was used for the printing (Needle DD-135N ID-0.51/G21 L=25.4, RegenHU, Switzerland). Print parameters were adjusted to obtain continuous flow rate and smooth hydrogel fibers with minimal spreading. A feed rate of 2 mm/s and pressure of 0.3-0.4 MPa were used, the total print time was under 30 minutes per one 24 well plate. The printability of the bioink was assessed by switching on the pressure and the filament formation at the tip of the needle. The needle diameter, pneumatic pressure and nozzle moving speed were optimized to deliver continuous extrusion of the bioink in the designated well of the well plate.

### Crosslinking of the Printed Tissue Constructs and Tissue Culture

For optimization of printing and crosslinking, the cell-free control hydrogel filaments were immersed in a crosslinking solution and PBS to check the strength of the filament formation. Immersion in 250 mM of CaCl₂) could make a smooth filament of crosslinked hydrogel at room temperature. The blue stain Alcian blue is added to the control hydrogels for better visibility. The same concentration of the CaCl₂) solution was used to crosslink the cell containing tissue constructs after bioprinting. The crosslinker solution was removed after 5-10 min of incubation at room temperature and the constructs were washed with prewarmed PBS. After washing with PBS, the cell-laden constructs were incubated in nutrient-rich NSC media containing 10% FBS at 37° C. in 5% CO₂. The nutrient rich media was changed to NSC medium after 15 minutes.

### Peripheral Neuron Differentiation of Bioprinted Tissue Constructs

Bioprinted constructs were initially cultured in the NSC media for 5 days; once the cells adapted to the new 3D environment, they were induced with PN media for differentiation to PN. The tissue constructs were analyzed for cell viability and cell proliferation as described for the dECM scaffolds. The tissue constructs were analyzed for PN-specific marker expression by immunofluorescence and qPCR with the same procedure used for the dECM scaffolds. SEM was done to see the tissue construct morphology after culturing and differentiation.

Viability and Proliferation of the Freeze-Thawed dECM-Grown and Bioprinted PN

On day 12 of induction, the differentiated PN grown on the dECM scaffolds and bioprinted tissues were washed twice using the fresh prewarmed PN media. Then the tissues were immersed in 1:1 ratio of PN media and cell freezing media (Embryomax freezing media, Catalogue number ES002D, EMD Millipore), incubated at room temperature for 10 minutes. The 24 well plates containing tissues were frozen directly at −80° C. After one week of freezing, the plates were taken out and kept at 37° C. for 15 minutes. The thawed freezing media was removed and the tissues were washed twice in 0.5 mL of prewarmed fresh PN media. The tissues were further cultured in the PN media for a week with alternate day media changes. The cell viability and proliferation were assessed by Alamar blue assay and live-dead staining.

### Statistical Analysis

Results were analyzed statistically. All graphical data represent the mean+/−standard deviation of at least three independent experiments. Differences between treatments were tested using the two-tailed student's t test. * p<0.05, ** p<0.01, *** p<0.001, **** p<0.0001, ***** p<0.00001 were considered statistically significant in all cases.

### Results and Discussion

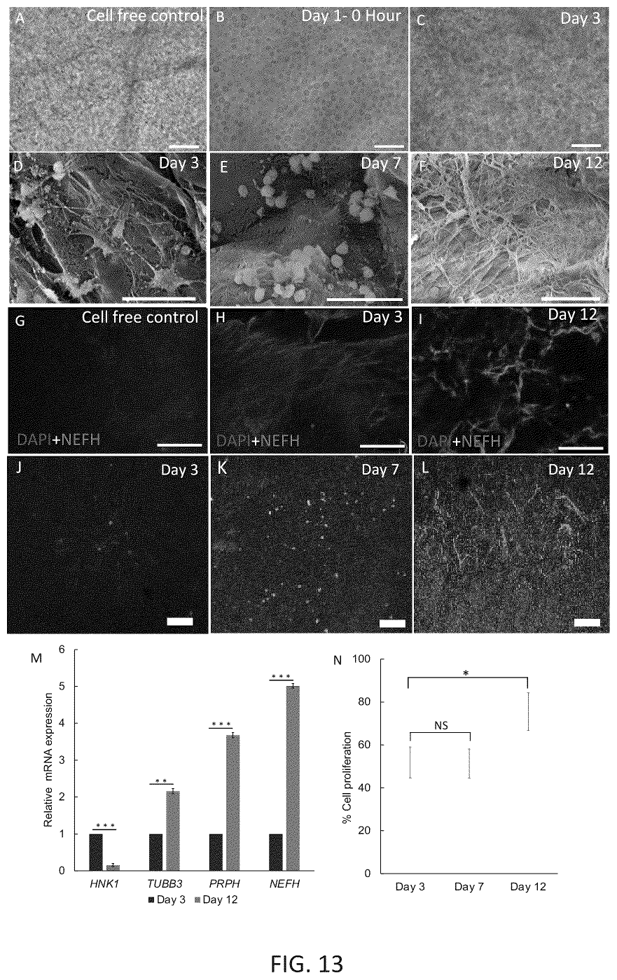

To design a cell-specific bioink for neural tissues, the key factors considered in this study were the type of cells and the choice of biomaterials used. The tunicate tissue majorly composed of biocompatible and biodegradable cellulose [Athukoralalage, S. S., et al., 3D Bioprinted Nanocellulose-Based Hydrogels for Tissue Engineering Applications: A Brief Review. Polymers (Basel), 2019. 11 (5)]. The decellularization process of the tunicate tissue could yield clean, transparent looking scaffolds with natural pores, and it was examined whether they would aid in the cell adherence and proliferation. Since the stem cells are sensitive to many intrinsic and extrinsic factors for growth such as coating matrix and cell seeding density, the cytocompatibility of these scaffolds were tested using mouse embryo fibroblasts (MEFs) in a recently published work [Govindharaj, M., et al., Bioprinting of bioactive tissue scaffolds from ecologically-destructive fouling tunicates. Journal of Cleaner Production, 2022. 330]. The scaffolds offered good cytocompatibility and growth of MEFs. Following the initial success with MEFs, in this work, NSCs were seeded on the scaffolds without any matrix coating (usually neural stem cells are seeded on a Matrigel-coated surface) at a high seeding density and neural induction was given on day 3 of the culture. The NSCs were attached to the dECM tunicate dECM scaffolds by 24-48 hours after seeding and started the colony formation. The scaffolds became thick and impermeable to light by day 3 (FIGS. 13A, 13B, and 13C). So, further imaging was carried out using scanning electron microscopy (SEM). On day 3 of PN induction (FIG. 13D), the electron micrograph of the NSCs on the scaffolds still showed the fibroblast-like morphology, which changed to a more rounded cell appearance by day 7 (FIG. 13E) with distinct colony formation, filling in the natural pores of the scaffolds. By day 12 of neuronal induction, the cells showed clear morphology of peripheral neuronal fibers, which formed a network of PN with elongated neural outgrowth throughout the surface of the scaffolds (FIG. 13F). It was examined whether these morphology changes might be due to the mechanical surface cues the cells experience from the rough surface of the scaffold.

Cell viability of NSCs seeded on tunicate dECM scaffolds was analyzed on days 3, 7 and 12 of culture. Live-dead staining was used to see the viable cells on scaffolds and colorimetric Alamar blue assay was used for the quantification of cell proliferation. The live-dead assay of cells grown on the tunicate dECM scaffolds showed an increase in the number of green florescent cells over time and proliferation of thread-like structures by day 12 (FIGS. 13J, 13K, and 13L). Alamar blue assay showed significant cell proliferation on day 12 compared to day 3 (FIG. 13N). Between day 3 and day 7, the proliferation was not significant, probably because the cells are adapting to the new culture environment along with the pressure of cellular differentiation to PN (FIG. 13N) [Altman, G. H., et al., Cell differentiation by mechanical stress. FASEB J, 2002. 16 (2): p. 270-2]. These experiments prove that the tunicate dECM scaffolds provide a very conducive tissue microenvironment for the growth of different types of cells, including neural stem cells.

The immunofluorescence staining of the cells in different stages of the induction showed expression of peripheral neuron marker Neurofilament heavy polypeptide (NEFH) by day 12, which indicate the differentiation of the NSCs to PN on the scaffolds with the induction media. The non-cell loaded and day 3 controls did not show NEFH expression (FIGS. 13G, 13H, and 13I). The quantitative polymerase chain reaction (qPCR) was performed to analyze the mRNA expression of the PN markers on day 3 and day 12 of PN induction. The relative mRNA expression of NEFH and PRPH expression increased four to five-fold on day 12 of the PN induction, compared to the day 3 samples, while the HNKI gene expression was remarkably reduced (FIG. 2M). HNKI is a stem cell marker, the reduction of this gene indicates the differentiation of stem cells to peripheral neurons. Day 12 neurons also showed slightly increased levels of the pan neuronal marker TUBB3 (FIG. 13M). The gene expression was normalized to house-keeping gene GAPDH (FIG. 13M).

The base hydrogel for bioprinting was optimized before adding the cell component. The tunicate hydrogel and different concentrations of Matrigel in the NSC media were used for making the base hydrogel. Matrigel is a matrix protein which polymerize at physiological temperature; therefore, aliquots of Matrigel were stored at 4° C. before mixing with NSC media. High concentrations of Matrigel (>30%) in the base hydrogel caused clogging of the needle and did not extrude from the nozzle due to increased viscosity and rapid solidification within the needle. Even when the printing pressure was increased from 0.45 MPa to 0.65 MPa, no extrusion happened. At a lower Matrigel concentration (26%), the hydrogel extruded smoothly and the lattices of cell-free scaffolds were printed in less than 1 minute/scaffold. NSCs were added to this formulation to make the bioink for neural tissue printing. The formulation followed the same seamless pattern as that of the cell-free printing.

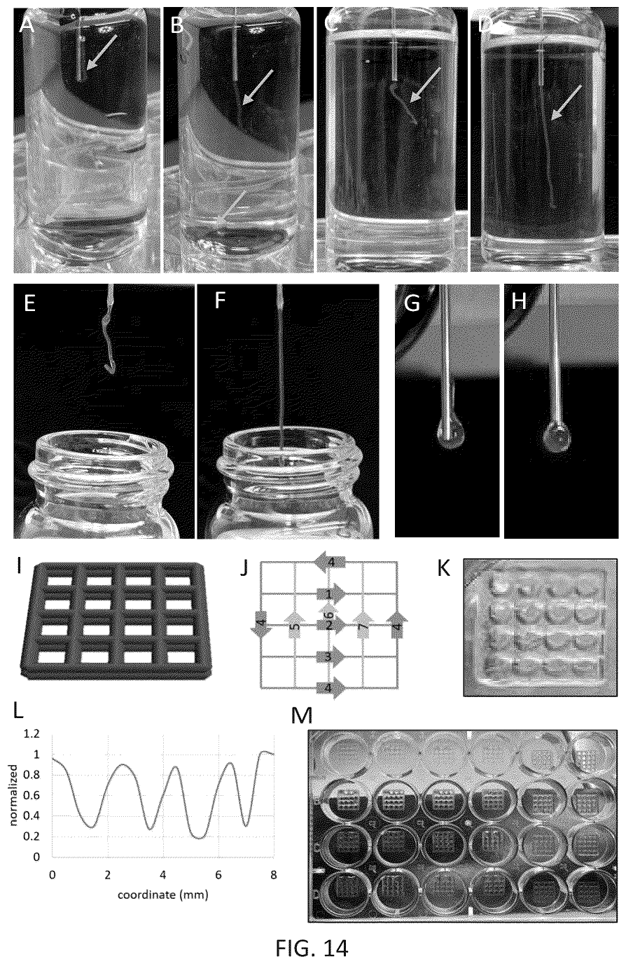

Crosslinking of the printed constructs was optimized using the combinations of cell-free hydrogel controls. Alcian blue was added to the hydrogel to give clear visibility in the liquid interphase of the crosslinking solution. The optimized formulation showed quick and stable crosslinking while treated with the ionic crosslinker CaCl₂). The filament formation of the hydrogel was consistent on printing within a 250 mM CaCl₂) solution when compared to the physiological saline (PBS) (FIG. 14A-14F). On addition of Matrigel, hydrogel with Matrigel showed consistent droplet formation and downward flow compared to the hydrogel without Matrigel (FIG. 14G, 14H).

For the bioprinting experiments, the tissue constructs were designed and fabricated using RegenHU 3D Discovery printer (FIG. 141). The tool path for the tissue construct was also generated (FIG. 14J). The design facilitated the deposition of the bioink in a layer-by-layer fashion with the dimensions of the tissue construct being 8 mm×8 mm×1 mm. The pore size of the lattice was measured as ˜900 μm using the digital camera images (FIG. 14K). The bioprinted lattice showed uniformity in the strut size and the pore size as measured in the bioprinted tissue constructs (FIG. 14L). This study used 0.51 mm diameter needle and the number of layers was set as 2, which gives an approximate height of the construct as 1 mm. The chosen dimensions facilitated the mounting of the tissues in a glass slide for imaging and for the scaling up of the tissue production using specific quantities of the bioink.

Cell-laden constructs were printed after adding the NSCs to the optimized hydrogel, crosslinked post-printing and cultured in vitro (FIG. 14K). The crosslinked tissue constructs stayed soft enough to allow the cellular activities such as adherence, migration, proliferation and differentiation and at the same time possessed enough post-printing structural stability and stiffness to form a nerve tissue throughout the in vitro culture period. The method was scaled-up to automate the printing process of printing tissue constructs in 24 well culture plates, expanding the scope of bioprinting to develop disease-in-dish models and for making human tissues for regenerative medicine applications (FIG. 14M).

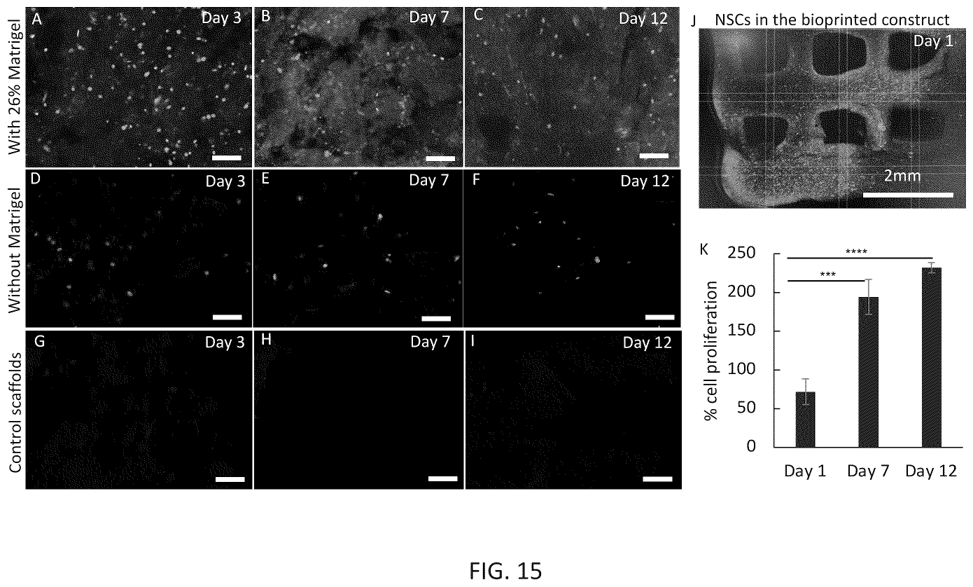

The neural growth and building of neural network from the stem cells in vitro require the guidance of axons in an efficient and long-lasting manner. The experiments proved that the formulated bioink provide ideal conditions for the 3D neural outgrowth [Qiu, B., et al., Bioprinting Neural Systems to Model Central Nervous System Diseases. Adv Funct Mater, 2020. 30 (44): p. 1910250]. Many different factors influence cell differentiation and axon protrusion in vitro. Here, modifications to the neural tissue environment using both physical and chemical stimuli are made. The physical environment was modified by optimizing the bioink composition by adding specific concentration of Matrigel. Initial experiments of NSC differentiation on dECM scaffolds (FIG. 13) did not require Matrigel coating for the cell growth and differentiation. But, when the bioinks were formulated without Matrigel for bioprinting, the NSCs did not grow well, contrary to the expectations (FIGS. 15D, 15E, and 15F). To provide a more cell-friendly environment, Matrigel was then added to the dECM bioink. Matrigel is a reconstituted basement membrane derived from extracts of Engelbreth-Holm-Swarm mouse tumors. The tumor basement membrane consists of a thin layer of ECM sheets that are primarily made up of type IV collagen, entactin, heparan sulfate proteoglycans, laminin, and growth factors to support cell growth [Duarte Campos, D. F., et al., Bioprinting Cell- and Spheroid-Laden Protein-Engineered Hydrogels as Tissue-on-Chip Platforms. Front Bioeng Biotechnol, 2020. 8: p. 374; Saldin, L. T., et al., Extracellular matrix hydrogels from decellularized tissues: Structure and function. Acta Biomater, 2017. 49: p. 1-15t]. Matrigel closely resembles the complex extracellular environment of the basement membrane, where cells adhere during tissue formation. Both the live-dead staining (FIGS. 15A, 15B, and 15C) and Alamar blue cell proliferation assay (FIG. 4K) showed highly significant cell growth by day 7 and day 12 post-printing in the Matrigel-containing bioink. From these results, it can be inferred that the addition of Matrigel to the tunicate dECM bioink aids better cell encapsulation and favor enhanced cell adhesion and growth [De Santis, M. M., et al., Extracellular-Matrix-Reinforced Bioinks for 3D Bioprinting Human Tissue. Adv Mater, 2021. 33 (3): p. e2005476]. While the dECM scaffold, without any matrix coating, favored cell adhesion, growth, and differentiation, it could be probably due to its inherent porous structure as the NSCs were directly seeded onto the scaffold, without subjecting the cells to any undue stress (as with bioprinting). It was examined whether the addition of Matrigel might have helped resist the cellular stress generated by the bioprinting procedure, which was evident from the better cell proliferation in the Matrigel-containing bioink. The bioprinted tissue showed more cell proliferation compared to the dECM cultured cells due to the presence of Matrigel and also due to better exchange of nutrients in all parts of the construct than the dECM scaffolds.

With 3D bioprinting, it is a difficult task to find the bioink formulations that is printable with good post-printing structural stability and at the same time provide the physicochemical cues to meet the biological needs of the cells for differentiation, as these characters of the bioinks are mutually exclusive with many hydrogels [Baena, J. M., et al., Volume-by-volume bioprinting of chondrocytes-alginate bioinks in high temperature thermoplastic scaffolds for cartilage regeneration. Exp Biol Med (Maywood), 2019. 244 (1): p. 13-21; Sharma, R., et al., 3D Bioprinting Pluripotent Stem Cell Derived Neural Tissues Using a Novel Fibrin Bioink Containing Drug Releasing Microspheres. Front Bioeng Biotechnol, 2020. 8: p. 57; Skylar-Scott, M. A., et al., Biomanufacturing of organ-specific tissues with high cellular density and embedded vascular channels. Sci Adv, 2019. 5 (9): p. eaaw2459]. Most of the high shape fidelity bioinks are highly viscous and pose difficulty in printing due to nozzle clogging. There were difficulties in extruding the exemplary bioink with a high percentage of Matrigel as a bioink component. Matrigel-containing bioink required more care and optimization as it contributed to the temperature and time sensitivity during printing. At room temperature, high percentage Matrigel (>30%) bioink solidified faster in the needle, resulting in clogged nozzles. This delayed the whole printing process and undesirable printing outcomes, as the cells experience more stress during the printing process. So, it is important to develop easy-to-use simple formulations which will work well in ambient temperatures without causing any printing delay [Bernal, P. N., et al., Volumetric Bioprinting of Complex Living-Tissue Constructs within Seconds. Adv Mater, 2019. 31 (42): p. e1904209]. The formulation of 10% tunicate dECM gel and 26% Matrigel showed consistent seamless printability of NSCs at room temperature. The preparation of bioink took ˜15 minutes (until loading the bioink cartridge onto the printer) and the printing of each scaffold took ˜1 minute. The total time required to print a 24 well plate was approximately ˜24 minutes, which is optimal for stem cell bioprinting.

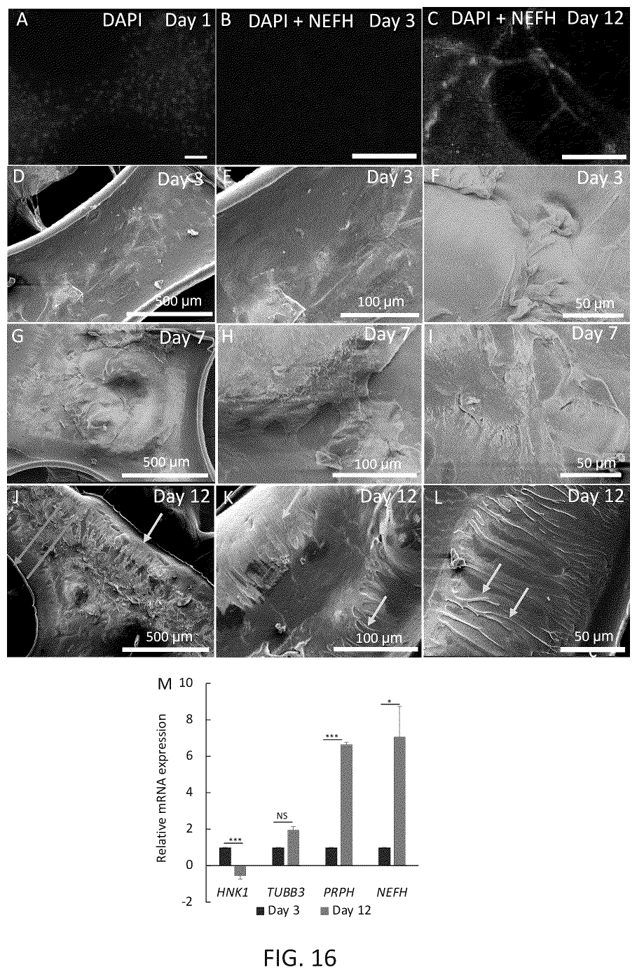

In the bioprinted tissue constructs, the NSCs started to proliferate by 3-5 days post printing. On day 5, once the cells appeared settled to grow, PN induction media was added. The immunofluorescence staining of the cells on different stages of the induction showed expression of peripheral neuron marker NEFH by day 12, indicating the differentiation of the NSCs to PN inside the tissue constructs (FIG. 16C), while the day 3 sample did not stain for NEFH (FIG. 16B). By day 12 of neuronal induction, the cells showed clear morphology of peripheral neuronal fibers, which formed a network of PN in the tissue constructs. The scanning electron micrograph of the PN cells showed typical PN morphology protruding on the surface of the tissue construct. The direction of the cells appeared perpendicular to the direction of the printing (FIG. 16L). This observation requires more detailed investigation in the future, to know the physiological determinants of the directionality of the nerve formation [Keshavarz, M., et al., Induced neural stem cell differentiation on a drawn fiber scaffold-toward peripheral nerve regeneration. Biomed Mater, 2020. 15 (5): p. 055011; Wang, J., et al., The spatial arrangement of cells in a 3D-printed biomimetic spinal cord promotes directional differentiation and repairs the motor function after spinal cord injury. Biofabrication, 2021. 13 (4); Wen, Z. and J. Q. Zheng, Directional guidance of nerve growth cones. Curr Opin Neurobiol, 2006. 16 (1): p. 52-8], which can possibly give valuable information for PN injury repair. The quantitative polymerase chain reaction was performed to analyze the mRNA expression of the PN markers on day 3 and day 12 of PN induction (FIG. 16M). The mRNA expression of PN markers; PRPH and NEFH were upregulated on day 12 of PN induction compared to day 3. The stemness marker HNKI was significantly downregulated and the change in the pan neural marker TUBB3 was non-significant (FIG. 16M). The mRNA profile (FIG. 16M) gives clear clues on the PN differentiation.

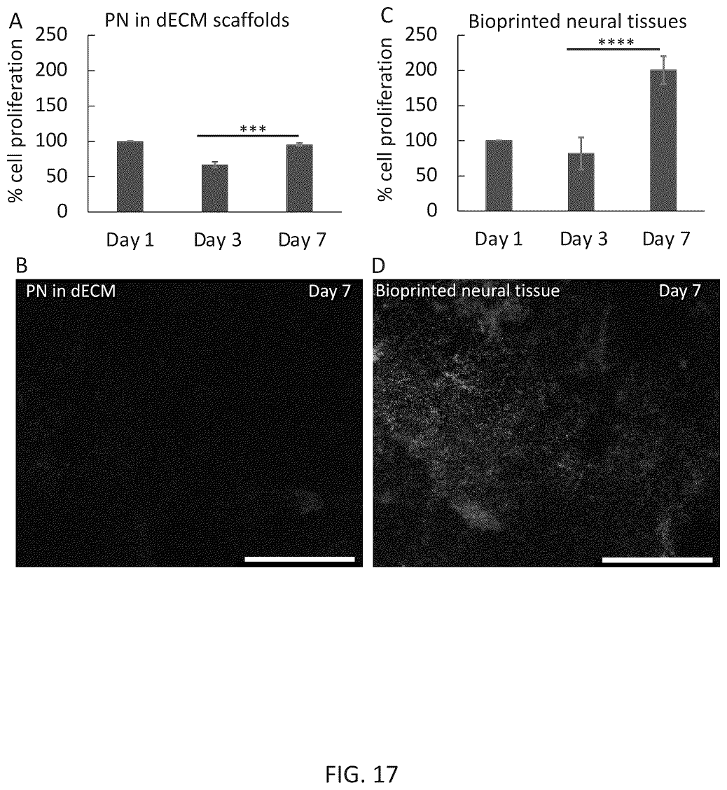

One of the ultimate aims of tissue bioprinting is future regenerative medicine applications. This requires short- or long-term storage of tissues and tissue transportation in ultra-low temperatures. A freeze-thaw study was conducted to analyze the storage potential of the bioprinted tissues (FIG. 17). The ability of the bioprinted and dECM scaffold tissues for its cold shock recovery was analyzed after keeping them frozen for a week in a freezing media at −80° C. The cold shock recovery of the tissues was assessed using cell viability and cell proliferation assays. The cells showed initial slow recovery after the cold shock, but recovered and proliferated on culturing them further for a week. The bioprinted tissues showed better cell proliferation compared to the dECM scaffold-tissue. The results are encouraging to explore further on the storage and transportation of bioprinted tissues for translational medicine applications [Murphy, S. V., P. De Coppi, and A. Atala, Opportunities and challenges of translational 3D bioprinting. Nat Biomed Eng, 2020. 4 (4): p. 370-380; Vijayavenkataraman, S., et al., 3D bioprinting of tissues and organs for regenerative medicine. Adv Drug Deliv Rev, 2018. 132: p. 296-332].

### Conclusions

In conclusion, the disclosed example demonstrated seamless bioprinting and differentiation of NSCs to PN using a custom-designed bioink for neural tissues. The example optimized the bioprinting workflow at room temperature, which makes it easy to handle and quick to print. The printed tissue constructs maintain the soft tissue consistency required for the nervous tissue throughout the culture period and exhibited high cell viability and proliferation. Upon induction, the bioink aided the formation of peripheral nerve tissues with well-formed neurites. The cultured tissues showed significant recovery from cold shock at −80° C., which is a promising observation to use this method to develop tissues for clinical use. Moreover, the disclosed bioprocessing method efficiently use an untapped source of biomaterial to design tissue specific bioinks. The development of sustainable bioinks from marine invasive tunicates would open up new avenues for scaling up the hydrogel-based soft tissue bioprinting for their application in translational medicine.

### Example 3: 3D Bioprinting and Post-Bioprinting Differentiation of Human Mesenchymal Stem Cells into Chondrogenic Tissues in a Novel Tunic dECM Bioink

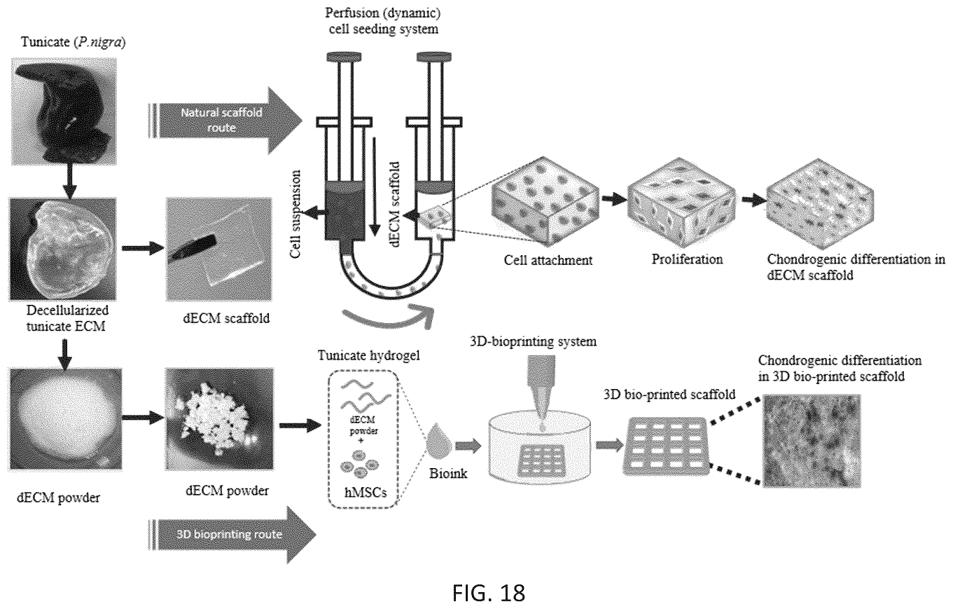

Tunicates are marine organisms renowned for their thick, leathery, organic exoskeleton called tunic. This tunic is composed of an extracellular matrix packed with protein-cellulose complexes and sulfated polysaccharides, making it a charming option as a biomaterial in cartilage tissue engineering. In this study, P. nigra tunicate was collected and decellularized to obtain its rich decellularized extracellular matrix (dECM). The dECM was either seeded with human mesenchymal stem cells (hMSCs) as is or underwent further processing to form a hydrogel for 3D bioprinting (FIG. 18). The characterization of tunic dECM was achieved by FTIR, XRD, TGA, Raman spectroscopy, SEM and tensile mechanical analysis. After decellularization, the tunic dECM scaffold preserved the natural predesigned honeycomb-shaped microstructure, favorable for hMSCs attachment, proliferation, and chondrogenic differentiation. Both the tunic dECM scaffolds and bio-printed dECM constructs showed enhanced metabolic activity, cell proliferation and chondrogenic differentiation in vitro. This study offers a new potential source of dECM bio-ink scaffolds for tissue engineering and regenerative medicine.

The present data demonstrates that marine tunicates offer an excellent alternative source of rich ECM compared to the costly and risky disease contaminated land-animal derived sources. The cartilaginous tunic protein-cellulose complex extracted from Phallusia nigra grossly mimics the natural architecture of the cartilage microenvironment. Precise extraction of decellularized tunicate ECM and stable tunicate bio-ink are attained and explored for cartilage regeneration. The developed dECM scaffold and bio-ink displayed great mechanical and biochemical abilities, showing high cell attachment, proliferation and chondrogenesis.

Tunic forms the exoskeleton of Ascidians, commonly known as sea squirts or tunicates. Most of the solitary tunicates have thick, leathery, cartilaginous, smooth tunic with blood vessels. Tunicates form this supramolecular architecture with a cellulose-protein fiber complex in the tunic that is cemented by an acidic and sulfated polysaccharide [Chanthathamrongsiri, N., et al., The comparison of the properties of nanocellulose isolated from colonial and solitary marine tunicates. Heliyon, 2021. 7 (8): p. e07819]. These bioactive compounds provide mechanical rigidity and act as strategic defenses for tunicates survival in marine environment; and have a wide range of potential applications in the cellulose-based biomaterials industries and tissue engineering fields [Song, G., et al., Structure and composition of the tunic in the sea pineapple Halocynthia roretzi: A complex cellulosic composite biomaterial. Acta Biomater, 2020. 111: p. 290-301]. The tunicates especially P. nigra derived thick and smooth cartilaginous tunics (cellulose-protein complex) grossly mimic the natural architecture of human cartilage tissue. To obtain this potentially biomimetic tunic for cartilage tissue engineering application, an effective decellularization (pigment and cell-free) protocol was proposed [Govindharaj, M., et al., Bioprinting of bioactive tissue scaffolds from ecologically-destructive fouling tunicates. Journal of Cleaner Production, 2022. 330]. Decellularization procedure is essential to avoid the cellular and immunogenic materials for tissue engineering purpose. The tunicate-derived ECM materials show interesting functionalities such as capillarity, liquid absorption and thermostability which are important and essential features that were recently considered for tissue engineering in biomedical applications [Wysokowski, M., et al., Preparation of chitin-silica composites by in vitro silicification of two-dimensional Ianthella basta demosponge chitinous scaffolds under modified Stober conditions. Mater Sci Eng C Mater Biol Appl, 2013. 33 (7): p. 3935-41]. Recently, many studies focused on tunicates for the production of bioactive compounds from the species of Phallusia nigra. Today, cellulose-based materials have been used for various tissue engineering and biomedical applications from the species of Polycarpa reniformis [Arast, Y., et al., Selective Toxicity of Non Polar Bioactive Compounds of Persian Gulf Sea Squirt Phallusia nigra on Skin Mitochondria Isolated from Rat Model of Melanoma. Asian Pac J Cancer Prev, 2017. 18 (3): p. 811-818]; Phallusia nigra [Marhamati, Z., et al., Evaluation of the Physicochemical, Antioxidant, and Antibacterial Properties of Tunichrome Released from Phallusia nigra Persian Gulf Marine Tunicate. Journal of Food Quality, 2021. 2021: p. 1-11]; Halocynthia roretzi [Ramesh, C., et al., Marine Natural Products from Tunicates and Their Associated Microbes. Mar Drugs, 2021. 19 (6)]; Ciona intestinalis [Zhao, Y., C. Moser, and G. Henriksson, Transparent Composites Made from Tunicate Cellulose Membranes and Environmentally Friendly Polyester. ChemSusChem, 2018. 11 (10): p. 1728-1735] and functional cardiac patches from unidentified sea squirt [He, Y., et al., From waste of marine culture to natural patch in cardiac tissue engineering. Bioact Mater, 2021. 6 (7): p. 2000-2010]. The rapid growth rate and availability in aquaculture conditions [Lambert, G., et al., Wild and cultured edible tunicates: a review. Management of Biological Invasions, 2016. 7 (1): p. 59-66] make the tunicates as a renewable resource of tunic biomaterials.

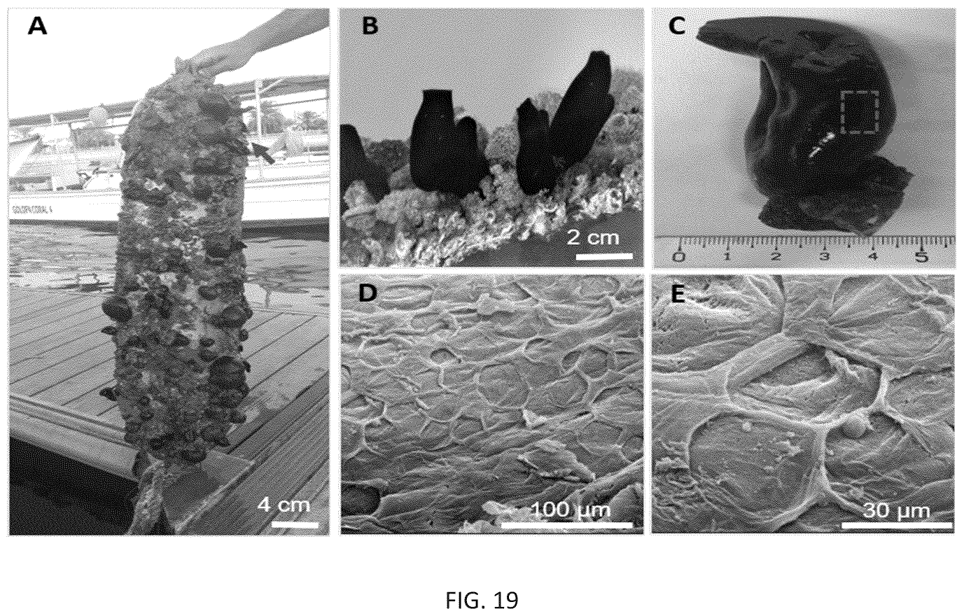

The present scientific interest, focusing on Phallusia nigra which has naturally fabricated unique structure possesses 3D-tunic fibrous skeleton that can reach 15.0 cm in length (FIG. 19). Notably, skeletons of marine tunicates, especially P. nigra, have high microporosity and intricate canal systems, as nature provides the most suitable microenvironments for cell infiltration, diffusion of cell nutrients, and waste removal [Govindharaj, M., et al., Bioprinting of bioactive tissue scaffolds from ecologically-destructive fouling tunicates. Journal of Cleaner Production, 2022. 3303; Mutsenko, V. V., et al., 3D chitinous scaffolds derived from cultivated marine demosponge Aplysina aerophoba for tissue engineering approaches based on human mesenchymal stromal cells. Int J Biol Macromol, 2017. 104 (Pt B): p. 1966-1974]. These are the essential features that are required for the fabrication of a unique tissue engineering scaffold. On the other hand, tunicate-derived cellulose hydrogel materials are reported for tissue engineering applications. For example, formulation of cellulose-based ECM bioink for regenerative medical applications using 3D printing technology [Govindharaj, M., et al., Bioprinting of bioactive tissue scaffolds from ecologically-destructive fouling tunicates. Journal of Cleaner Production, 2022. 330] and cellulose hydrogel from the crude extraction of sea derived tunic used as an excellent biocompatible material for cardiac tissue engineering applications. It has also been demonstrated that marine tunicate-derived materials can be used for wound dressing application [Govindharaj, M., et al., Bioprinting of bioactive tissue scaffolds from ecologically-destructive fouling tunicates. Journal of Cleaner Production, 2022. 330].

In the previous work, dECM hydrogel from the P. constellated, seeded with Mouse Embryonic Fibroblasts [Govindharaj, M., et al., Bioprinting of bioactive tissue scaffolds from ecologically-destructive fouling tunicates. Journal of Cleaner Production, 2022. 330] encouraged us to investigate the ability of P. nigra derived tunic dECM with hMSCs to support attachment, growth, and chondrogenic differentiation for cartilage tissue engineering applications. The decellularization and fabrication of ECM materials from this P. nigra is widely distributed in Abu Dhabi coastal water. This species is well-suited for practical use at the laboratory and industry levels.

First, the identification and detailed morphological characterization of the tunicate species is presented, followed by material characterization of the as-harvested, decellularized and lyophilized tunics dECM. Next, two different applications are described, namely bioactive tunic dECM scaffold for tissue engineering and, formulation of tunic dECM-based bioink for regenerative medicine applications using 3D bioprinting technology.

### Materials and Methods

Tunicates were collected from the Zayed Port, Abu Dhabi, UAE. The samples were thoroughly washed with DI water while stirring continuously and stored in 90% ethanol for species identification. The species was identified as Phallusia nigra and submitted in NCBI (Accession #MW990087).

Decellularization and Characterization of Extra Cellular Matrix Scaffold (dECM)

The decellularized tunic dECM from P. nigra was obtained following the protocol previously stated in recently published article (Govindharaj, Al Hashimi et al. 2022). The obtained tunic dECM microstructure was imaged using Quanta™ 450 FEG SEM. Further, analysis of the functional group of tunic dECM material was characterized using an Agilent 670-FTIR spectroscopy using KBr discs and collecting data from 400-4000 cm-1. Thermal stability of tunic dECM was measured by using Thermogravimetric analyzer SDT Q600 Instrument, in the air atmosphere. The crystallinity of dECM powder was assessed through X-ray diffraction (XRD) using Malvern Panalytical Empyrean 3 advanced instrument, conducted at room temperature from 2-400 (20). Cellulose derivatives in dECM materials were investigated using Raman spectroscopy using WITec alpha 300 equipment, with spectra recorded from 0-3500 using 600 mW laser. Using an Instron UTS-5965 instrument, a uniaxial tensile test was carried out with a load cell of 500 N and a crosshead speed of 40 mm/min.

### Cell Culture

Human mesenchymal stem cells (hMSCs) were obtained and were cultured in DMEM media (DMEM supplemented with 10% fetal bovine serum, 1% penicillin-streptomycin, and 2 mM L-glutamine) and maintained in an incubator (5% CO2, 37° C., and humidified atmosphere). The cells were trypsinized at 70% confluency and sub-cultured for further cell studies with both the tunic dECM scaffold and 3D bioprinted construct. The tunic dECM was sterilized overnight with 70% ethanol in a laminar airflow chamber, followed by UV sterilization for 3 hours in 24 well plates. Later, each scaffold was seeded with 200,000 cells for all the experiments.

Perfusion Method for Cell Seeding on dECM Scaffold

To perform the cell seeding on tunic dECM materials using perfusion methods, two syringes were connected with an elastic tube. Microporous tunic dECM scaffolds were placed in a 5 ml syringe and 2 ml of cell suspension (200000 cells/scaffold) were placed in another syringe. The syringe with cell suspension was gently ejected into the syringe with the dECM scaffold via the connecting tube. The dECM scaffold soaked with cells was kept in the syringe for 4 hours inside a CO₂ incubator. Later, the scaffolds were carefully transferred into 24-well plates with 0.5 ml of cell culture media.

### Bio-Inks Preparation and 3D Bioprinting

100 mg of tunic dECM powder was mixed with the 0.2% sodium alginate (SA) (Spectrum® Chemical MGF.CORP. Gardena, CA) to develop a 3D printable bioink. The bio-ink was prepared according to the same procedure previously described (Govindharaj, Al Hashimi et al. 2022). Then, the RegenHU 3D-Discovery™ Bioprinter (RegenHU Ltd, Switzerland) was used to print a simple 8×8×0.5 mm square mesh 3D construct. For 3D bioprinting, hMSCs were mixed with the developed tunic dECM/SA solution (100 mg of dECM/0.2% SA in 1.5 ml of DMEM). Bio-ink was loaded into an extrusion cartridge; the bioprinting was done with a nozzle of 0.25 mm diameter and pressure 0.5-0.6 MPa. After successful bioprinting (200,000 cells/construct), the construct was crosslinked with 200 mM CaCl2 for five minutes, followed by 15 minutes incubation with 0.2% FBS solution. After gentle washing with PBS solution, the 3D construct was incubated in the standard culture media (DMEM with 10% FBS and 1% penicillin-streptomycin) for further characterization.

### Cell Viability and Proliferation

To quantitatively analyze the metabolic activity of hMSCs cultured with P. nigra derived tunic dECM scaffold and the 3D bio-printed tissue constructs, an alamar blue (AB) assay (BioSource International, Camarillo, CA, USA) was performed as per the manufacturer's protocol, briefly described in previous work [Govindharaj, M., et al., Bioprinting of bioactive tissue scaffolds from ecologically-destructive fouling tunicates. Journal of Cleaner Production, 2022. 330; Govindharaj, M., U. K. Roopavath, and S. N. Rath, Valorization of discarded Marine Eel fish skin for collagen extraction as a 3D printable blue biomaterial for tissue engineering. Journal of Cleaner Production, 2019. 230: p. 412-419]. The viability of hMSC within dECM scaffold and bio-printed constructs were performed according to the manufacturer's protocol also evaluated by LIVE/DEAD staining with Calcein AM and Ethidium homodimer1 (LIVE/DEAD™ Viability/Cytotoxicity Kit, Thermo Fisher Scientific, USA)

### Chondrogenic Differentiation

For chondrogenic differentiation, hMSCs loaded dECM scaffold and cell-encapsulated 3D bio-printed constructs were cultured in standard DMEM medium supplemented with 10 μmM dexamethasones (Sigma-Aldrich), 0.2 mM ascorbic acid (Sigma-Aldrich), 10 μg/ml insulin, 5.5 μg/ml transferrin, 6.7 ng/ml selenous acid (ITS; Gibco Life Technologies), 10 ng/ml transforming growth factor (TGF)-β3 (Miltenyi Biotec), 100 U/ml penicillin and 100 μg/ml streptomycin. The medium without chondrogenic induction media was considered as the control. The media was replaced every 3 days. Samples were collected at days 14 and 21 to assess the chondrogenic differentiation through histological staining Alcian Blue and Safranin O.

### Histological Analysis

To assess the deposition of glycosaminoglycans (GAGs) on tunic dECM scaffold and 3D bio-printed constructs that had been cultured for 14 and 21 days in the chondrogenic medium were stained with alcian blue and safranin O [Aleksander-Konert, E., et al., In vitro chondrogenesis of Wharton's jelly mesenchymal stem cells in hyaluronic acid-based hydrogels. Cell Mol Biol Lett, 2016. 21: p. 11]. After removal of the culture media, the cell-seeded dECM scaffold, and 3D constructs were fixed in 4% paraformaldehyde for 30 min, then washed twice with PBS and 0.1% stock solutions of alcian blue and safranin O solution were added and incubated for 30 min at room temperature. Then, the dye solution was removed and the dECM scaffold and 3D constructs were washed gently with distilled water and observed and imaged under a Nikon Eclipse TS100 inverted microscope.

### RNA Isolation and Real-Time PCR Analysis

The RNA from cultured cells was isolated using a Qiaquick RNA extraction kit (Qiagen) according to the manufacturer's instructions. The extracted RNA was quantified using a Nanodrop ND-1000 spectrophotometer (Nanodrop Technologies, Wilmington, DE). 1 uG of mRNA was reverse transcribed into cDNA using superscript IV VILO cDNA Synthesis Kit (Catalogue number 11766050, Invitrogen, Thermo Fisher Scientific). Real-time PCR reactions were carried out in triplicates with 500 ng cDNA template per reaction using PowerUp SYBR Green Master Mix (Catalogue number A25776, Thermo Fisher Scientific) in a Steponeplus Real-Time PCR System (Applied Biosystems). The PCR reactions were set up at 10 μl volume per well. The thermal conditions used were as follows: 50° C. for 2 min; 95° C. for 2 min; 40 cycles of 95° C. for 15 sec; 53° C. for 15 sec; 72° C. for 1 min with a standard ramp rate. The sequences of the forward and reverse primers of genes analyzed were adapted from Hoyer et. al., 2013.

### Results

### Morphological Analysis of Solitary Marine Tunicate

Adult tunicate specimens were collected from marine boat floats (FIG. 19A) and were identified as Phallusia nigra (NCBI Accession #MZ736873). The tunicate species was observed with velvety black and dark brown-colored sac-shaped bodies with separate water entrance and exit tubes (siphons), which belong to marine solitary ascidian as seen in FIGS. 19B-19C. FIGS. 19D-19E demonstrates the SEM micrographs of the honeycomb-shaped external surface of tunicates that support and protect the body.

Preparation of Tunic dECM Scaffolds from P. nigra

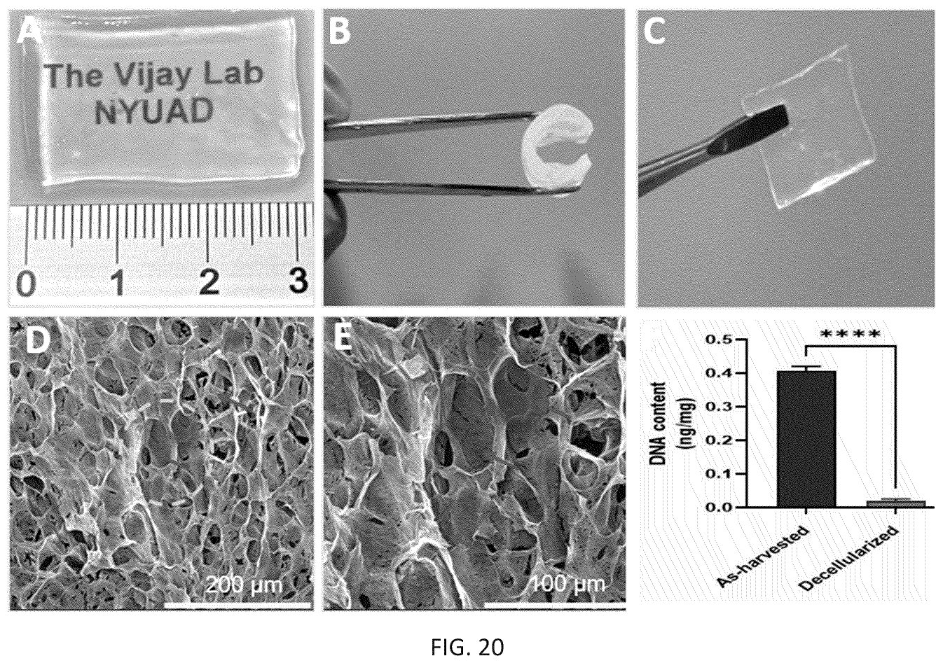

After step-by-step treatment of P. nigra derived tunic dECM material with decellularization solutions, a translucent material was produced. FIG. 20A demonstrates the extent of translucency by placing the decellularized tunic in front of text and showing it is see through with little to no effect on the text. FIG. 20B-20C show how the thick translucent leathery and flexible scaffold materials look similar to human cartilage tissue. The tunic materials preserve the naturally predesigned honeycomb-shaped structure after decellularization. This might reflect that, the decellularization procedure did not affect the natural architecture of tunicates that mimic the architecture of human cartilage, due to its high interconnected microporous structure (indicated by red arrows) and 3D topology FIG. 20D-FIG. 20E. The tunic tissue was decellularized by freezing-thawing, followed by Triton X-100 treatment. After decellularization of tunic, the total content of DNA was quantified to validate the cellular content in the decellularized tunic material. The DNA content of the decellularized tunic tissue revealed a significant decrease compared with the native tunic tissue, as measured by the dsDNA content was less then 0.1 ng (p≤0.0005; FIG. 20F).

Formulation of Bioink from P. nigra Derived dECM

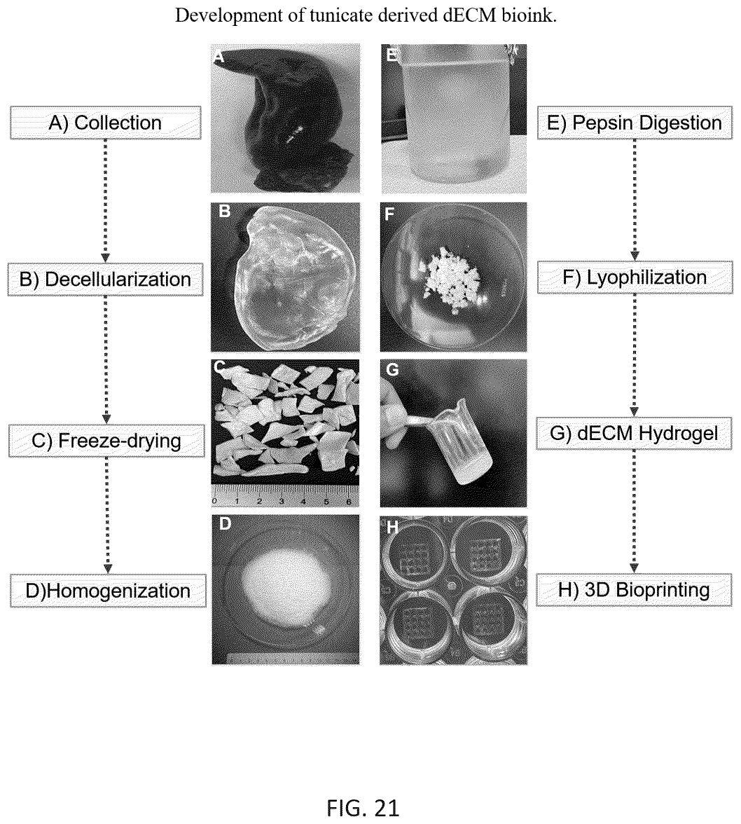

Several steps are involved in the fabrication of dECM bio-ink for 3D bioprinting (FIG. 21). The first step is decellularization of tunic tissue removal of cellular and genetic component. Then the decellularized dECM scaffold materials was freeze-dried. and homogenization. Further, the freeze-dried materials were homogenized and pepsin digested with 0.5 M acetic acid. Then, tunic dECM solution was centrifuged at 4000 rpm for 20 min to remove undissolved large particles. Any particles that remain in the tunic dECM bio-ink can block the nozzle of the 3D cell printing system during the printing process. The removal of large particles is therefore an important step in tunic dECM bio-ink production. Finally, the lyophilized powder was transferred into the bio-ink solution for 3D bioprinting (FIG. 21). To develop a 3D printable tunic dECM bio-ink that can be extruded through the dispensing modules of the 3D bioprinting system. At last, the tunic dECM solution was neutralized to obtain the final form of the tunic dECM bio-ink for cell encapsulation and the 3D bioprinting process.

Characterization of Tunic dECM Materials

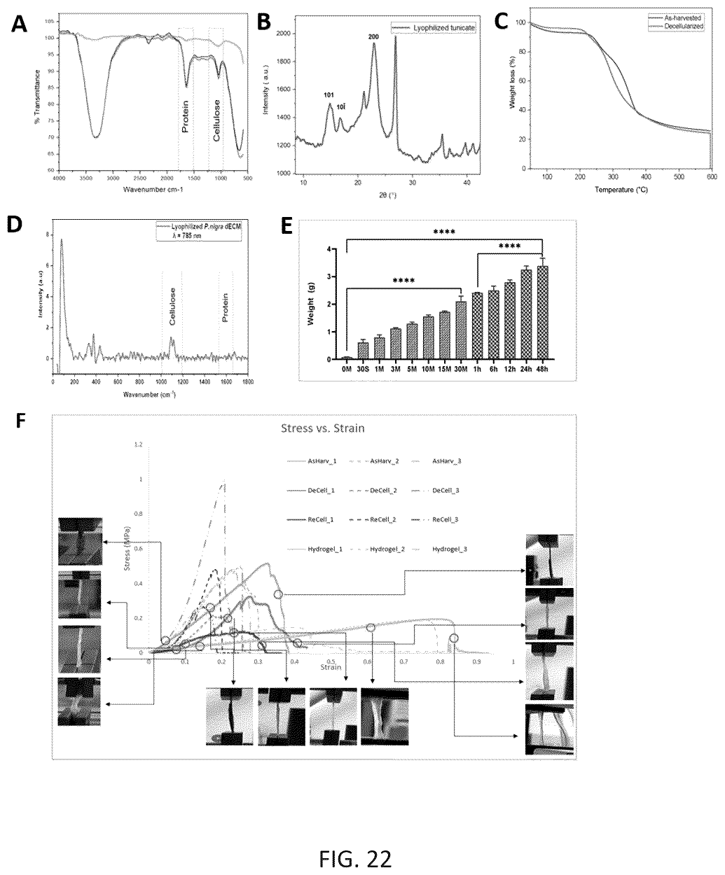

Further, investigate the composition of tunic dECM materials, FTIR analyses was performed. The spectra of as harvested, decellularized and rewetted samples are shown (FIG. 22A). The strong bands at 1647 and 1542 cm 1 represent amide I & II respectively, indicating the presence of protein [Song, G., et al., Structure and composition of the tunic in the sea pineapple Halocynthia roretzi: A complex cellulosic composite biomaterial. Acta Biomater, 2020. 111: p. 290-301]. Whilst, the characteristic peaks for cellulose can be observed at 1036, 1061, 1115, and 1163 cm 1 in the region of 1100 cm 1. This strongly reflects that tunic dECM materials did not lose the bioactive cellulose and protein after the decellularization.

Raman spectroscopy measurements confirmed the presence of cellulose and protein complex in the tunic dECM materials. The spectra acquired from the tunic dECM materials show clear peaks at 379 cm⁻¹, 1095 cm⁻¹, and 1122 cm⁻¹, which are the characteristic peak for cellulose (FIG. 22D). [Song, G., et al., Structure and composition of the tunic in the sea pineapple Halocynthia roretzi: A complex cellulosic composite biomaterial. Acta Biomater, 2020. 111: p. 290-301; Ulman, A., et al., A massive update of non-indigenous species records in Mediterranean marinas. PeerJ, 2017. 5: p. e3954]. Also, peaks in the region of 1000 and 1700 cm-1 were absorbed to analyses protein in dECM materials. The peaks at 1031 cm-1 confirm the presence of phenylalanine in proteins, and amide⁻¹ band component assignment to protein secondary structure was observed at 1673 cm⁻¹ [Cheng, Y., et al., Study on the Anti-Biodegradation Property of Tunicate Cellulose. Polymers (Basel), 2020. 12 (12]. This data further confirms the phenylalanine in collagen at 1032 cm-1 in the composition of tunic dECM materials.

X-ray diffractogram of the powdered (decellularized) lyophilized tunic dECM (FIG. 22B) further confirms that the tunic cellulose is highly crystalline. Tunic dECM materials show the peak values at 20=22.9°, 16.6° and 14.8° for the characteristic (002), (101) and (101) lattice planes, respectively. These results confirm the presence of cellulose with good crystalline structure, which is the good agreement with previously published literature [Govindharaj, M., et al., Bioprinting of bioactive tissue scaffolds from ecologically-destructive fouling tunicates. Journal of Cleaner Production, 2022. 330; Kale, R. D., P. S. Bansal, and V. G. Gorade, Extraction of Microcrystalline Cellulose from Cotton Sliver and Its Comparison with Commercial Microcrystalline Cellulose. Journal of Polymers and the Environment, 2017. 26 (1): p. 355-3643, 17].

The TGA thermogravimetric analysis revealed the thermal stability of lyophilized both as-harvested and decellularized tunic, which are shown in FIG. 22C. The onset temperature of both tunics materials is around 220° C., which agrees with the previous report on the TGA thermogram range of tunicate-derived cellulose/cellulose nanocrystals [June, S. Y., et al., Tunicate Cellulose Nanocrystals as Stabilizers for PLGA-based Polymeric Nanoparticles. Biotechnology and Bioprocess Engineering, 2020. 25 (2): p. 206-214]. In the temperature range of 250-400° C., more than 70% weight loss occurred. This range is well within the 280 to 400° C. temperature associated with the cellulose thermal decomposition [Moon, R. J., et al., Cellulose nanomaterials review: structure, properties and nanocomposites. Chem Soc Rev, 2011. 40 (7): p. 3941-94]. The thermal degradation range of both the decellularized and the as-harvested tunic completed at around 250-400° C. It should be also mentioned that there was a slight difference was observed, (decomposition temperature extended by 20-40° C. for as-harvested samples) in the as harvested sample due to the presence of inorganic salts and heavy metal ions in the as-harvested tunicates because of their living environment [Zhao, Y. and J. Li, Excellent chemical and material cellulose from tunicates: diversity in cellulose production yield and chemical and morphological structures from different tunicate species. Cellulose, 2014. 21 (5): p. 3427-3441].

The swelling behavior of lyophilized dECM tunic showed (FIG. 22E) the interesting functional characteristic of capillarity. Considering, interconnected 3D microporous and capillary-like nature of the nanocellulose fibril network in the dECM tunic possesses very high fluid-absorbing and retaining capabilities based on time-dependent manner, providing reliable evaluation. The results proving the capability of the lyophilized tunics to be used as tissue engineering scaffolds provide a favorable 3D microenvironment to the cells, in terms of cell infiltration, attachment, migration, growth, diffusion of cell nutrients and growth factors, and removal of waste [Govindharaj, M., et al., Bioprinting of bioactive tissue scaffolds from ecologically-destructive fouling tunicates. Journal of Cleaner Production, 2022. 330; Heise, K., et al., Nanocellulose: Recent Fundamental Advances and Emerging Biological and Biomimicking Applications. Adv Mater, 2021. 33 (3): p. e2004349]

FIG. 22F shows the stress vs. strain graph in the tensile test of as-harvested, decellularized, and recellularized tunic of P. nigra as well as hydrogels. From the analysis of the tensile test data, the modulus of as-harvested samples was about 2.39 MPa which is less than decellularized samples as well as recellularized samples with the modulus of 3.61 MPa and 2.92 MPa, respectively. Moreover, the hydrogel samples were also found to possess a modulus of about 0.47 MPa.

Biocompatibility of hMSCs on dECM Scaffold and 3D Bio-Printed Construct

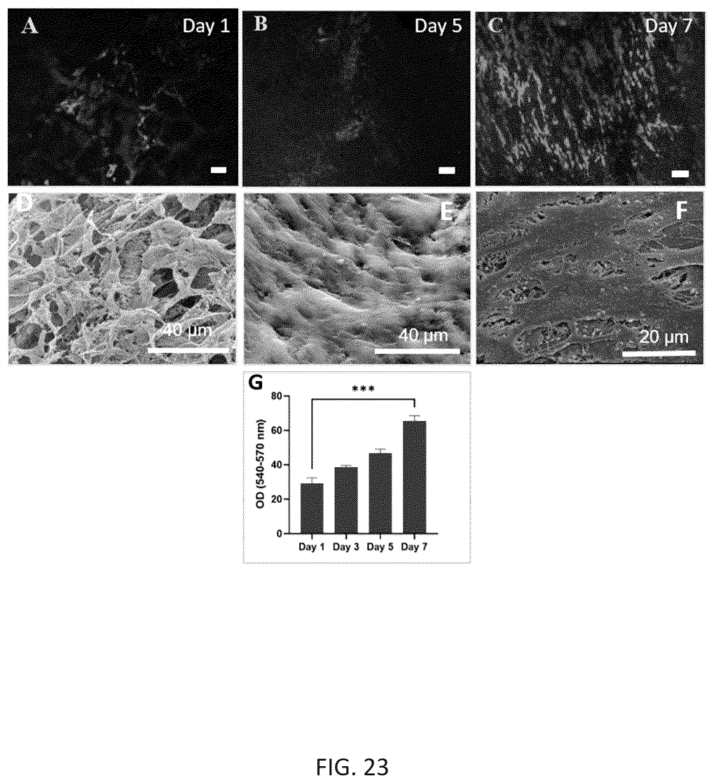

Biocompatibility tests are important to prove the developed dECM materials are cell-friendly. FIG. 23 shows that process involved in the decellularization and, preparation has no negative impact on cell viabilities and properties instead supports cell attachment, proliferation, and differentiation. The viability of hMSCs cultured on tunic dECM scaffolds was monitored for seven days. LIVE/DEAD imaging (green: viable cells, red: dead cells) confirmed that no toxic effect was observed from the dECM scaffolds on hMSCs at all time points of investigation (FIG. 23). Moreover, the image indicates that there is no necrotic cells death in the inner part of dECM scaffolds after seven days in culture, evidence for scaffold infiltration through interconnected honeycomb-like pores. The hMSCs growth was analyzed by attachments, proliferation, and gradually covering of the tunic dECM materials through infiltration and infilling via pores cell mass. On day seven, the result suggested that the interconnected honeycomb-porosity of the tunic dECM scaffold promotes cell ingrowth and surface proliferation by making cells sheet patterns (FIG. 23B). Cultivation of HMSCs on dECM scaffold made cell pattern to ensure that cell sheet formation and orientations within the scaffold (FIG. 23C). FIGS. 23D-F demonstrates SEM micrographs of dECM support attachments, proliferation, and differentiation of hMSCs in a time-dependent manner. On day 1 (FIG. 23D) cell attachment was observed on the scaffold, whereas on, hMSCs were then bioprinted into tunic dECM/Alginate bio-inks, respectively. The live & dead assay confirmed that hMSCs encapsulated 3D construct maintained extremely high cell survival rates at all time points of the investigation (FIG. 23A-C). Besides, confocal laser scanning microscopy (CLSM) images further showed that well-live cells were evenly distributed throughout the 3D printed constructs. Notably, on day 7 extremely high cell survival rate was maintained with an increased cell number (FIG. 23C). On day 7 (FIG. 23F) dECM scaffolds were completely covered with cell sheets.

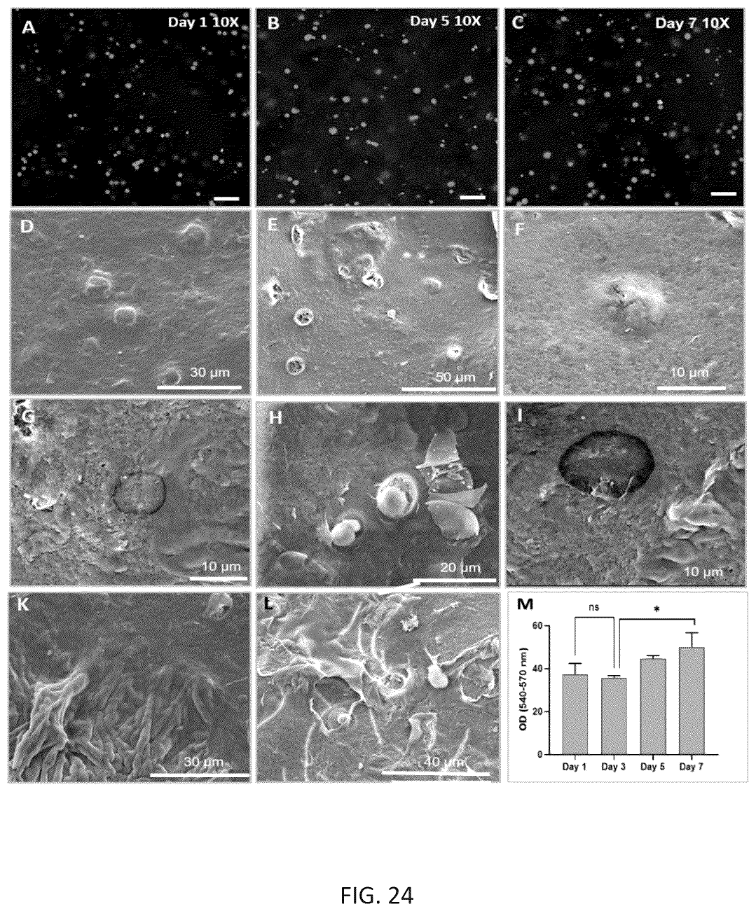

Hence, these results confirmed the successful development of dECM/alginate bio-inks and the feasibility of the 3D bioprinting method. The SEM images (FIG. 24 D-L) provide reliable insight into the surface and internal morphology of hMSCs penetration, migration, and proliferation. The hMSCs grew and paved on 3D bio-printed construct, the cell was well attached and eventually distributed on a 3D construct and the pseudopod was found to be sticking out FIG. 24.

### Histological Analysis Chondrogenic Differentiation

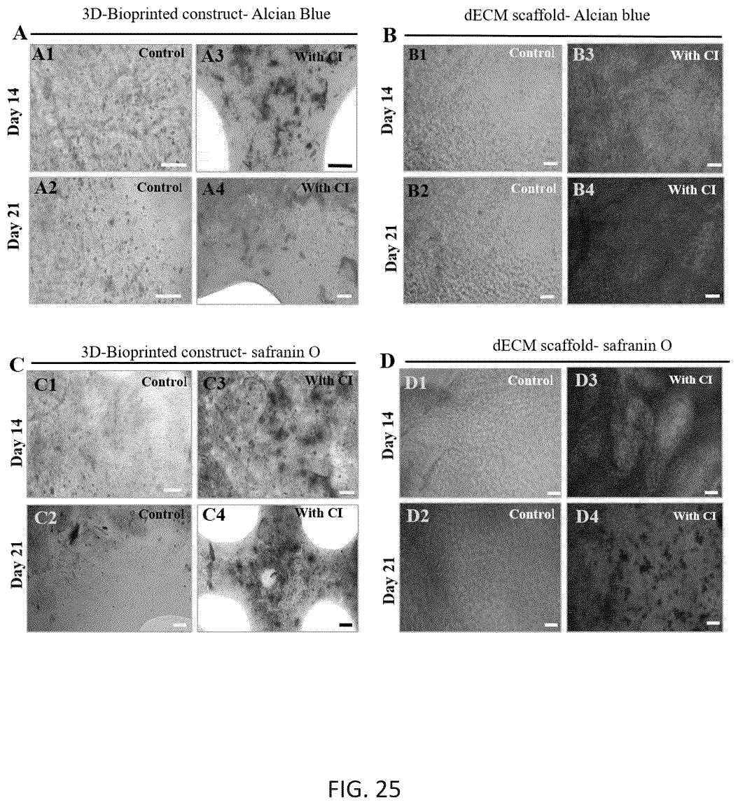

To investigate the potential of tunic dECM scaffold as a 3D printed construct for supporting chondrogenic-specific tissue formation, it was examined via histological staining. The presence of chondrogenic media to increase the cell differentiation from the first to third week is much more evident if compared with the same materials without chondrogenic induction media. These observations were strongly confirmed by the Alcian Blue staining that highlighted any outstanding difference between the samples in expansion medium (FIGS. 25 A1 & A2) and the samples in chondrogenic medium (FIGS. 25 A3 & A4), in terms of extracellular matrix nature. In the expansion medium, the blue staining did not reveal any GAG deposition, whereas the presence of induction factors in the culture medium led to the deposition of cartilage extracellular matrix GAG (with CI). On day 14, amount of extracellular matrix deposition is bigger (FIG. 25 A3) and becomes more and more reliable at day 21 (FIG. 25 A4). In addition, the differentiated mesenchymal cells in 3D constructs were strongly evident in the chondrogenic differentiation through Safranin O staining (FIGS. 25 C3 & C4). Both intense Alcian Blue and safranin O staining confirm the cartilage-like nature of the newly deposited GAG around the 3D bioprinted construct. The deposition of GAG on the dECM scaffold combined with Alcian blue was colored blue and appears red in combination with Safranin O when observed by microscopy. After days 14 and 21, the color gradually deepened in a time-dependent manner (FIG. 25B-D). On day 21, the color of dECM material was the strongest, whereas the control dECM materials were the weakest (FIGS. 25 B1, B2, C1, and C2). The results indicating GAG deposition by chondrogenic induction of hMSCs were increased with time and the proportion of dECM, which was elevated both in dECM scaffold and 3D bioprinted construct.

Gene Expression Analysis of Chondrogenic Differentiation of hMSC's on 3D Bioprinted and dECM Scaffolds

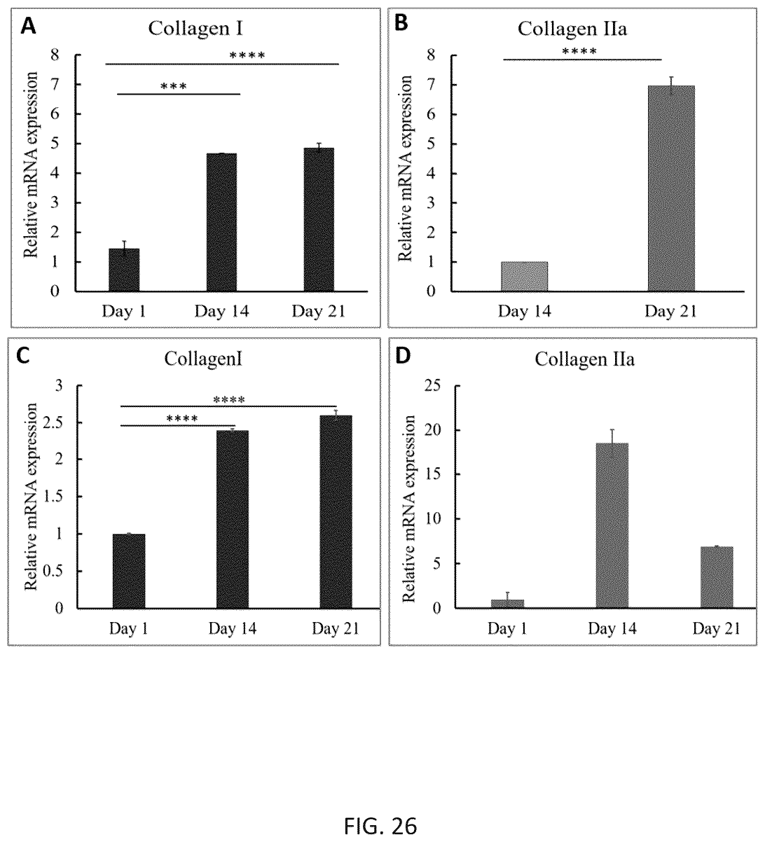

FIG. 26 demonstrates the PCR analysis of collagen I and collagen IIa gene expression in chondrogenically stimulated and non-stimulated control samples. Collagen I and Collagen IIa gene expression in bioprinted hMSCs indicating chondrocyte differentiation. The Collagen I showed significant expression on days 14 and 21 compared to day 1 (FIG. 26A). Collagen IIa started to express on day 14 and showed ˜7-fold expression by day 21 (FIG. 26B). The gene expression of hMSCs cultured on the dECM scaffold showed significant expression on days 14 and 21 compared to day 1 (FIG. 26C). Collagen IIa peaked expression on day 14, in contrast, on day 21 the expression was declined (FIG. 26D). The gene expression of the housekeeping gene GAPDH was determined to verify the usage of equal amounts of RNA for RT-PCR. The data reported as mean+/−SD (n=3 in triplicate; *** p<0.001, **** p<0.00001 by two-tailed students t-test between test and control samples. (FIG. 26A&B).

### Discussion

In the disclosed example, shown is a chondrogenic potential of bioactive tunic dECM scaffold and bioink from marine fouling solitary tunicates, identified to be Pallusia nigra (NCBI Accession #MZ736873). P. nigra is a putative cosmopolitan ascidian that is common in harbors and embayment areas, lives in shallow water, and is attached to the hard surface such as dead coral, pier pilings, floats, bottom of the ship, hull, and other marine structures in the port (FIG. 19A, 19B). P. nigra is typically a velvety black and brown (FIG. 19B, 19C) and has a thick and smooth outer coat called tunic [Chanthathamrongsiri, N., et al., The comparison of the properties of nanocellulose isolated from colonial and solitary marine tunicates. Heliyon, 2021. 7 (8): p. e07819]. The species identified on the coast of Abu Dhabi showed similar morphological features as other P. nigra species reported from the Indo-Pacific Ocean (India, Japan, and Hawaii) and in the Mediterranean Sea [Ulman, A., et al., A massive update of non-indigenous species records in Mediterranean marinas. PeerJ, 2017. 5: p. e3954] The external morphology of the as-harvested tunic from P. nigra was investigated through SEM analysis. FIG. 19D, E shows the tunicate surface possesses an evolutionarily unique design that resembles the honeycomb-shape.

Preservation of intact tunic ECM compositions (cellulose and protein) is crucial to mimic the native microenvironment for cartilage regeneration [Lim, T., et al., A decellularized scaffold derived from squid cranial cartilage for use in cartilage tissue engineering. J Mater Chem B, 2020. 8 (20): p. 4516-4526]. FTIR and Raman spectroscopy analysis confirmed the typical bands of cellulose and protein in the tunic dECM materials after decellularization (FIG. 22A, 22D). The tunic is a composition of nanoscale cellulose microfibrils in a protein matrix that was shown to improve the biological mechanical properties of dECM materials. The microstructure of tunic dECM powder through XRD analysis revealed cellulose nanocrystals on ECM matrix [Kaur, K., et al., Injectable chitosan/collagen hydrogels nano-engineered with functionalized single wall carbon nanotubes for minimally invasive applications in bone. Mater Sci Eng C Mater Biol Appl, 2021. 128: p. 112340]. The effect of the cellulose nanocrystals in tunic dECM provide thermal stability indicated in FIG. 22C, as hydrogels with cellulose crystalline structures are known to have higher mechanical properties [Mariia, K., et al., Novel chitosan-ulvan hydrogel reinforcement by cellulose nanocrystals with epidermal growth factor for enhanced wound healing: In vitro and in vivo analysis. Int J Biol Macromol, 2021. 183: p. 435-446]. The P. nigra derived tunic dECM/alginate hydrogel samples were also found to possess a modulus of about 0.47 MPa (FIG. 22F).

After, step-by-step decellularization treatment of P. nigra tunic tissue with alkali-acidic solutions, microporous tunic 3D tunic dECM scaffolds was developed. The microstructure and internal composition of decellularized P. nigra tunic dECM scaffold was characterized through SEM analysis. As can be seen from FIGS. 20D and E, the tunic dECM scaffold preserves the naturally predesigned honeycomb-shaped structure with improved interconnected porosity even after decellularization (indicated by red arrows) (FIG. 20D-E). After decellularization, the tunic dECM scaffold revealed low levels of ds-DNA contents compared to native tissue, as measured by the dsDNA content (FIG. 20F). Removal of DNA content in dECM is an important criterium because DNA is directly correlated with adverse host reactions [Lim, T., et al., A decellularized scaffold derived from squid cranial cartilage for use in cartilage tissue engineering. J Mater Chem B, 2020. 8 (20): p. 4516-452623; Visscher, D. O., et al., A photo-crosslinkable cartilage-derived extracellular matrix bioink for auricular cartilage tissue engineering. Acta Biomater, 2021. 121: p. 193-203]. In the present study, due to abundant cellulose content, tunic dECM was greatly preserved and showed great potential in biomechanics.

Dynamic seeding methods using perfusion systems have enhanced the uniform cell seeding around the dECM scaffold. Cell infiltration on tissue scaffolds during in vivo implantation is limited and is a major concern due to the lack of porosity [Novak, T., et al., In Vivo Cellular Infiltration and Remodeling in a Decellularized Ovine Osteochondral Allograft. Tissue Eng Part A, 2016. 22 (21-22): p. 1274-1285]. Whereas, tunic dECM scaffold could facilitate uniform cell infiltration due to its honeycomb-like interconnected porosity to facilitate cell infiltration, nutrient supply, vascularization, resulting in tissue ingrowth on the surface and cell growth on the inside. FIG. 23 shows the gradual covering of the dECM surface (FIG. 23A) via concentric cell infiltration of honeycomb-like micropores (FIG. 23B) to completely cover the tunic dECM scaffold with hMSCs cell layers (FIG. 23C). The naturally derived materials for stem cell-based tissue engineering application should support cell viability [Place, E. S., N. D. Evans, and M. M. Stevens, Complexity in biomaterials for tissue engineering. Nat Mater, 2009. 8 (6): p. 457-70] via their physicochemical and mechanical properties [Zhao, X., et al., Applications of Biocompatible Scaffold Materials in Stem Cell-Based Cartilage Tissue Engineering. Front Bioeng Biotechnol, 2021. 9: p. 60344429; Engler, A. J., et al., Matrix elasticity directs stem cell lineage specification. Cell, 2006. 126 (4): p. 677-89]. HMSCs were observed to attach, spread and align along the dECM scaffold in a time-dependent manner. A single-cell attachment was found on day 1 (FIG. 23D), whereas, dECM scaffolds were completely covered with cell mass on day 7 (FIG. 23F), indicating cell migration and proliferation along the dECM scaffold. A similar tendency was also reported in a previous work, involving dECM materials derived from tunicate P. constellatum [Govindharaj, M., et al., Bioprinting of bioactive tissue scaffolds from ecologically-destructive fouling tunicates. Journal of Cleaner Production, 2022. 330]. Tunic cellulose-derived self-conductive natural scaffolds as functional cardiac patches and successfully demonstrated their biocompatibility with cardiomyocytes in vitro [He, Y., et al., From waste of marine culture to natural patch in cardiac tissue engineering. Bioact Mater, 2021. 6 (7): p. 2000-2010]. Blue assay revealed a two-fold increase in hMSCs metabolic activity on day 7, indicating that the metabolic activity of hMSCs cells was not affected by the dECM scaffold and its decellularization procedure, supporting cell proliferation.

Furthermore, the study investigated whether the tunicates-derived dECM bioink can be utilized for the 3D bioprinting technique for cartilage regeneration. FIG. 21 shows the step-by-step process involved in the preparation of dECM bioink from tunicate (P. nigra) dECM materials. For the 3D bioprinting, the tunicate derived dECM powder was used with alginate for the formulation of bio-ink based on the recently published work [Govindharaj, M., et al., Bioprinting of bioactive tissue scaffolds from ecologically-destructive fouling tunicates. Journal of Cleaner Production, 2022. 330]. In the developed dECM/alginate bioink formulation, the supporting component provided initial structural integrity and proper printability. Also, the tunic dECM was crosslinked with CaCl₂) to make a stable 3D construct. The natural derived hydrogel materials crosslinked with CaCl₂) improved the structural integrity to the 3D bioprinted dECM/alginate constructs [Rasheed, A., et al., Extrusion-Based Bioprinting of Multilayered Nanocellulose Constructs for Cell Cultivation Using In Situ Freezing and Preprint CaCl2 Cross-Linking. ACS Omega, 2021. 6 (1): p. 569-578; Lee, J., et al., Bone-derived dECM/alginate bioink for fabricating a 3D cell-laden mesh structure for bone tissue engineering. Carbohydr Polym, 2020. 250: p. 116914]. This dECM/alginate-based bioink was 3D bioprintable, and the printed constructs were structurally and mechanically stable and possess a proper 3D microenvironment for cells migration, proliferation, and chondrogenic differentiation. These are the significant functionalities for an ideal bioengineered 3D construct that can be used for cartilage reconstruction [Visscher, D. O., et al., A photo-crosslinkable cartilage-derived extracellular matrix bioink for auricular cartilage tissue engineering. Acta Biomater, 2021. 121: p. 193-203].

In addition, the hMSCs in the 3D bioprinted tunic dECM/alginate maintained higher cell viability and proliferation demonstrated through LIVE/DEAD assay. The hMSCs cultured on 3D bio-printed construct were monitored for 7 days (FIG. 24A-C). The overwhelming majority of viable cells at all time points indicate that the tunic dECM/alginate 3D hydrogel construct provides a favorable microenvironment for hMSCs proliferation. Further, SEM analysis reveals the ability of hMSCs to penetrate, adhere and proliferate into the 3D hydrogel construct. In particular, hMSCs encapsulated into the dECM construct were bulked on the surface during day one (FIG. 24D-F), moreover, cells started to invade outside (FIG. 24G-I) as well as the inner part of the 3D construct, penetrating deeply inside and out (FIG. 24J), which results in envelopment by a cell sheet and gradually invade the interior part of scaffold and 3D constructs (FIG. 24K-L). Also, hMSCs proliferation was declined on day 7 due to the encapsulation of hMSCs in artificial environment, on day 7, the metabolic activity was significantly increased compare to day 3 (FIG. 24M), indicate favorable microenvironment.

hMSCs are known for their extraordinary differentiation potential and unique therapeutic capacity through fine immunomodulation and paracrine signaling [Wang, Y., et al., Plasticity of mesenchymal stem cells in immunomodulation: pathological and therapeutic implications. Nat Immunol, 2014. 15 (11): p. 1009-1633; Yao, Y., et al., Paracrine action of mesenchymal stem cells revealed by single cell gene profiling in infarcted murine hearts. PLOS One, 2015. 10 (6): p. e0129164]. Notably, hMSCs can maintain the morphological phenotype of human chondrocytes and boost the cartilage ECM production in both dECM scaffold and 3D bioprinted construct, confirmed by histological staining such as Alcian blue and Safranin O staining (FIG. 25). To test the ability of tunic dECM scaffold and dCM/alginate 3D constructs for supporting hMSCs, chondrogenic differentiation potential was performed. FIG. 26 demonstrates the PCR analysis of collagen I and collagen IIa gene expression in chondrogenically stimulated and non-stimulated control samples.

Both dECM and 3D printed scaffold supported the chondrogenic differentiation confirmed by gene expression analysis. The Collagen I showed significant expression on days 14 and 21 compared to day 1. Collagen IIa started to express on day 14 and showed ˜7-fold expression by day 21 in bioprinted hMSCs indicating chondrocyte differentiation (FIG. 26A, B). The gene expression of hMSCs cultured on the tunic dECM scaffold showed significant expression on days 14 and 21 compared to day 1 (FIG. 26C). Collagen IIa peaked expression on day 14 and declined on day 21 (FIG. 26D). probably due to the conversion of the mRNA to protein and relatively less stability of collagen II transcripts compared to the collagen I transcripts [Dozin, B., et al., Stabilization of the mRNA follows transcriptional activation of type II collagen gene in differentiating chicken chondrocyte. Journal of Biological Chemistry, 1990. 265 (13): p. 7216-7220]. The gene expression profile can vary depending on the mechanical and chemical cues, cells are getting from the tissue microenvironment, which is evident from the difference in the gene expression pattern observed in the dECM scaffolds and bioprinter tissues.

dECM materials derived from the marine tunicates bear certain valuable advantages compared to terrestrial animal-derived materials for cartilage tissue engineering. Till today, there are zero reports on transmitting disease from marine animals to humans, whereas dECM materials from land animals are sometimes immunogenic, costly, and carry the potential risk of transmissible diseases such as bovine spongiform encephalopathy and foot and mouth disease to human beings [Lim, T., et al., A decellularized scaffold derived from squid cranial cartilage for use in cartilage tissue engineering. J Mater Chem B, 2020. 8 (20): p. 4516-4526]. On the other hand, the use of biomaterials from either bovine or porcine is a major concern in Islam, Hinduism, and Judaism due to religious restrictions [Lim, T., et al., A decellularized scaffold derived from squid cranial cartilage for use in cartilage tissue engineering. J Mater Chem B, 2020. 8 (20): p. 4516-4526]. Therefore, it has been of great interest to seek alternative sources for the extraction of biomimetic dECM materials. Based on these in-vitro results the use of tunic dECM is very promising for cartilage tissue regeneration, and further in vivo studies will be performed to progress the work further.

Based on the performance of tunic dECM scaffold and 3D printed bio-inks for cartilage tissue engineering a clearer picture of this bioactive material is emerging. The scaffold and bio-ink from the marine tunicate P. nigra were successfully developed for cartilage tissue engineering application. Both materials provided cartilage-specific microenvironment, good biocompatibility and chondrogenic differentiation in vitro. The dECM provided desirable 3D printability, structural and mechanical stability and mimicked the architecture of cartilage tissue, which is favorable for hMSCs proliferation and maturation in 3D bioprinted constructs. These features make the dECM scaffold and bio-ink developed in this work a novel and attractive model for stem cell-based cartilage regeneration.

### Example 4: 3D (Bio) Printed Tri-Layered Cellulose/Collagen-Based Drug Eluting Fillers for Treating Deep Tunneling Wounds

The skin is the body's largest organ and protects the internal organs from external invasions [Zaid, N. A. M., et al., Promising Natural Products in New Drug Design, Development, and Therapy for Skin Disorders: An Overview of Scientific Evidence and Understanding Their Mechanism of Action. Drug design, development and therapy, 2022. 16: p. 23]. Upon injury, several complex and dynamic processes ensue including inflammation, hemostasis, and maturation by cell proliferation [Muthusamy, S., et al., 3D bioprinting and microscale organization of vascularized tissue constructs using collagen-based bioink. Biotechnology and Bioengineering, 2021. 118 (8): p. 3150-3163]. Among those injuries, tunneling wounds are the most difficult ones to treat as these wounds create passageways underneath the skin surface, that can be shallow or deep, short or long, and can take twists and turns. The tunnels could extend to form a full-thickness wound into and through the soft tissue of the subcutaneous muscle. The causes of tunneling wounds are many including infections associated with normal wounds, abscess formation, stalled healing, tunneling due to shear forces and pressure on the skin such as pressure ulcers, comorbidities such as diabetes and prolonged use of drugs such including antibiotics, and corticosteroids [Rosenbaum, A. J., et al., Advances in wound management. JAAOS-Journal of the American Academy of Orthopaedic Surgeons, 2018. 26 (23): p. 833-843]. Available wound care solutions only cater superficial or surface wounds and the risk of untreated tunneling wounds poses major health concerns [Sood, A., M. S. Granick, and N. L. Tomaselli, Wound dressings and comparative effectiveness data. Advances in wound care, 2014. 3 (8): p. 511-529].

While there are no ideal treatment methods for tunneling wounds, several attempts had been made to use acellular dermal matrix, biopolymer- or hydrogel-based formulations, either in a sheet-form or paste form [Kim, Y. H., et al., A Prospective Randomized Controlled Multicenter Clinical Trial Comparing Paste-Type Acellular Dermal Matrix to Standard Care for the Treatment of Chronic Wounds. Journal of Clinical Medicine, 2022. 11 (8): p. 2203]. While the sheet form is a traditional wound dressing used for treating superficial wounds that are rolled and packed into the tunneling wound, paste-form of the matrix or hydrogels are applied or packed directly into the wound, the latter preferred for its ease of use [Kim, Y. H., et al., A Prospective Randomized Controlled Multicenter Clinical Trial Comparing Paste-Type Acellular Dermal Matrix to Standard Care for the Treatment of Chronic Wounds. Journal of Clinical Medicine, 2022. 11 (8): p. 2203]. Examples of sheet-based wound dressings include Graftjacket® regenerative tissue matrix [Kirsner, R. S., et al., Human acellular dermal wound matrix: evidence and experience. International Wound Journal, 2015. 12 (6): p. 646-654] and AlloDerm [Yim, H., et al., The use of AlloDerm on major burn patients: AlloDerm prevents post-burn joint contracture. Burns, 2010. 36 (3): p. 322-328]. Sheet-type wound dressings best suit the superficial wounds but when it comes to tunneling wounds, they are ineffective [Kim, Y. H., et al., A Prospective Randomized Controlled Multicenter Clinical Trial Comparing Paste-Type Acellular Dermal Matrix to Standard Care for the Treatment of Chronic Wounds. Journal of Clinical Medicine, 2022. 11 (8): p. 2203]. The limitations include rolling of the sheet into required tunneling wound diameters, and incomplete packing of the wound which will result in stalled healing, secondary infections and other complications. Use of paste-type matrices or hydrogels overcome the limitations of the sheet-type dressings and has been proven to be effective for treating shallow tunneling wounds such as diabetic ulcers [Lee, M., et al., Clinical Efficacy of Acellular Dermal Matrix Paste in Treating Diabetic Foot Ulcers. Wounds: a Compendium of Clinical Research and Practice, 2019. 32 (1): p. 50-56.; Kim, S. W., et al., Application of paste-type acellular dermal matrix in hard-to-heal wounds. Journal of Wound Care, 2021. 30 (5): p. 414-418]. However, paste-type matrices are not ideal for deep tunnelling wounds. The disadvantages of paste-type matrix or hydrogels is felt during regular wound dressings as cleaning of the tunnels become extremely difficult and frequent dressings (to clean and pack the tunnels to prevent further infection) might expose the wound and the surrounding tissue to shear stresses and pressure, which might worsen the tunneling wounds. One solution is the use of bioprosthetic plugs such as Gore Bio-AR Fistula Plug [Nazari, H., et al., Advancing Standard Techniques for Treatment of Perianal Fistula; When Tissue Engineering Meets Seton: When tissue engineering meets seton for perianal fistula. Health Sciences Review, 2022: p. 100026] but there are limitations that include non-biodegradation or long-biodegradation time, dehydration of the wound site, and failure rates of ˜44% [Beaman, H. T., et al., Shape memory polymer hydrogels with cell-responsive degradation mechanisms for Crohn's fistula closure. Journal of Biomedical Materials Research Part A, 2022. 110 (7): p. 1329-1340]. A recent work reported use of shape memory polymer hydrogels based on polyvinyl alcohol (PVA) and cornstarch (CS) for treating Crohn's disease, a form of inflammatory bowel disease [Beaman, H. T., et al., Shape memory polymer hydrogels with cell-responsive degradation mechanisms for Crohn's fistula closure. Journal of Biomedical Materials Research Part A, 2022. 110 (7): p. 1329-1340]. However, the choice of materials is not ideal for tunneling wounds and lacks the ability to incorporate cells to accelerate the healing process in cases of tertiary wounds.

To overcome the above challenges associated with the treatment of deep tunneling wounds and limitations of the available solutions, a bioprinted tri-layered cellulose/collagen-based drug eluting fillers, referred to as tunneling wound fillers (TWF), are proposed as potential treatment option for deep tunneling wounds in this work. The role of collagen in wound healing is well-established through previous studies [Chen, K., et al., Pullulan-Collagen hydrogel wound dressing promotes dermal remodelling and wound healing compared to commercially available collagen dressings. Wound Repair and Regeneration, 2022. 30 (3): p. 397-408], especially marine collagen derived from fish [Liu, S., et al., Marine collagen scaffolds in tissue engineering. Current

Opinion in Biotechnology, 2022. 74: p. 92-103]. Cellulose and cellulose fibers were also explored previously as a potential wound dressing material [Chang, G., et al., Carboxymethyl chitosan and carboxymethyl cellulose based self-healing hydrogel for accelerating diabetic wound healing. Carbohydrate Polymers, 2022: p. 119687], with inclusion of anti-bacterial silver [Ohta, S., et al., Silver-loaded carboxymethyl cellulose nonwoven sheet with controlled counterions for infected wound healing. Carbohydrate Polymers, 2022. 286: p. 119289], or other growth factors [Diaz-Gomez, L., et al., 3D printed carboxymethyl cellulose scaffolds for autologous growth factors delivery in wound healing. Carbohydrate Polymers, 2022. 278: p. 118924]. Inclusion of cellulose fibers (micro/nano) also contributed to enhanced mechanical properties [Akatwijuka, O., et al., Overview of banana cellulosic fibers: agro-biomass potential, fiber extraction, properties, and sustainable applications. Biomass Conversion and Biorefinery, 2022: p. 1-17]. Hence, fish collagen and cellulose microfibers (CMFs) are chosen as the two main components of the TWF. Extrusion-based 3D Printing was used to fabricate TWF. Sodium alginate was used for suspending collagen-coated CMFs, mainly for post-printing crosslinking process that would impart structural stability to the printed TWF constructs.

The proposed TWF, which is bioprinted tri-layered cellulose/collagen-based drug eluting fillers, has many advantages compared to the current standard of care for tunneling wounds: (i) patient-specific and wound-specific customization based on the size of the wound, drug dosage depending on the healing rate, and possibility to incorporate patient-derived cells, (ii) made of CMFs/collagen-bioactive materials that mimics the natural extra-cellular matrix and provides a good balance between ease of insertion/removal while being structurally stable, (iii) smooth outer surface and soft, thereby reducing the pressure on the wound and the surrounding tissues preventing further tissue damage during insertion, removal and physiological movements, and (iv) good wound exudate absorption capability while releasing wound-healing drugs (the dosage of which can be controlled and modified in case of delayed healing). In addition to the above advantages, the source of the biomaterials used in this work are environmental-friendly and sustainable. Collagen is derived from fish skin that were discarded and CMFs were isolated from discarded banana stems (a kitchen waste), both contributing to the circular economy.

This work can be divided into four parts: (a) sustainable utilization and extraction of CMFs from banana stems; (b) encapsulating CMFs in fish skin-derived collagen, formulation and physicochemical characterization of bioactive hydrogel; (c) bioink formulation, drug encapsulation, 3D printing and bioprinting of TWFs and (d) application of biologically active, structurally stable, and functionally active TWFs for tunnel wound care application. After thorough characterization of the microstructural, physio-chemical, and mechanical properties, swelling rate, weight loss and drug release rate, the biocompatibility of 3D bioprinted TWFs was evaluated (Alamar Blue metabolic activity and LIVE/DEAD assay) with human mesenchymal stem cells (hMSCs) and in vitro wound healing evaluation using scratch test with Mouse embryonic fibroblasts (MEFs). Finally, the 3D-printed TWFs were tested for their suitability and applicability using the chicken tissue wound model for tunneling wound care strategy.

### Materials and Methods

### Isolation of CMFs

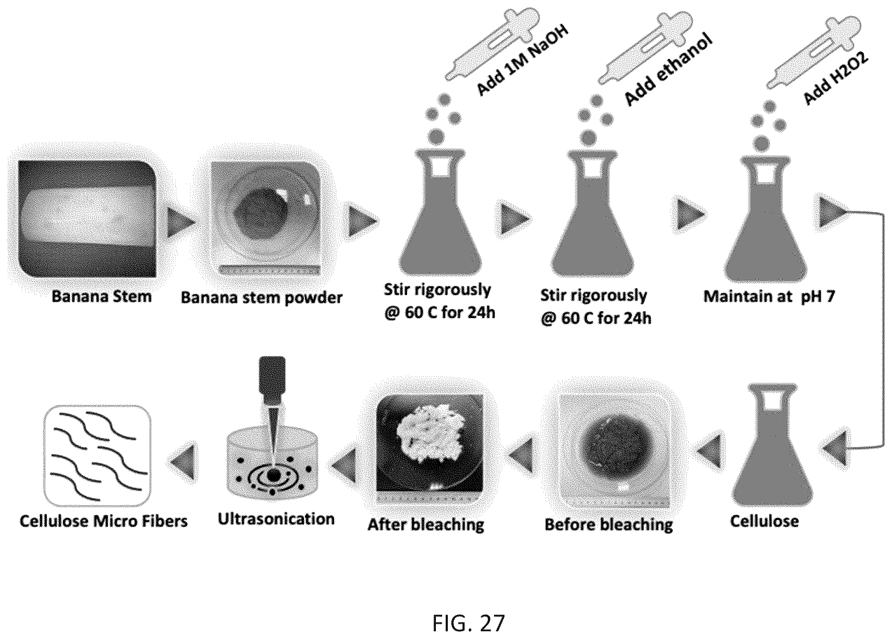

FIG. 27 shows the procedure to isolate CMFs from banana stems. Briefly, banana stems were collected from the kitchen waste and dried at 40° C. in hot air oven for three days, followed by air drying at room temperature and powdered using a kitchen mixer grinder. 100 mg of powdered fibers were soaked in 1M NaOH solution with vigorous stirring for 24 h at 60° C. The fibers were then washed with Millipore water until the pH reaches 7. The solution was filtered, washed, and lyophilized.

### Bleaching and Hydrolysis of Cellulose

The lyophilized powder was dissolved in 500 mL of H2O2 solution in a ratio of 1:1 at 75° C. for 30 minutes for bleaching [DN, J. and M. Jami, Extraction of microcrystalline cellulose (MCC) from cocoa pod husk via alkaline pretreatment combined with ultrasonication. International Journal of Applied Engineering Research, 2016. 11 (19): p. 9876-9879]. The sample was filtered and washed again with Millipore water. After bleaching, the banana stem derived cellulose fibers were treated with 1% (v/v) H2SO4 for 1 h at 80° C. After washing with water, the fibers were filtered and stored at 4° C. for the production of CMFs.

### Ultrasonication

Ultrasonication using an Ultrasonicater probe at specific amplitude of 70% for 4 hours was done to produce microfibers. To avoid overheating during sonication, the flask containing the fibers was placed in an ice bath. After sonication, the CMFs were separated using centrifugation at 4000 rpm for 30 minutes. The fibers were collected as pellets and stored at 4° C. for further studies.

Encapsulation of CMFs with Fish Skin-Derived Collagen

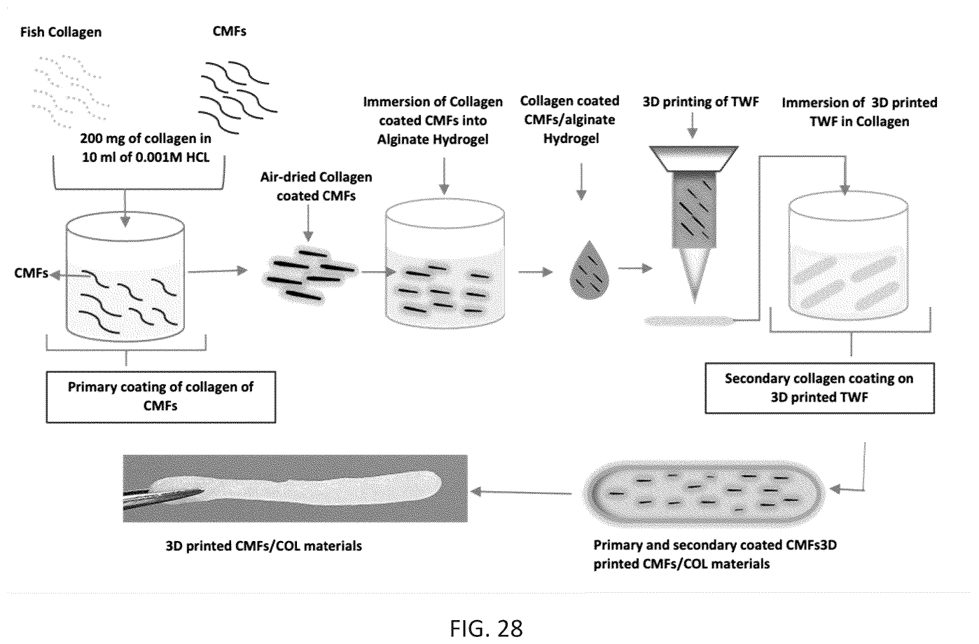

The discarded marine Grouper fish skin waste was sustainably utilized for the collagen extraction following a previously published procedure [Govindharaj, M., U. K. Roopavath, and S. N. Rath, Valorization of discarded Marine Eel fish skin for collagen extraction as a 3D printable blue biomaterial for tissue engineering. Journal of Cleaner Production, 2019. 230: p. 412-419]. 200 mg of collagen was diluted in 5 ml of phosphate buffered saline (PBS) with pH maintained at 7.4 with a magnetic stirrer and was then neutralized. The solution was incubated for 2 h at 37° C. for the hydrogel formation. CMFs in various ratios (25, 50, and 75 mg by weight) were prepared, named 25 CMFs, 50 CMFs, and 75 CMFS, respectively, based on the CMFs content. CMFs were mixed with the collagen solution and stirred overnight for complete encapsulation of CMFs. The hydrogels were then lyophilized and stored in 4° C. for hydrogel formulation. The procedure is illustrated graphically in FIG. 28.

### Characterization of CMFs-COL Hydrogel

The lyophilized CMFs/COL fibers were imaged using Quanta™ 450 FEG SEM. Agilent 670-FTIR spectroscopy was used for the analysis of the functional group of CMFs/COL hydrogel in the range of 400-4000 cm-1. Thermogravimetric analyzer SDT Q600 Instrument was used to evaluate the thermal stability of CMFs-COL fibers. To check the crystalline structure of CMFs materials, X-ray diffraction (XRD) using Malvern Panalytical Empyrean 3 was used at room temperature from 2-400 (20). To confirm the successful collagen coating on CMF fibers, the Raman spectroscopy (WITec alpha 300 equipment) was used and spectra was recorded from 0-3500 using a 600-mW laser. The mechanical and compressive properties of 3D printed TWF were measured using the MACH-1 v500 (Biomomentum Inc. Canada) instrument.

### 3D Printing of Tunneling Wound Fillers (TWFs)

### Rheology Tests

Sodium alginate was used for suspending collagen-coated CMFs, mainly for post-printing crosslinking process that would impart structural stability to the printed TWF constructs. 25 mg of sodium alginate (SA) (Spectrum® Chemical MGF.CORP., Gardena, CA) is added to the three CMFs/COL lyophilized powder in 4 ml of DMEM media and was stirred thoroughly to develop a 3D printable ink. ElastoSens Bio 2 Rheometer was used to characterize the rheological properties of different concentrations of CMFs hydrogel. A minimum volume of 4 ml hydrogel was required for each test. One test lasted 90 minutes to reach a steady state. All the tests were performed at 24° C. (room temperature). Shear storage modulus and shear loss modulus over time were obtained.

### 3D Printing of TWF Hydrogel

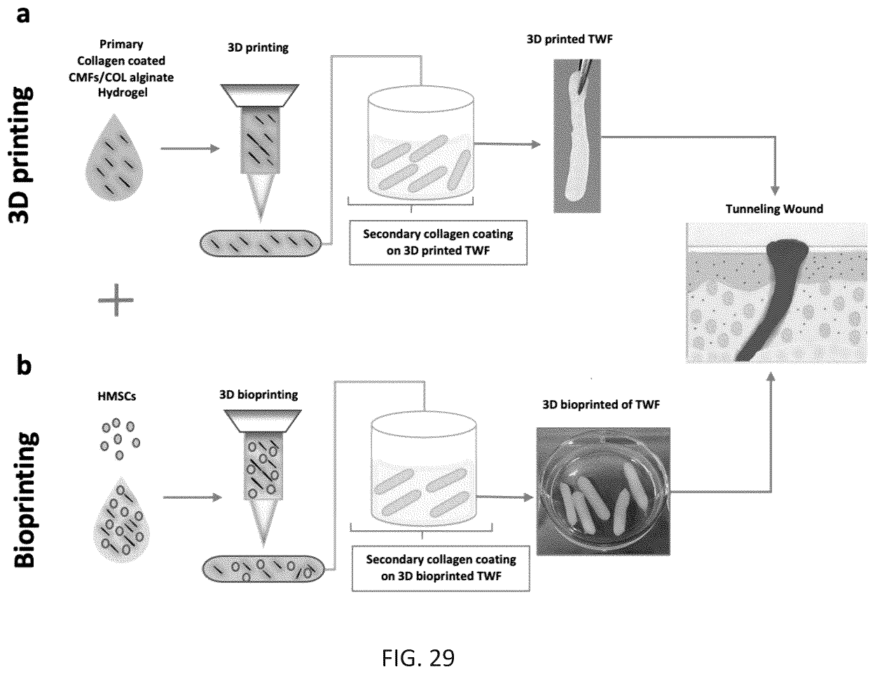

RegenHU 3D-Discovery Bioprinter (RegenHU Ltd, Switzerland) was used to 3D print TWFs (FIG. 29A). 4 ml of formulated ink was filled into the cartridge and printed using a conical needle of internal diameter 1.5 mm, feed rate 4 mm/s and a pressure of 0.03 MPa. After successful printing, the construct was ionically crosslinked with 250 mM CaCl2 for 5 minutes to get a stable construct and washed gently with PBS solution. These constructs were then used for subsequent characterizations and experiments.

### Swelling Degradation Test

To analyze the swelling ratio (SR), lyophilized TWFs were weighed (Wi) and soaked in phosphate-buffered saline (PBS) solution at room temperature. At specified time points (1, 6, 12, 24, 48, and 72 hours), the sample was taken out and weighed (Wt). SR is given by the equation,

$$SR = ( Wt - Wi ) / Wi * 100 \% \qquad Equation 1$$

Similarly, the degradation rate (DR) experiments were conducted with specific modifications. The TWF sample was soaked in PBS and taken out at the defined time points, lyophilized, and then weighed (Wdt) WL is given by the equation,

$$DR = ( Wi - Wdt ) / Wi * 100 \% \qquad Equation 2$$

### In Vitro Release Rate of Collagen and Baneocin

An in vitro drug release study was performed to analyze the drug release rate of TWFs coated with collagen and Baneocin powder (a common skin antibiotic drug). Collagen and Baneocin coated TWFs were immersed in 10 mL of simulated body fluid (pH 7.4 PBS) at 37° C. and gently rotated at 100 rpm. At predetermined intervals (0, 6, 12, 24, 48, and 72 hours), 1 mL PBS solution was taken out while the same volume of fresh PBS was replaced to maintain the original volume of 10 ml. The amount of collagen and Baneocin released was determined by measuring the absorbance at 520 nm. All experiments were performed in triplicates.

### Biocompatibility Evaluation of 3D Bioprinted TWF

### Cell Culture

Human mesenchymal stem cells (hMSCs) were cultured in DMEM media (DMEM supplemented with 2 mM L-glutamine, 5% fetal bovine serum (FBS), and 1% penicillin-streptomycin). The culture was further maintained in an incubator (5% CO₂, 37° C.) for proliferation. Cells were trypsinized, sub-cultured and harvested for 3D bioprinting after confluency. All the bioink preparation was carried out under sterile cell culture conditions in a laminar airflow chamber. The 3D bioprinting hood was properly sterilized using 70% ethanol and UV sterilized 4 hours before starting the printing. Each 3D CMFs/COL based TWF constructs were bioprinted with 200,000 cells for all the subsequent biological experiments.

### Bioink Preparation and Bioprinting

The bioink preparation was carried out using the procedure described in a previous article [Govindharaj, M., et al., 3D Bioprinting of human Mesenchymal Stem Cells in a novel tunic decellularized ECM bioink for Cartilage Tissue Engineering. Materialia, 2022: p. 101457]. FIG. 29B shows the schematic representation of the 3D bioprinting process. In detail, 50 CMFs/COL bioink lyophilized materials were mixed with the 25 mg of sodium alginate (SA) (Spectrum® Chemical MGF.CORP., Gardena, CA) in 4 ml of DMEM media to develop a 3D printable ink; hMSCs were then mixed (200,000 cells/construct) and loaded into a 3 ml extrusion cartridge, and bioprinting was done with a needle of 1.5 mm internal diameter, at a feedrate of 4 mm/s and a pressure of 0.3 MPa. After successful bioprinting, the TWF constructs were ionically crosslinked with 250 mM CaCl₂) for 5 minutes, followed by 15 minutes of incubation with 0.2% FBS solution. Then the bioprinted construct was immediately transferred to cell culture media (DMEM with 10% FBS and 1% penicillin-streptomycin) and incubated at standard culture conditions.

### Cell Viability and Proliferation

To quantitatively analyze the cell proliferation of hMSCs in 3D bioprinted TWF constructs, the Alamar Blue (AB) assay (BioSource International, Camarillo, CA, USA) was used to measure the metabolic activity as per the manufacturer's protocol. LIVE/DEAD staining with Calcein AM and Ethidium homodimer1 (LIVE/DEAD™ Viability/Cytotoxicity Kit, Thermo Fisher Scientific, USA) was performed to visualize the viability of hMSCs in the 3D constructs as per the manufacturer's protocol. Imaging was done using Leica SP8 confocal laser scanning microscope.

### In Vitro Wound Healing Assay

In vitro cell migration studies with Mouse embryo fibroblasts (MEFs) cells was performed to evaluate the wound healing potential using a previously described method [Bolla, S. R., et al., In vitro wound healing potency of methanolic leaf extract of Aristolochia saccata is possibly mediated by its stimulatory effect on collagen-1 expression. Heliyon, 2019. 5 (5): p. e01648]. Briefly, 200,000 cells/mL were seeded in 6-well plates and were cultured overnight. Cells were then washed with PBS and a scratch was gently created using a sterile pipette tip (200 μL diameter). PBS solution were used to remove the detached cells by gentle washing. Cells were treated with 100 μL of collagen solution and Baneocin extract and incubated for 24 h. Baneocin is a standard drug that is used in wound healing. Untreated cells were negative control. Images were taken using an inverted microscope. All experiments were performed in triplicates (n=3).

### Chicken Tissue Model for Tunneling Wounds

To check the suitability and applicability of 3D printed TWFs, a chicken breast tissue wound model was developed. Briefly, whole chicken tissue was purchased from the market and two full-thickness wounds were created on the side of the breast with deep tunneling formation (1.5 cm depth) using a surgical knife. Then, 3D printed TWFs was inserted into the deep tunneling wounds to check the applicability of TWFs.

### Results and Discussion

### Extraction, Formulation, and Characterization of CMF COL Inks

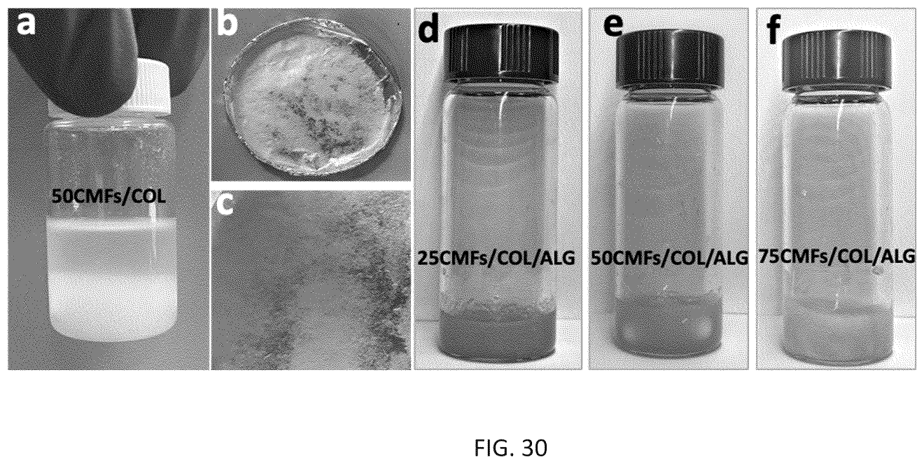

After the step-by-step isolation process as described in the methods section, CMFs were coated with fish skin-derived collagen (COL) (FIG. 30A through FIG. 30C) to improve the bioactivity. Representative images of 50 CMFs/COL are shown in FIG. 30A through FIG. 30C, the same procedure is followed for the other two concentrations. This is the first and primary collagen coating.

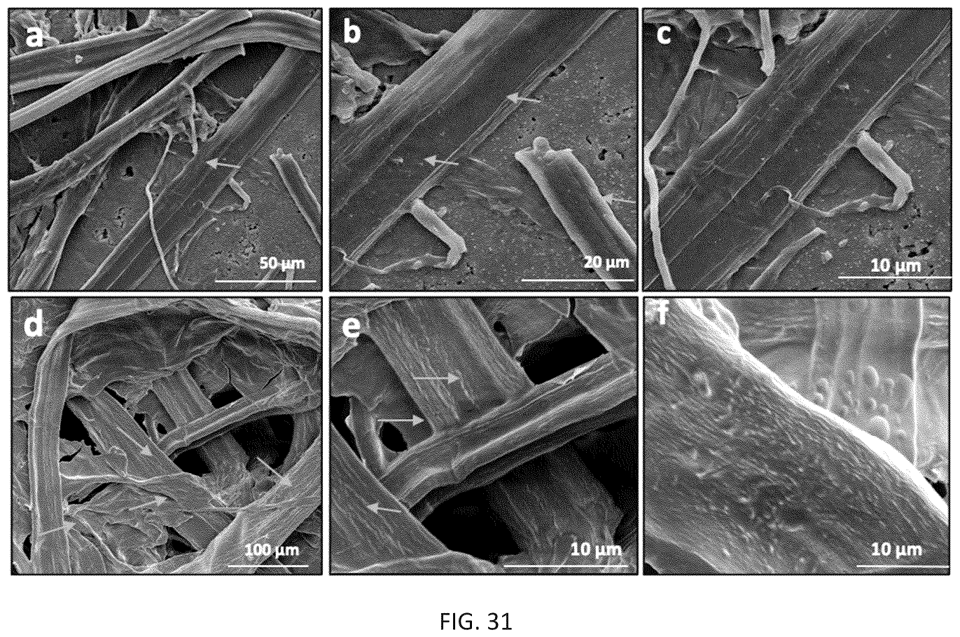

The difference between the surfaces coated with collagen and those without collagen can be clearly seen in the SEM images shown in FIG. 31. CMFs without primary collagen coating has a smooth surface (FIG. 31A through FIG. 31C) while the surface of CMFs coated with collagen has a rough and wavy surface (FIG. 31D through FIG. 31F). It can also be seen from FIG. 31D through FIG. 31F that the CMFs are completely covered by the collagen, proving the effectiveness of the coating.

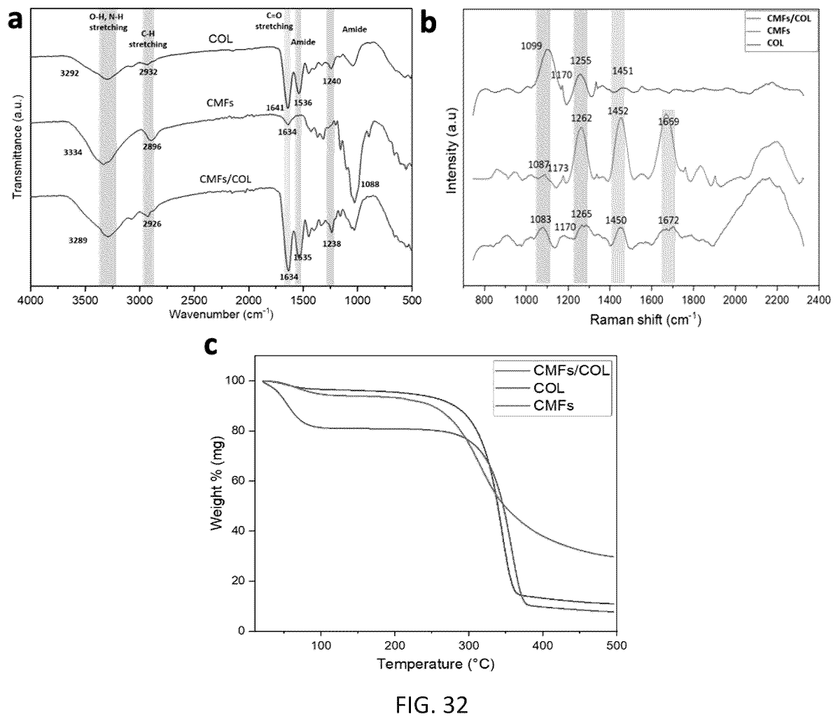

To support the observations made by SEM analysis, CMFs, collagen, and CMFs/COL ink were characterized by FTIR to determine the presence of collagen over CMFs. The FTIR spectra of CMFs, COL (collagen), and 50 CMFs/COL ink are shown in FIG. 32A. The peaks seen in 50 CMFs/COL ink around 3289, 2926, 1634, and 1535 cm-1 correspond to the O—H, N—H, C—H, C═O, and amide stretching vibration. These are the main functional groups for CMFs and collagen constituents, which were also present in pure CMFs and pure collagen. In the CMFs/COL ink, the bands of N—H and O—H stretching were shifted to the lower wavenumber of 3289 cm-1 (that of CMFs at 3334 cm-1) and the C═O bands of CMFs/COL shifted to 1665 cm-1. Some shifts in the characteristic peaks of both CMFs and collagen were observed in the CMFs/COL ink.

The broad peaks at 3289 cm-1 which is the indication of N—H stretching vibration of amine groups of fish-derived collagen could interact with the O—H stretching of CMFs [Lohrasbi, S., et al., Collagen/cellulose nanofiber hydrogel scaffold: physical, mechanical and cell biocompatibility properties. Cellulose, 2020. 27 (2): p. 927-940; Liu, C.-Y., et al., Collagen/cellulose nanofiber blend scaffolds prepared at various pH conditions. ACS Applied Bio Materials, 2018. 1 (5): p. 1362-1368].

Further, the successful coating of CMFs fibers with collagen was confirmed by Raman spectroscopic analysis (FIG. 32B), indicating the presence of cellulose and collagen in the CMFs/COL formulation. The spectral data of lyophilized CMFs, collagen and CMFs/COL material showed clear peaks at 1099 cm-1, 1170 cm-1,1255 cm-1, and 1452 cm-1, indicating the presence of collagen [Połomska, M., et al., Effects of Temperature on the FT NIR Raman Spectra of Fish Skin Collagen. Applied Sciences, 2021. 11 (18): p. 8358]. The characteristic peaks representing cellulose at 1083, 1262, and 1452 cm-1 were also observed [Govindharaj, M., et al., Bioprinting of bioactive tissue scaffolds from ecologically-destructive fouling tunicates. Journal of Cleaner Production, 2022. 330: p. 129923]. The main characteristic peaks for both CMFs, and collagen were present in the CMFs/COL ink. These results clearly confirm the successful coating of collagen on the CMFs.

FIG. 32C shows the TGA thermograms of CMFs, COL, and CMFs/COL ink. For collagen, major weight loss occurred in the range of 200° C.-500° C. and for CMFs it occurred between 300-400° C. (indicating the decomposition of cellulose). In both collagen and CMFs, the first thermal degradation mainly indicates the loss of free and bound water molecules [Govindharaj, M., et al., Bioprinting of bioactive tissue scaffolds from ecologically-destructive fouling tunicates. Journal of Cleaner Production, 2022. 330: p. 129923]. Data shows that the thermal stability of CMFs/COL composite was improved compared to pure collagen due to the crosslinking and association of cellulose and collagen molecules [Yang, Q., et al., Improved thermal and mechanical properties of bacterial cellulose with the introduction of collagen. Cellulose, 2017. 24 (9): p. 3777-3787]. While the onset temperature of pure collagen was around 150 to 250° C., the thermal degradation range of both CMFs and CMFs/COL was around 300-400° C., with more than 70% weight loss happening within this range.

### Rheology, 3D Printing, and Secondary Collagen Coating of CMF's COL ALG Inks

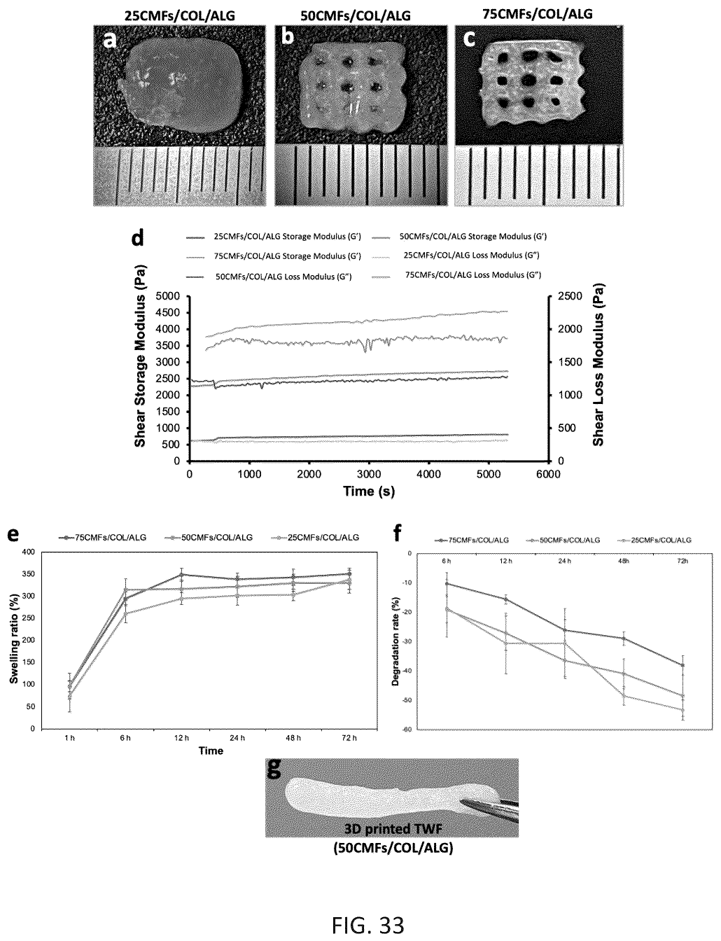

Three different concentrations of CMFs/COL/ALG inks (25, 50, and 75 CMFs/COL/ALG) were prepared as shown in FIG. 30D through FIG. 30F. As indicated earlier, alginate was added to the CMFs/COL hydrogels to enable post-printing ionic crosslinking for structural stability. The printability of the three ink formulations (25, 50, and 75 CMFs/COL/ALG) were evaluated as shown in FIG. 33A through FIG. 33C. While the 25 CMFs/COL/ALG ink completely covered the pores, 50 and 75 CMFs/COL/ALG inks had better printability. Viscosity of the inks increased with increasing concentration of CMFs, with 25 CMFs/COL/ALG with the lowest viscosity and 75 CMFs/COL/ALG with the highest viscosity. The lower viscosity of the 25 CMFs/COL/ALG ink explains why the printed structure is completely devoid of pores. With increasing CMFs concentration and increasing viscosity, the printability improves. This can also be explained by the rheological properties of these inks shown in FIG. 33D. While all the three inks had a higher storage modulus than loss modulus depicting that the ink behaves more as a gel than a solution, the magnitude of the modulus increases with increasing CMFs concentration as seen in FIG. 33D. Viscosity and modulus not only affect the printability but also post-printing cell viability due to high shear stress [Chand, R., B. S. Muhire, and S. Vijayavenkataraman, Computational Fluid Dynamics Assessment of the Effect of Bioprinting Parameters in Extrusion Bioprinting. International Journal of Bioprinting, 2022. 8 (2)]. Hence, the 75 CMFs/COL/ALG ink was not a choice due to the very high storage modulus while the 25 CMFs/COL/ALG ink failed the printability test. 50 CMFs/COL/ALG ink is chosen for further experiments. Further optimizations of these inks and printing parameters are possible but since the focus was to address the challenges in treating tunneling wounds, 50 CMFs/COL/ALG ink is selected. FIG. 33G shows the 3D printed TWF using 50 CMFs/COL/ALG ink.

FIG. 33E shows the swelling ratio of the three different CMFs/COL/ALG based TWF constructs. Swelling ratio increased exponentially until 6 hours (around 250-300% from the initial dry weight) after which the fluid uptake stabilized to an equilibrium state. Presence of hydrophilic groups in the CMFs contributes to these high values of swelling ratio [Baniasadi, H., et al., Direct ink writing of aloe vera/cellulose nanofibrils bio-hydrogels. Carbohydrate Polymers, 2021. 266: p. 118114]. This is proven by the data (FIG. 33E) where increased concentration of CMFs had a higher swelling ratio. For example, after 12 hours, the swelling ratios of 25, 50 and 75 CMFs/COL/ALG inks were 294±14, 316±21, and 348±15% respectively. Good swelling properties provide an appropriate 3D structure and favorable environment for cell infiltration, nutrient diffusion, cell migration, and removal of waste products [Govindharaj, M., et al., 3D Bioprinting of human Mesenchymal Stem Cells in a novel tunic decellularized ECM bioink for Cartilage Tissue Engineering. Materialia, 2022: p. 101457; Baniasadi, H., et al., Direct ink writing of aloe vera/cellulose nanofibrils bio-hydrogels. Carbohydrate Polymers, 2021. 266: p. 118114]. Moreover, the relatively higher fluid uptake capability of these hydrogels might be an advantage for wound dressing and tissue engineering applications [Nazarnezhada, S., et al., Alginate hydrogel containing hydrogen sulfide as the functional wound dressing material: In vitro and in vivo study. International Journal of Biological Macromolecules, 2020. 164: p. 3323-3331].

FIG. 33F shows the weight-loss profile of TWF constructs monitored for 72 h. A controlled weight loss over time was observed in all three groups. Degradation in weight is contributed by two factors, the release of collagen and breaking of the crosslinked polymeric chains. Even as the weight decreased, the structural stability of the constructs was preserved to some extent that it did not completely disintegrate and maintained its cord-like tubular form (shown in FIG. 33G). It can also be seen in FIG. 33F that the rate of degradation mostly follows the CMFs concentration, the more the CMF concentration, lesser the degradation rate. TWF constructs with higher content of CMFs were subjected to lower weight loss due to higher crystalline structured cellulose materials undergoing degradation in specific enzymatic, autocatalytic, and hydrolytic conditions [Baniasadi, H., et al., Direct ink writing of aloe vera/cellulose nanofibrils bio-hydrogels. Carbohydrate Polymers, 2021. 266: p. 118114]. By tuning the concentrations of CMFs, the degradation and in turn the drug release rate (when loaded with drugs) can be controlled, opening the possibility of controlled drug release [Milojević, M., et al., Hybrid 3D printing of advanced hydrogel-based wound dressings with tailorable properties. Pharmaceutics, 2021. 13 (4): p. 564].

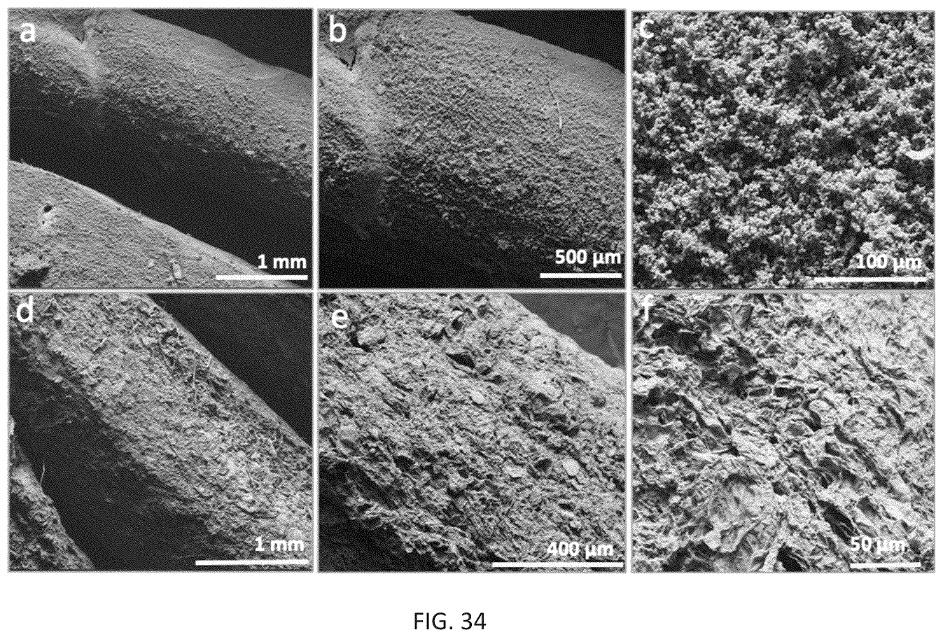

Once the constructs are printed, the secondary collagen coating was done over the printed TWF constructs (tubular constructs shown in FIG. 33G). SEM images of the printed constructs with and without secondary collagen are shown in FIG. 34. Similar to the observations after primary collagen coating, the surfaces without secondary collagen coating (FIG. 34A through FIG. 34C) were smooth compared to the surfaces with secondary collagen coating (FIG. 34D through FIG. 34F).

### Drug-Loaded TWF's

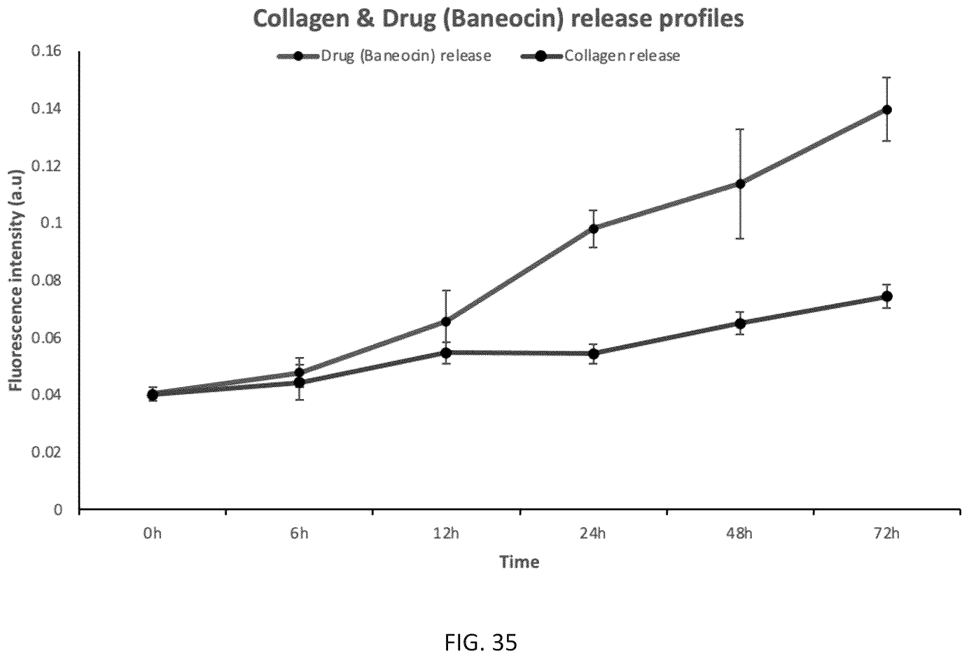

Baneocin is a common drug used for treating skin wounds. Prospects of drug-loaded TWFs for wound healing was evaluated with Baneocin. Secondary collagen coating was compared with Baneocin coating and the release profiles of Collagen and Baneocin is shown in FIG. 35. To remove any ambiguity, two sets of TWFs were used in this study. The first set of TWFs are the same as fabricated before, shown in FIG. 33G, with a secondary collagen coating on 3D printed TWFs (50 CMFs/COL/ALG). The second set consisted of 3D printed TWFs (50 CMFs/COL/ALG) coated with Baneocin solution instead of secondary collagen coating, with all the other procedures and parameters being the same. Both the profiles show an increasing release with time as expected. Generally, the release stages can be divided into three phases namely burst release phase, non-linear monotonic release phase, and final release phase [Huo, P., et al., Electrospun nanofibers of polycaprolactone/collagen as a sustained-release drug delivery system for artemisinin. Pharmaceutics, 2021. 13 (8): p. 1228]. However, the burst release phase is not seen in the curve rather only a gradual release with time. This might be due to the limited time period of evaluation (72 hours) and a longer time duration might reveal all three phases of drug release. Comparing the collagen and Baneocin release profiles, Baneocin exhibited a faster release rate as it dissolves much faster than collagen. Collagen-loaded TWFs showed slow release rate due to their gelling properties and viscous nature [Kawamata, H., et al., Hierarchical viscosity of aqueous solution of tilapia scale collagen investigated via dielectric spectroscopy between 500 MHz and 2.5 THz. Scientific reports, 2017. 7 (1): p. 1-8] suitable for deep wound healing and tissue regeneration. These results suggest that 3D printed TWFs are suitable for sustained drug delivery applications in tunneling wound care treatments.

### Fabrication, 3D Printing and Bioprinting of TWF

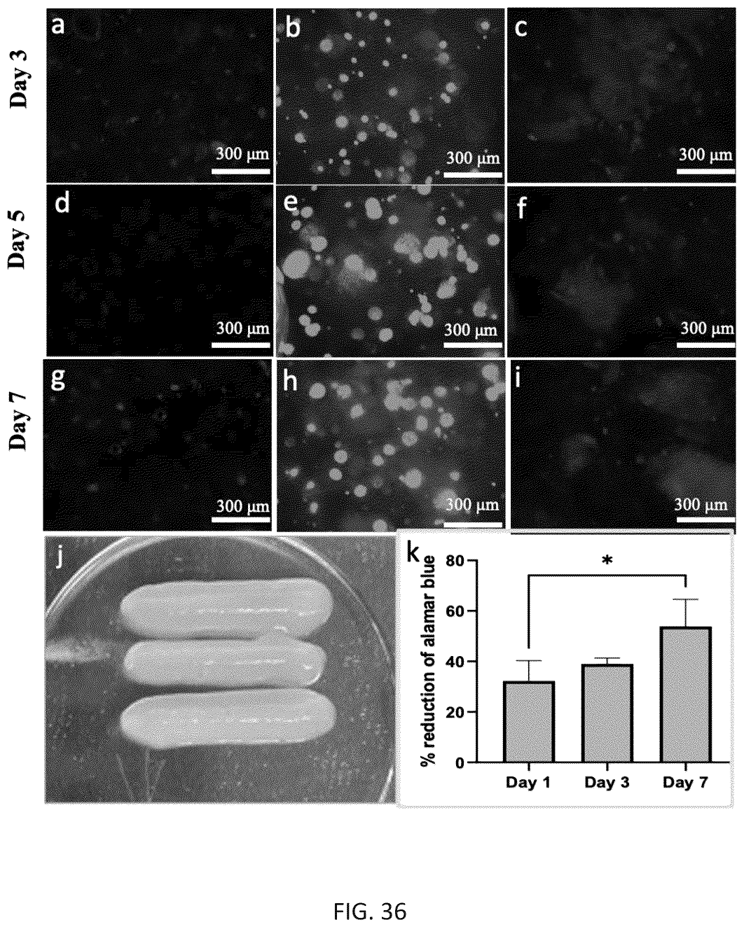

The use of stem cells for treating various diseases is an emerging and promising trend in healthcare. After successfully proving that drug-loaded TWFs are possible to fabricate, the example moved to incorporate stem cells into the 50 CMFs/COL/ALG ink to bioprint cell-laden TWFs. Human mesenchymal stem cells (hMSCs) were used in this study as hMSCs are multipotent with the ability to differentiate into several lineages including bone, cartilage, fat, and skin. FIG. 36J shows the bioprinted hMSCs-laden TWFs. The bioink formulated by mixing hMSCs in the 50 CMFs/COL/ALG ink supported the survival, growth, and proliferation of cells as shown in FIG. 36. Live/dead staining and DAPI staining images of hMSCs on day 3 are shown in FIG. 36B and FIG. 36C respectively, while FIG. 36A shows a no cell control for day 3 (with the faint green staining due to autofluorescence of the ink itself). Similarly, the Live/dead staining and DAPI staining images of hMSCs on day 5 are shown in FIG. 36E and FIG. 36F respectively (FIG. 36D is a no cell control for day 5) and for day 7 are shown in FIG. 36H and FIG. 36I respectively (FIG. 36G is a no cell control for day 7). Cell viability was also quantitatively measured using the AlamarBlue assay, the results of which shown in FIG. 36K corroborate the qualitative live/dead and DAPI staining results. There is a significant cell proliferation on day 7 compared to day 1, as seen in FIG. 36K. The bioink developed in this work, which is a cellulose-collagen blend, provides a favorable 3D microenvironment for cell adhesion, migration and proliferation. These results indicate that cell-laden TWFs can be bioprinted with a potential to treat tertiary tunneling wounds where stem cells could be used to augment the healing process or in regeneration of the damaged tissues.

### Scratch Test to Evaluate In Vitro Wound Healing

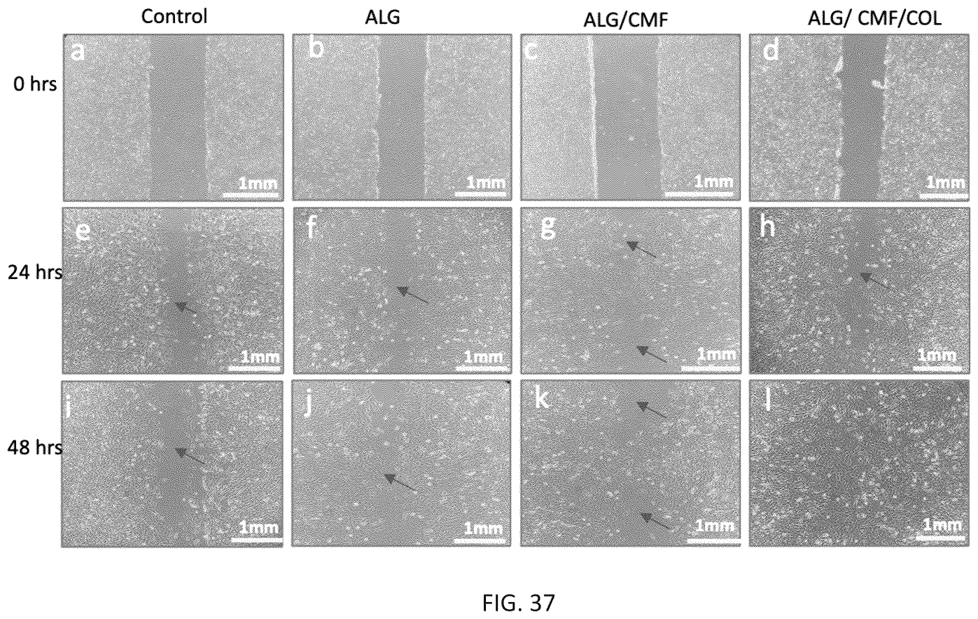

The migratory and proliferative abilities of the fibroblasts play a pivotal role in wound healing [Bolla, S., Mohammed Al-Subaie A, Yousuf Al-Jindan R, Papayya Balakrishna J, Kanchi Ravi P, Veeraraghavan V P, Arumugam Pillai A, Gollapalli S S R, Palpath Joseph J, Surapaneni K M. vitro wound healing potency of methanolic leaf extract of Aristolochia saccata is possibly mediated by its stimulatory effect on collagen-1 expression. Heliyon, 2019. 5: p. e01648]. In order to evaluate the wound healing potential, 100 μL of the extract of pure alginate (ALG), CMFs/ALG, and CMFs/COL/ALG were added to each well containing mouse embryonic fibroblasts (MEFs). Results are shown in FIG. 37, with FIG. 37A through FIG. 37D showing the image taken immediately after scraping the cell layer forming a breach and the migratory potential of MEFs after 24 and 48 hours shown in FIG. 37E through FIG. 37H (24 hours) and FIG. 371 through FIG. 37L (48 hours) respectively. While MEFs migrated well with all the three hydrogels, the ones treated with CMFs/COL/ALG was the best and almost completely covered the scratched wound areas in 48 hrs. The other two groups ALG and CMFs/ALG, while definitely better than the control, did not completely cover the area possibly due to the lack of collagen as collagen plays a critical role in tissue formation during wound regeneration [Ehrlich, H. P. and T. K. Hunt, Collagen organization critical role in wound contraction. Advances in wound care, 2012. 1 (1): p. 3-9]. These results strongly indicate that the collagen incorporated CMFs/COL/ALG ink might accelerate the wound healing process. Specifically, fish-derived collagen were known to enhance the fibroblast and keratinocytes migration within the wound area by activating the genes responsible for wound healing [Elbialy, Z. I., et al., Collagen extract obtained from Nile tilapia (Oreochromis niloticus L.) skin accelerates wound healing in rat model via up regulating VEGF, bFGF, and α-SMA genes expression. BMC veterinary research, 2020. 16 (1): p. 1-11] and due to its similarities with human dermal collagen [Furtado, M., et al., Development of fish collagen in tissue regeneration and drug delivery. Engineered Regeneration, 2022].

### Suitability of 3D) Printed TWF on Chicken Tissue Wound Model

A chicken tissue model was used to demonstrate the suitability of using 3D printed TWFs for tunneling wound applications. Two deep tunnel wounds of diameter 5 mm and length 1.5 cm were made on the chicken tissue as described in the methods section and the 3D printed TWFs were inserted into the wounds.

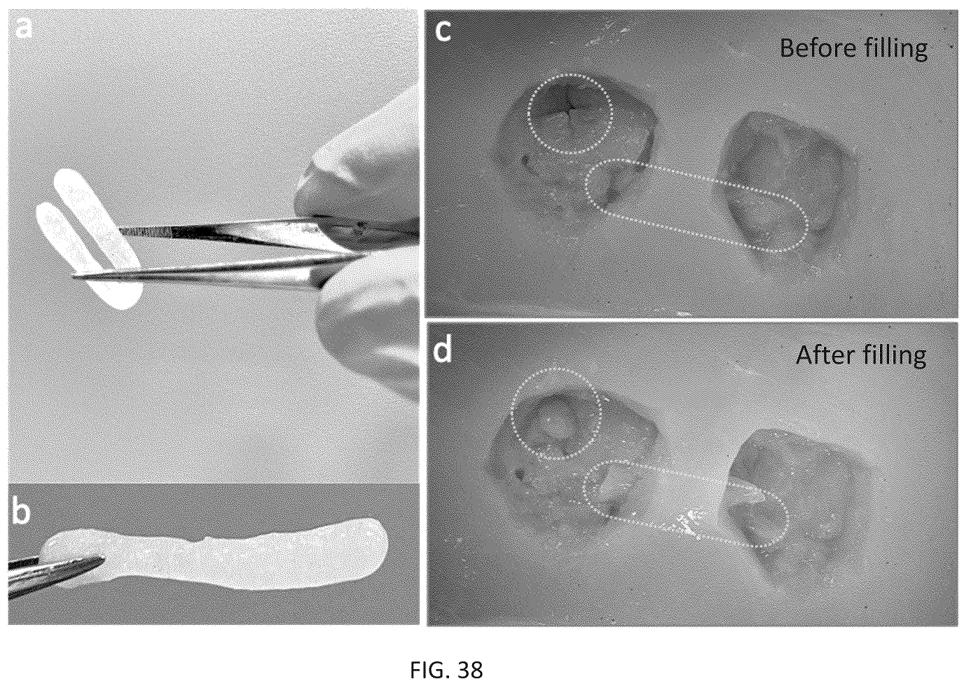

FIG. 38A and FIG. 38B shows the 3D printed TWFs that were inserted into the wounds. As shown in FIG. 38A, the structural stability and flexibility of 3D printed 50 CMFs/COL/ALG TWF indicated that the TWF hydrogel is suitable for inserting into the deep tunneling wounds with twists and turns. The 3D printed TWF revealed a smooth surface and softly curved edges with a moist environment (FIG. 38B). After creating two deep tunneling wounds (FIG. 38C), each wound was filled with 3D printed TWFs (FIG. 38D). The 3D printed CMFs/COL/ALG-based TWF was smoothly inserted, penetrated into the tunnel wound and the wound is completely packed. The softness and the moist TWFs could make a smooth contact with the surrounding damaged tissue in deep tunnel wounds and also provide a favorable condition for healing [Li, J., et al., Moist-retaining, self-recoverable, bioadhesive, and transparent in situ forming hydrogels to accelerate wound healing. ACS Applied Materials & Interfaces, 2020. 12 (2): p. 2023-2038].

### Conclusion

A proof-of-concept work on fabricating 3D printed tri-layered drug-eluting tunnel wound fillers for treating deep tunneling wounds was successfully demonstrated in this work. Extraction of cellulose microfibers from banana stem and coating them with fish skin-derived collagen yielded a good blend that mimics the dermal extracellular matrix. Three different concentrations of CMFs (25, 50, and 75 mg) were tested. While the lowest CMF concentration had less viscosity and poor printability, the highest concentration was very viscous and will affect the suspended cells during bioprinting. Hence, the 50 CMFs/COL/ALG was chosen and TWFs were fabricated. The structural stability and flexibility of 3D printed 50 CMFs/COL/ALG TWFs indicated that they are suitable for inserting into the deep tunneling wounds with twists and turns, as confirmed by the chicken wound model. Drug-eluting TWFs incorporating Baneocin showed controlled drug release rate and opens up the possibility for fabricating TWFs with different drugs for wound healing and tunable release rate depending on the healing progression. Bioprinting of hMSCs-laden TWFs showed that the optimized bioink supports cell survival, growth, and proliferation. The proposed TWF, which is bioprinted tri-layered cellulose/collagen-based drug eluting fillers, has many advantages compared to the current standard of care for tunneling wounds: (i) patient-specific and wound-specific customization based on the size of the wound, drug dosage depending on the healing rate, and possibility to incorporate patient-derived cells, (ii) made of CMFs/collagen bioactive materials that mimics the natural extra-cellular matrix and provides a good balance between ease of insertion/removal while being structurally stable, (iii) smooth outer surface and soft, thereby reducing the pressure on the wound and the surrounding tissues preventing further tissue damage during insertion, removal and physiological movements, and (iv) good wound exudate absorption capability while releasing wound-healing drugs (the dosage of which can be controlled and modified in case of delayed healing). In addition to the above advantages, the source of the biomaterials used in this work are environmental-friendly and sustainable. Collagen is derived from fish skin that were discarded and CMFs were isolated from discarded banana stems (a kitchen waste), both contributing to the circular economy. While this is a proof-of-concept work, future studies are required to further optimize the bioink composition, possible differentiation of stem cells post-printing, and in vivo animal studies before possible clinical translation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A biomaterial comprising at least one component derived from at least one sustainable source.

2. The biomaterial of claim 1, wherein the at least one sustainable source is selected from the group consisting of: tunicate, fish skin, algae, banana skin or stem, and watermelon, grouper, Polyclinum constellatum, and Pallusia nigra.

3. The biomaterial of claim 2, wherein the at least one component is selected from the group consisting of: extracellular matrix (ECM), ECM proteins, decellularized extracellular matrix (dECM), lyophilized dECM, collagen, cellulose, cellulose microfibers (CMFs) and alginate.

4. The biomaterial of claim 3, wherein the biomaterial further comprises at least one additive.

5. The biomaterial of claim 4, wherein the at least one additive is selected from the group consisting of: biopolymers, synthetic polymers, cross-linking agents, surfactants, and drugs/therapeutics.

6. The biomaterial of claim 5, wherein the biomaterial is selected from the group consisting of: bioink, hydrogels, microparticles, wound dressing, tissue engineering constructs, scaffolds, substrates and tunneling wound fillers (TWFs).

7. The biomaterial of claim 6, wherein the biomaterial further comprises one or more cells.

8. The biomaterial of claim 7, wherein the one or more cells is selected from the group consisting of: fibroblasts, neural stem cells, er-mesenchymal stem cells, and cells derived from induced pluripotent stem cell.

9. (canceled)

10. (canceled)

11. (canceled)

12. (canceled)

13. (canceled)

14. The biomaterial of claim 8, wherein the biomaterial further comprises at least one component selected from the group consisting of: a hydrogel, alginate, Matrigel, and a bioink.

15. (canceled)

16. (canceled)

17. (canceled)

18. The biomaterial of claim 1, wherein the biomaterial comprises (a) collagen from fish skin, and (b) a CMFs from banana.

19. The biomaterial of claim 18, wherein the collagen is derived from grouper, and/or the CMFs are derived from banana stem.

20. (canceled)

21. The biomaterial of claim 18, wherein the biomaterial comprises at least one component selected from the group consisting of: alginate, Matrigel, Baneocin and a bioink.

22. (canceled)

23. (canceled)

24. The biomaterial of claim 21, wherein the biomaterial is a tunneling wound filler (TWF).

25. The biomaterial of claim 24, wherein the TWF comprises a tri-layer coating comprising a first layer comprising CMF, a second layer comprising collagen, and a third layer comprising collagen.

26. The biomaterial of claim 18, wherein the biomaterial comprises mesenchymal stem cells.

27. (canceled)

28. A method comprising:
generating tunicate-derived decellularized extracellular matrix (dECM) by decellularizing tunicate tissue;
lyophilizing the decellularized tissue; and
forming a scaffold from the tunicate-derived dECM.

29. The method of claim 28, wherein the method further comprises powderizing the dECM and forming a bioink from the powderized dECM.

30. The method of claim 29, wherein the method comprises 3D printing the bioink.

31. A kit comprising a 3D printer and (a) a bioink comprising tunicate-derived decellularized extracellular matrix (dECM), or (b) a bioink comprising fish-derived collagen and banana-derived CMFs, or (c) a bioink for printing TWFs comprising fish-derived collagen and banana-derived CMFs.

32. A method for generating a sustainable biomaterial, comprising the steps of:
isolating CMFs from banana;
harvesting collagen from fish skin;
coating the banana-derived CMFs with the fish-derived collagen;
coating the collagen coated CMFs with an additional layer of fish-derived collagen; and
combining with Sodium Alginate.

33. The method of claim 32, wherein the method further comprises powderizing the biomaterial and forming a bioink from the powderized biomaterial.

34. The method of claim 33, wherein the method comprises 3D printing the bioink.

35. (canceled)

36. (canceled)
